SEQUENCE LISTING (108 sequences)

SEQ ID NO: 1 - DNA, 1584 bp, Oryza sativa
atggcgggagggcagctcaacgtgctgagcacgctcgaccaggcgaagacgcaatggtaccacttcatggcgatcgtcatcgccggcatgggcttcttcaccgacgcctacgacctcttctgcatttccctcgtcaccaagctgctcggccgcatctactacaccgacgattccaaggacacccccggcgcgctcccgcccaacgtgtcggccgccgtcaccggcgtcgcgctctgcggcacgctcgccggccagcttttcttcggatggctcggcgacaagctcggacgcaagagcgtgtatggtttcacgctgattctgatggtcgtgtgctcggtcgcgtccgggctctcgttcgggagctcggccaagggcgtcgtgtctacgctctgcttcttccggttctggctcggcttcggcatcggcggcgactacccgctcagcgccaccatcatgtcggagtacgcgaacaagaggacgcgcggggccttcatcgccgccgtgttcgccatgcaggggttcgggatcctcttcggcgccatcgtcgcgctcgcggtgtcggcggggttccggcacgcgtacccggcgccgtcctactccgacaaccacgccgcgtcgctcgtcccgcaggccgactacgtgtggcgcatcatcctcatgttcggcaccgtcccggcggcgctcacctactactggcggatgaagatgcccgagacggcgcggtacacggcgctcatcgcccgcaacgcgaagcaggcggcggccgacatgtccaaggtgctgcacacccagattgaggagagcgcggaccgcgccgagacggtggccgtcggcggcgagagctggggcctcttctcgcgccagttcctgcgccgccacggcctccacctcctcgccaccaccagcacgtggttcctcctcgacatcgccttctacagccagaacctgttccagaaggacatcttcagcaaggtcgggtggatcccgccggcgaagaccatgaacgcgctcgaggagctctaccgcatcgcccgcgcccaggcgctcatcgcgctctgcggcaccatcccgggctactggttcaccgtcgcattcatcgagatcatgggcaggttctggatccagatcatgggcttcgccatgatgacggcgttcatgctcggcctcgccatcccgtaccaccactggacgacgccggggcaccacaccggcttcatcgtcatgtacggattcaccttcttcttcgcgaacttcgggccaaacagcaccaccttcatcgtgccggcggagatatacccggcgcggctccggtcgacgtgccacggcatctccgccgccgccgggaaggccggcgccatcatcggagcgttcgggttcctgtacgcggcgcaggaccagcacaagcccgagcctgggtaccccagggggatcggcatcaagaacgcgctcttcgtgctcgccggcacaaacttcctcgggacgatcatgacgctgctcgtgccggagtccaagggcatgtcgctcgaggttatctcgcaggaggtcgccgacggcgacgacgaggaggcggcctacccgaagtaa

SEQ ID NO: 2 - DNA, 1587 bp, Oryza sativa
atggcgggatcgcagctcaacgttttggtcaagctcgaccaggccaagacgcaatggtaccatttcatggcgatcgtcatcgccgggatggggttcttcacagacgcctacgacctcttttgcatcgcgctggtcaccaagctgttgggtcgcctttactacaccgacatcaccaagcccaacccagggacactcccgcccaacgtgtcgtccgccgtcaccggcgtcgcgctctgcggcacgctcgccggccagctcttcttcggatggctcggcgacaagctcggccgcaagagcgtctatggattcacgctcatcctcatggtcgtgtgctccatcgcctccggtctctcgtttggccacacgcccaagagcgtgattgccacgctctgcttcttccgcttctggctcggcttcggcatcggcggcgactacccgctcagcgccaccatcatgtcggagtacgcgagcaagaagacccgcggggccttcatcgccgccgtgttcgccatgcaggggttcgggatcctcttcggcgccatcgtcgcgctcgtcgtctcggccggcttccgtcacgcgtacccggcgccgtcgtacgcccagaaccccgccgcgtcgctcgcgccgcaggctgactacacgtggcggctcatcctcatgttcggcaccatcccggctgggctcacctactactggcgcatgaaaatgcccgagacggcgcggtacacggcgctcgtcgcccgcaacgccaagcaggcggcggctgacatgtccaaggtgctccacgccgagatcgaggagcggccggaggtggtcgagagccaggtggtcgccggggagacctggggcctcttctcacggcagttcatgaagcgccacgggatgcacctcctggcgaccaccagcacgtggttcctgctcgacatcgccttctacagccagaacctgttccagaaggacatcttcagcaaggtcgggtggataccgccggcgaagaccatgaacgcgctcgaggaactctaccgcatctcccgcgcccaggcactcatcgcactctgcggaaccatcccgggctactggttcaccgtcgccttcatcgacatcgtcggcaggttctggatccagatcatgggcttcttcatgatgaccgtgttcatgctcgcgctcggcgtgccctacgaccactggacgcacccggcgcaccacaccggcttcgtcgtgctctacgcactcaccttcttcttcgccaacttcgggcccaacagcaccaccttcatcgtgccggctgagatcttcccggcgaggctccggtcgacgtgccacggcatctccgccgcgtccggcaaggccggcgcgatcatcggcgcgttcgggttcctgtacgctgcgcaggaccagcacaatcccgacgcgggatactcccgcggcatcggcatccggaacgcgctcttcgtgctcgccggcacaaacttcctcggtatgctcatgacgctgctggtgccggagtccaagggcttgtcgctggaggagatgtccaaggacaacgtcgtcgacgagaccgcccaagaagcgatcgcccaagcgtga

SEQ ID NO: 3 - DNA, 1581 bp, Oryza sativa
  misc_feature (386)..(386): n = any nucleotide
atggcggacgggcagctcaaggtactgacgacgctggaccatgcgaggacgcagtggtaccatttcatggcgatcgtgatcgccggcatgggcttcttcaccgacgcctacgacctcttctgcatctccctcgtctccaagctgctcggccgcatctactacaccgacctcgccggggacaaccccggcagcctgccgcccaacgtgtcggcagcggtgaacggcgtcgcgctgtgcggcacgctcgcggggcagctcttcttcgggtggctcggcgacaagctcgggcggaagagcgtgtacggcttcacgctcgtgctcatggtggtgtgctccgtcgcgtcgggcctctcgttcgggcgcacggcgaagggcgtcgtcgccangctctgcttcttccggttctggctcggcttcggcatcggcggcgactacccgctgtcggcgacgatcatgtcggagtacgccaacaagaggacgcgcggggcgttcatcgccgccgtgttcgccatgcaggggttcggcatcctgttcggcgccatcgtggcgctcgtcgtgtcggccgggttccggaacgcgtacccggcgccgtcgtacgccgacggccgcgcggcgtcgctggtgcccgaggccgactacgtgtggcggatcatcctcatgttcggcaccgtcccggcggcgctcacctactactggcgcatgaagatgccggagacggcgaggtacaccgcgctcatcgcgcgcaacgccaagcaggccgccgccgacatgtccaaggtgctcganacggagatccaggaggacncggaacgcgccgaggcggttgccgccggcggcgccggcaacgagtgggggctcttctcgcggcagttcgtgcggcggcacggggtgcacctggtggcgacgacgagcacgtggttcctgctcgacatcgcgttctacagccagaacctgttccagaaggacatcttctccaaggtcgggtggatcccgccggcgaggacgatgaacgccgtcgaggaggtgttccgcatcgcgcgcgcgcaggcgctcatcgcgctgtgcggcaccatcccgggctactggttcaccgtcgcgttcatcgacgtcgccggacgcttcgccatccagctgatggggttcgccatgatgaccgtcttcatgctcggcctcgccgcgccgtaccaccactggacgacgccggggaaccacaccggcttcgtcgtcatgtacggcttcaccttcttcttcgccaacttcggccccaacgccaccaccttcatcgtgccggcggagatctacccggcgaggctgcgttccacgtgccacggcatctcggcggcggcggggaaggccggcgccatcgtcggcgcgttcgggttcctgtacgcggcgcaggacccgcacaagccggaggccgggtacaagccaggcattggcatccggaacgcgctgttcgtgctcgccggcacgaanttcctcggcatgctcatgacgctgctggtgccggagtcgaagggcatgtcgctggaggaggtgtccaaggagaacgtcgccgacgacgaggaggcgacggcgtag

SEQ ID NO: 4 - DNA, 1617 bp, Oryza sativa
atggccggcgagctcaaggtgctgaacgcgctcgactcggcgaagacgcagtggtaccatttcacggcgatcgtgatcgccggcatggggttcttcaccgacgcctacgacctcttctccatctccctcgtcaccaagctgctcggccgcatctactacttcaacccggcgtccaagagccccggctccctcccgcccaacgtctccgccgccgtcaatggcgtcgccttctgcggcaccctcgccggccagctcttcttcggttggctcggcgacaagatggggcgcaagaaggtgtacggcatgacgctcatgctcatggtcatctgctgcctcgcttccggcctctcgttcgggtcgtcggcgaaaggcgtcatggccacgctctgcttcttccgcttctggctcggcttcggcatcggcggcgactacccgctctcggcgaccatcatgtcggagtacgctaataagcgcacccgtggagcgttcatcgccgccgtgttcgccatgcagggcttcggcaacctcaccggcggcatcgtggccatcatcgtgtccgccgcgttcaagtcgcggttcgacgcgccggcgtacagggacgaccggaccggctccaccgtgccgcaggccgactacgcgtggcgcatcgtgctcatgttcggcgccatcccggcgctgctcacctactactggcggatgaagatgccggagacggcgcgctacaccgcgctggtcgccaagaacgcgaagcaggccgccgcggacatgacgcaggtgctcaacgtcgagatcgtggaggagcaggagaaggctgacgaggtcgcgcggcgcgagcagttcgggctcttctcccgccagtttttgagacgccatgggcgccacctgctgggcacgacggtgtgctggttcgtgctggacatcgccttctactcgtcgaacctgttccagaaggacatctacacggcggtgcagtggctgcccaaggcggacaccatgagcgccctggaggagatgttcaagatctcccgggcacagacgctcgtggcgctgtgcggcaccattccgggctactggttcaccgtcttcttcatcgacatcatcggccgcttcgtcatccagctcggcggcttcttcttcatgacggcgttcatgctcggcctcgccgtgccgtaccaccactggacgacgccggggaaccacatcggcttcgtggtcatgtacgccttcaccttcttcttcgccaacttcgggcccaactccacgaccttcatcgtgccggcggagatcttcccggcgaggctgcgttccacctgccacggcatctcggcggcggcggggaaggccggcgccatcgtcgggtcgttcgggttcctgtacgcggcgcagagcacggacgcgagcaagacggacgccggctacccgccgggcatcggcgtgcgcaactcgctcttcttcctcgccggatgcaacgtcatcgggttcttcttcacgttcctggtgccggagtcgaaggggaagtcgctggaggagctctccggcgagaacgaggacgatgacgatgtgccggaagcgcccgcgacggccgatcaccggactgcgccggcgccgccagcttga

SEQ ID NO: 5 - DNA, 1638 bp, Oryza sativa
  misc_feature (612)..(612): n = any nucleotide
atggttcaggatcgcaaggtgctcgacgcgctggacacggcgaagacgcagtggtaccacttcacggcggtggtgatcgccggcatgggcttcttcaccgacgcctacgacctcttctccatctccctcgtcaccaagctgctcggccgcatctactacttcaacccggcgtccaagagccccggctccctcccgcccaacgtctccgccgccgtcaatggcgtcgccttctgcggcacgctcgccggccagctcttcttcggctggctcggcgacaagatggggcgcaagaaggtgtacgggatgacgctcatgctcatggtcatctgctgcctcgcctccggcctctcgttcgggtcgtcggcgaaaggcgtcatggccacgctctgcttcttccgcttctggcttgggttcggcatcggtggcgactacccgctctcggcgaccatcatgtcggagtatgctaacaagcgtacccgtggcgcgttcatcgccgccgtgttcgccatgcagggcttcggcaacctcaccggcggcatcgtggccatcatcgtgtccgccgcgttcaagttgcggttcgacgcgccggcgtacagggacgaccgggccggntncancgctgactacgcgtggcgcatcgtgctcatgttcggcgccatcccggcgctgctcacctactactggcggatgaagatgccggagacggcgcgctacaccgcgctggtcgccaagaacgacaagaaggcagccgccgacatggcgcgcgttctcaacgtcgagctcgtcgacgagcaggagaaggcagcggcggcgacggcggcggctgcggaggaggaggcagcacggcgcgagcagtacgggctcttctcccgggaattcgcacggcgccatggccaccacctgctgggcacgacggtgtgctggttcgtgctggacatcgcctactactcgcagaacctgttccagaaggacatctacacggcggtgcagtggctgcccaaggcggacaccatgagcgccctggaggagatgttcaagatctcccgggcacagacgctcgtggcgctgtgcggcaccatcccgggctactggttcaccgtcctcttcatcgacatcgtcggccgcttcgccatccagctcggcggcttcttcctcatgacggcgttcatgctcggcctcgccgtgccgtaccaccactggacgacgccggggaaccacgtcggcttcgtggtcatgtacgccttcaccttcttcttcgccaacttcgggccaaactccacgaccttcatcgtgccggccgagatcttcccggcgaggctgcgttccacctgccacggcatctcgtcggcggccgggaagatgggcgccatcgtcgggtcgttcgggttcttgtacgccgcgcagagcaccgacccgagcaagacggacgccggctacccgcggggcatcggcgtgcgcaactcgctgttcctgctcgccggatgcaacgtcgtcggcttcttgttcacgttcttggtgccagagtcgaaggggaagtcgctggaggagctctccggcgagaacgaaatggaggctgagccggcggcagcaactaactcctacaggcagaccgtccctgacagcggacagtccgagtaa

SEQ ID NO: 6 - DNA, 1605 bp, Oryza sativa
  misc_feature (1173)..(1173): n = any nucleotide
atgggcggcggcggcggggagcagcagcagcttgaggtgctccacgccctggacgtggccaagacgcaatggtaccatttcacggccatcgtggtggccggaatggggttcttcaccgacgcctatgacctcttctgcatctccctcgtcaccaagctgctgggccgcatctactaccgcgtcgacgggtccccgtcccccggcacgctccccccgcacgtctccgcctccgtcaacggcgtggccttcgtgggcacgctctcagggcaactcttctttggctggctgggcgacaagctcggccgtaagcgcgtctatggcatcaccctcatgctcatggtgctctgctccctcgcctccgcgctctcctttggccacaccccgacctccgtcatggccaccctctgcttcttccgcttctggctcggcttcggcatcggcggcgactacccgctctccgccaccatcatgtccgagtacgccaacaagaagacgcgtggcgccttcatcgccgccgtcttcgcgatgcagggcttcggcatcatcaccggcggcctcgtcgccatcctcgtctccgcctccttcagggccgccttcccggcgcctccctacggcgaggaccccgtggcctccacgccgccgcaggccgacttcgtgtggaggatcatactcatgctgggcgcgctgccggcggcgctcacctactactggcgcaccaagatgcccgagacggcgcgctacacggcgctcgtggccaacaacgccaagcaggccgcggccgacatgtccaaggtgctgcaggtggtggagatgcgtaatattggtaataatggtggcagcaggaggccgttcgggctgttctccggcgagtttgtccggcggcacgggctgcacctggtgggcacgtcggcgacgtggttgctgctggacattgcgttctacagccagaacctgttccagaaggacatattcagcgcggtggggtggatccccaaggcggcgacgatgagcgcgctggaggagctgttccgcatcgcgcgggcgcagacgctgatcgcgctgtgcgggacggtgcccggctactggttcaccgtcgcgctcatcgacgtggtgggccgtttcaagatccaggccgttggcttcttcatgatgaccctcttcatgctcaccctngccctgccgtaccaccactggacggcgccggggaagaaccacgtcggcttcctgctgctctacggcctcaccttcttcttcgccaacttcggccccaactccaccaccttcatcgtgccggcggagattttcccggcgcggctgcgggccacgtgccacggcatctcggcggcgtcggggaagctgggcgccatcgtcggatccttcgggttcctgtacctggcgcagagccccgaccggagcaagacggagcacgggtaccctccgggcatcggcgtccgcaactcgctcttcctgctcgccgcctgcaacctgctgggcctgctcttcaccttcctcgtgccggagtccaaggggaagtcgctggaggagatgtccggcgacgccgaggcccaggaggaggcgccgccgcccctgcaaactgtactgtag

SEQ ID NO: 7 - DNA, 1581 bp, Oryza sativa
atggcgggcgatcagatgcacgtgctctccgcgctggacagcgccaagacgcagtggtaccacttcaccgccatcgtcatcgccggcatgggcttcttcaccgacgcctacgacctcttctgcatctccctcgtcaccaagctcatcggccgcgtctactacaccgccgacggcgcgtccaagccgggcagcctgccgcccaacgtctcggcggccgtgaacggcgtcgccttcgtcggcacgctcacggggcagctcttcttcgggtggctcggcgacagggtcggccggaagagcgtctacggcatgacgctgctcttgatgatcatctgctccgtcgcgtcggggctgtcgttcggggacacgccgacgagcgtcatggccacgctctgcttcttccgcttctggctcggcttcggcatcggcggcgactacccgctcagcgccaccatcatgtcggagtacgcgaacaagcggacgcgcggggcgttcatcgccgccgtgttcgcgatgcaggggttcgggatcctcgccggcggcgcggtggcgatcgggatcaccgcgatcttcaggagccggttccccgcgccgccgttcgccgccgacccggcggcgtccaccccgccccaggccgactacgtgtggcggctcatcctcatgttcggcgcgcttcccgcggcgctcaccttctactggcggatgaggatgccggagacggcgcggtacaccgccatcgtcgccaagaacgcggagcgcgccgcggccgacatgtccaaggtgctccaggtgaagatcacggcggagcaggcggagatggcctcgccggtggacaagcccttcaccagcaagcccttcggcctcttctccggcgagttcgcgcggcgccacgggttccacctcctgggcacgacgtcgacgtggctcctcctggacatcgcctactactcccagaacctgttccagaaggacatcttcagcgccatcgggtggatcccggaggcgaagacgatgagcgcgctggacgagctgtaccacatcgcgcgcgcgcagacgctgatcgcgctgtgcgggacggtgccgggctactggttcacggtggcgctgatcgacgtggtcgggcggttcaagatccaggcggcggggttcttcgtgatgacggcgttcatgctggcgctggcggtgccgtacgaccactggacggcggcggggaaccagatcgggttcgtggtgctgtacgcgctcaccttcttcttcgccaacttcgggccgaacgcgacgacgttcatcgtgccggcggagatatacccggcgaggctgcgcgcgacgtgccacgggatatcggcggcgtcggggaaggtgggcgcgatcgtcgggtctttcgggttcctgtacctggcgcagagccccgtcccggccaaggcggcggcgcacggctacccgccgggcatcggcgtccgcaactcgctcttcgcgctcgccggctgcagcttgctcggcttcctcctcaccttccttgtgccggagcccaagggcaagtcgctcgaggagatgtcacgggagaacgaggtcggccagccgtga

SEQ ID NO: 8 - DNA, 1626 bp, Oryza sativa
  misc_feature (444)..(444): n = any nucleotide
atggcgcggcaggagcagcagcagcacctgcaggtgctgagcgcgctggacgcggcgaagacgcagtggtaccacttcacggcgatcgtcgtcgccggcatgggcttcttcaccgacgcctacgacctcttctgcatctccctcgtcaccaagctgctcggccgcatctactacaccgacctcgccaaggagaaccccggcagcctgccgcccaacgtcgccgcggcggtgaacggagtcgcgttctgcggcacgctggcggggcagctcttcttcgggtggctcggcgacaagctcggccggaagagcgtgtacgggatgacgctgctgatgatggtcatctgctccatcgcgtcggggctctcgttctcgcacacgcccaccagcgtcatggcgacgctctgcttcttccggttctggctcggattcggcatcggcggcgantacccgctgtcggcgacgatcatgtcggagtacgccaacaagaagacccgcggcgcgttcatcgccgccgtgttcgcgatgcaggggttcggcatcctcgccggcggcatcgtcaccctcatcatctcctccgcgttccgcgccgggttcccggcgccggcgtaccaggacgaccgcgcgggctccaccgtccgccaggccgactacgtgtggcggatcatcctcatgctcggcgccatgccggcgctgctcacctactactggcggatgaagatgccggagacggcgcgctacaccgccctcgtcgccaagaacgccaagcaggccgccgccgacatgtccaaggtgctccaggtcgagatccaggaggagcaggacaagctggagcagatggtgacccggaacagcagcagcttcggcctcttctcccgccagttcgcgcgccgccacggcctccacctcgtcggcaccgccacgacatggttcctcctcgacatcgccttctacagccagaacctgttccagaaggacatcttcaccagcatcaactggatccccaaggccaagaccatgtcggcgctggaggaggtgttccgcatcgcgcgcgcccagacgctcatcgccctgtgcggcaccgtcccgggctactggttcaccgtcttcctcatcgacatcgtcggccgcttcgccatccagctgctagggtttttcatgatgaccgtgttcatgctcggcctcgccgtgccgtaccaccactggacgacgaaggggaaccacatcggcttcgtcgtcatgtacgccttcaccttcttcttcgccaacttcggccccaactccaccaccttcatcgtgccggcggagatcttcccggcgaggctgcgttccacctgccacggcatctcggcggcggcggggaaggccggcgccatcatcggatcgttcgggttcctgtacgcggcgcaggacccgcacaagcccgacgccgggtacaaacccgggatcggggtgaggaactcgctgttcgtgctcgccggatgcaacctgctcgggttcatctgcacgttcctcgtgccggagtcgaaggggaagtcgctggaggagatgtccggcgaggcggaggacgacgacgacgaggtggccgccgccggcggtggcgccgccgtgcggccgcagacggcgtag

SEQ ID NO: 9 - DNA, 1560 bp, Oryza sativa
  misc_feature (855)..(855): n = any nucleotide
atggcgccgaggatacgggtgctcgcggcactggaccaggcgcggacgcagtactaccacttcaaggccatcgtcatcgccggcatgggcctcttcaccgactcctacgacctcttctgcatctcgccggtcatgaagatcttcgggcgggtgtactacgcgccatcgggatcagtagacggcagcggcagcggccccggggtgacgccgccggccgtcgtgtcggccaccgtcggcgtggcgctgctgggcgcggtggccgggaacgtggtcctctgcttcttccgcttcctcctcggcgtcggcgtcggcggcgactacccgctgtcggccaccatcatgtcggagttcgccaaccggggcacccgcggcgcgttcatcgccgccgtgttctccatgcaggggttcggcatcctcgtcagctccgccgtcaccatggccgtcgccgccgccttcgaccactacaccggctaccccgcgccgctcgacacgccggagtgcgccgacctcgcctggcggatcatactcatggccggcgccgtccccgccgcgctgacgtactactggaggatgtccatgccggagacagccaggtatacggcgctggtggagcgcgacgtggtgaaggccaccaacgacatcggccgcgtcctggccgacctcgacctggccgccgtcgccgaggaagaggtggcggcggcggcgttgagcccgcctccgcatgggagagacctgttcgcgtgcgcggcggcgtggttcctcctcgacatcccctactacagcagcacgctgttccagtcgcagatctaccggccatggttcccgccggcggcgaaggtgaacgcgttccaggangcgttcaacgtggccaagttccaggccgtcatcgccgtcgcctccaccatcccgggctacttcgccgccatgctcctcatcgagcgcgccggccggcggcgtctccagatggccgggttcctcctcatggccgtcttcctcttcgcgctcgcgggtccctacgatggatactggcgcgaccacgccaagaccgccggctacatcgtgctctactcgctcaccttcttctccgccaacctcgggcccaacaccaccaccttcatcctgccggcggagctcttcccggcgcgcttccggtcgacctgccacggcctgtccggagcggcggggaagctcggcgcgctcgtcggctccatcggcttcctgtgggcgtcgcagcagaaggacggcgcggcggccgggcacctgccgggcatcggcatgatgtacgcgctgttcgttcttggcgggatctgcctgctcgggctggcgctnacctacgcgttcacgccggagacgatgacgcggtcgctggaggagaacgagagcagcgtgcaggcgcagagccaggtcggcgacggcggcagcgatgccggcaatggcagtgatgggctgcgcttccacgaactaaatgttctcatggaggcggcgaccaagagccctgtgtccatggcgagctcgcacctcagcatgtcgcctatccttccgcaccggatgtctctatga

SEQ ID NO: 10 - DNA, 1650 bp, Oryza sativa
  misc_feature (219)..(219): n = any nucleotide
atggctccgatcggcgtgctcacggcgctggaccaggcgcggacgcagtactaccacttcaaggccatcgtcatcgccggcatgggactcttcaccgactcctacgacctcttctgcatcgcgcccgtcatgaagatcgtgggccgggtgtactactccgacggcggcgcccgtccgggggttacgccgccggccgtcgtgtcggcgaccgtcggagtngcgctgctgggcgcggtgatcgggaacgtggtgttcggcgcgctgggcgaccgcgtggggcggcgccgcgtctacggcgcgtgcctgctgctgatgctccgtcgggagcggcttctccgtgtgccggacgcgccggtgcgcgctcgccagcctctgcttcttccgttcctcctcggcgtcggcgtcggcggcgactacccgctgtcggccaccatcatgtcggagttcgccaaccggcgcacccgcggcgcgttcatcgccgccgtgttctccatgcaggggttcggcatcctcgccagctccgcggtcaccatggccgtcgccgccgccttcgaccactacaccggctaccccgcgccgctcgacacgccggagtgcgccgacctcgcctggcggatcattctcatggccggcgccgtccccgccgcgctgacgtactactggaggatgtccatgccggagacggccagatatacggctttggtggagcgcgacgtggtgaaggccaccaacgacatcggccgcgttctggccgacctcgacctgggcgccgtcgccgaggaggaggtggcggcggcgttgagccggcctccgccgccgccgcgcccttcgtacggcctcctctctcggcgcttcgtccggcagcacgggcgggacctgttcgcgtgcgcggcggcgtggttcctcctcgacatcccctactacagcagcacgctgttccagtctcagatctaccggccgctgttcccggcgccggggctcatcaacgcgttccaggaggcgttcaacgtggccaagttccaggccgtcatcgccgtcgcctccaccatcccgggctacttcgtcgccgtgctcctcatcgaccgcgtcggccggcggtgcctccagatggccgggttcctcctcatggccgtcttcctcttcgcgctcgcgggtccgtacgacggatactggcgcgaccacggcgcgcacgccggctacatcgtgctctactcgctcaccttcttctccgccaacctcgggccgaacaccaccaccttcatcctgccggcggagctcttcccggcgcgcttccggtcgacgtgccacggcctctccggcgcggcggggaagctcggcgcgctcgtcggctccatcggcttcctgtgggcgtcgcagcagaaggacggcgcggcggccgggcacctgccgggcatcggcatgatgtacgcgctgttcgttcttggcgggatctgcctgctcgggctggcactcacctacgtgttcacgccggagacgatgatgcggtcgctggaggagaacgagagcgacagggcgcagacccaggtcggcgacggcggcagcgacaccgaggcggccaagagcccggcgtccatggcgagctcgcacctcagcatgtcgcccatccttccagcccgggtctccgtgtga

SEQ ID NO: 11 - DNA, 1668 bp, Oryza sativa
  misc_feature (1362)..(1362): n = any nucleotide
atggcggacgcggacgggggctcgaacctggcggtgctggacgcgctggactcggcgcgcacgcagatgtaccacatgaaggcgatcgtgatcgccggcatgggcttcttcaccgacgcctacgacctgttctgcatctccacggtgtccaagctgctcggccgtctctactaccagcccgatggctcgacggacagtaagccaggcgctctgtccaagaccgccaacaacatggtcatcggcgtcgcgctcgtcggcacgctcatgggccagcttgtcttcggctacttcggcgacaagctcggccggaagcgcgtctacggcgtcaccctcatcctgatggccgcctgcgccatcggctccggcctgtcgttcggcagctcgcgcaaggcagtcatcggcacgctgtgcttcttccgcttctggctcggcttcggcatcggcggggactacccgctgtcggccaccatcatgtccgagtactcaaacaagaagacgcgcggcgcgttcatcgccgcggtgttcgccatgcagggcgtcggcatcatcttcgcggggctcgtgtccatgatcgtctctagcatcttcctcacctacaacaaggcgccgtcgtacaaggggaaccatgacctctcgaggcagatgcccgcggctgactacgtgtggcgcatcgtcctgatgatcggcgcgttcccggcgttggcgaccttctactggcggatgaagatgccggagacggcgaggtacacggcgatcatcgatggcaacgcgaagcaggcggcgaacgacatgcagaaggtgctgtcgatcgagatagaggccgagcaggagaagctggccaagttcaacgcggccaacaactacccgctcctgtcgatggagttcgcccggcgccacggcctgcacctcatcggcacgacgaccacgtggttcctccttgacatcgccttctacagccagaacctgacccagaaggacatcttcccggctatgggcctgatcagcggcgctgccgaagtcaacgctctcacggagatgttccagatatccaaggcctcgttcctcgtcgctctcctcggcaccttccccggctactgggtcaccgtcgctctcatcgacaagatgggcaggtacatgatccagctgatcggtttcttcatgatgtccatgttcatgctggcgatgggcatcctgtacgactacctcaaaacccatcacttcctgttcgggctcctgtacgcgctcactttcttcttcgccaacttcgggccgaacagcaccaccttcgtgctgccggccgagctgttcccgacgcgcgtgcgctccacctgccacgccatcagtgccgcggcgggcaaggcnggcgccatcgtcgcggccttcggcattcagaagctcacgtacaactctcaagtcaaaagcatcaagaaggcgctcatcatcctctccatcaccaacatgctcggcttcttcttcacgttcctcgtcccggagaccatgggtcggtcgctcgaggagatctccggcgaggacggcaacaccggcgccggtggcggcggcgcccctgccgctgccaatgccggcgttggcgtgagcgcttcggatgtgagcagggacgagaagttccctgcttcaagcaccgaatggcagacatccatgcacgcatga

SEQ ID NO: 12 - DNA, 1626 bp, Oryza sativa
  misc_feature (188)..(188): n = any nucleotide
atgggaaggcaggaccagcagctgcaggtgctgaacgcgctcgacgcggccaagacgcaatggtaccacttcacggcgatcatcgtcgccggcatggggttcttcaccgacgcctacgacctcttctgcatctcgctcgtcaccaagcttctcggccgcatctactacaccgaccccgccagccccanccccggctcgctgccgcccaacatcgccgccgcggtgaatggcgtcgcgctctgcggcaccctctccggccagctcttcttcggatggctcggcgacaagctcggccgcaagagcgtctacgggatgacgctgctgctcatggtgatttgctccatcgcctcagggctctccttctcgcacacgccgacgagcgtcatggccacgctctgcttcttccgcttctggctcggcttcggcatcggcggtgactacccgctgagcgccaccatcatgtccgagtacgccaacaagaagacccgcggcgcgttcatcgccgccgtcttcgccatgcaggggttcggcatcctcgccggcggcgttgtcacgctcgccatgtccgcggggttccaggccgcgttcccggccccagcgtacgaggtcaatgccgctgcgtccaccgtgccgcaggccgactacgtgtggcgcatcatcctgatgctcggtgcgctgccggccatactgacgtactactggcggatgaagatgccggagacggcgcggtacacggcgctcgtcgccaaggacgcgaagcaggcgtcgtcggacatggccaaggtgctgcaggtggaaatcgaggtggaggaggagaagctccaggacatcacgaggggcagggactacggcctcttctcggcgcggttcgccaagcgccatggcgcgcacctcctgggcacggcggcgacgtggttcctcgtcgacgtcgcgtactacagccagaacctgttccagaaggacatcttcaccagcatccactggatccccaaggcgcgcaccatgagcgagctcgaggaggtgttccgcatctcccgcgcgcagacgctcatcgcgctctgcggcaccgtgccgggctactggttcaccgtcttcctcatcgacatcatcggccgcttcaagatccagctcctcggcttcgccgggatgacggcgttcatgctcggcctcgccatcccgtaccaccactggaccatgcctggcaaccaggtcatcttcgtcttcctctacggcttcaccttcttcttcgccaactttgggccgaacgcgacgacgttcatcgtgccggccgagatcttcccggcgcgtctccggtcaacctgccacggcatctccgccgcgtccggcaaggccggcgcgatcatcggagcattcggtttcctctacgcggcgcagccacaggacaaggcgcatgtcgacgccggctacaaacctgggattggcgtgcggaacgcgctcttcgtgctcgccgggtgcaacctcgttgggttcctcatgacatggatgctcgtgccggaatcgaaagggaagtcgctggaggagatgtccggcgaggccgacgacgaggaagcttctgccaacggcggtgccaccgccgtcaactcgtccggagttgagatggtgtaa

SEQ ID NO: 13 - DNA, 1464 bp, Oryza sativa
atggccggcaaccagcagctgcgggtgctgcacgccctggacattgcaaggacacagctgtaccatttcatcgcgatcgtgatcgccggtatgggcttcttcaccgacgcctacgacctcttctccatctcccttgtcgccgacctcctcggccacgtctactaccacggcgagctgccccggaacatccacgccgccgtcaccggcatcgcgctttgtggcaccgtccctggacagctcgtcttcggctggctaggcgacaaaatgggccggaagcgtgtctatggaatcaccctcctcctcaatggttgtctcctctctcgcctccggactctccttcacatcggtggggattacccactctccgccaccatcatgtcagagtacgccaacaagaggactcgtggtgccttcattgctgctgtctttgctatgcaaggtttcggcaaccttgctgctgggatcattggaatgatagtatctgcagctttcaagcattcatcggcatcaaagattgactatgcctggcggataatcctcatgtttggcgccattcctgctgctctcacttaccactggcgcatgaagatgccagaaacagcacgttacactgccctcatctctaagaatgcaaagaaggccgccaaggacatgtctgccgtgctcaatgtcaacataaccccggacgatgaagttatcaatgagcttgcaaggcaggacgagtatggcctcttctcgttcgagtttctccaccgccatggcctccacctccttggcaccaccgtctgttggttcgtcctcgacgtcaccttctactcgctcaacattttcatgaagaatatcttcaccgaggtcggtcttcttcctcgtctagatagcgaataccaccataccctccagcggatgatcaccatgacagcagttcacacatttatctcgctttgcggtgcacttcccgggtacttcttcactgtcgccttcgttgatcgcatcggccgtgtcaagatccagctgattggattcaccatgatgactgttttcatgctttgtcttgcaattccttatgatcagtggctgaggcacaagaacaagtacggatttgcagtcatgtatggcctgaccttcttcttcgccaactttgggccaaatactacaaccttcataattccagctgagattttccctgcaaggctacggtccacatgccatgggatatctggtgcggttgggaagatcggtgcgattgtcggtgtgtttggtttcttgtacacagaataccatatcaggatctttctgtttgtccttattgggtgcaatcttgttggattcatcttcacccttctcttaccagagtcgaaagggaagtccctcgaggatctcactggggagattgaagagttccaggaggaagatgaaggaagtgaagttgctttgtctagacccatacatactgtgcctttatag

SEQ ID NO: 14 - protein, 535 aa, Oryza sativa
MAGGQLNVLSTLDQAKTQWYHFMAIVIAGMGFFTDAYDLFCISLVTKLLGRIYYTDDSKDTPGALPPNVSAAVTGVALCGTLAGQLFFGWLGDKLGRKSVYGFTLILMVVCSVASGLSFGSSAKGVVSTLCFFRFWLGFGIGGDYPLSATIMSEYANKRTRGAFIAAVFAMQGFGILFGAIVALAVSAGFRHAYPAPSYSDNHAASLVPQADYVWRIILMFGTVPAALTYYWRMKMPETARYTALIARNAKQAAADMSKVLHTQIEESADRAETVAVGGESWGLFSRQFLRRHGLHLLATTSTWFLLDIAFYSQNLFQKDIFSKVGWIPPAKTMNALEELYRIARAQALIALCGTIPGYWFTVAFIEIMGRFWIQIMGFAMMTAFMLGLAIPYHHWTTPGHHTGFIVMYGFTFFFANFGPNSTTFIVPAEIYPARLRSTCHGISAAAGKAGAIIGAFGFLYAAQDQHKPEPGYPRGIGIKNALFVLAGTNFLGTIMTLLVPESKGMSLEVISQEVADGDDEEAAYPNTPFVLFVD

SEQ ID NO: 15 - protein, 520 aa, Oryza sativa
MAGSQLNVLVKLDQAKTQWYHFMAIVIAGMGFFTDAYDLFCIALVTKLLGRLYYTDITKPNPGTLPPNVSSAVTGVALCGTLAGQLFFGWLGDKLGRKSVYGFNAHPQWSCAPSPPSVIATLCFFRFWLGFGIGGDYPLSATIMSEYASKKTRGAFIAAVFAMQGFGILFGAIVALVVSAGFRHAYPAPSYAQNPAASLAPQADYTWRLILMFGTIPAGLTYYWRMKMPETARYTALVARNAKQAAADMSKVLHAEIEERPEVVESQVVAGETWGLFSRQFMKRHGMHLLATTSTWFLLDIAFYSQNLFQKDIFSKVGWIPPAKTMNALEELYRISRAQALIALCGTIPGYWFTVAFIDIVGRFWIQIMGFFMMTVFMLALGVPYDHWTHPAHHTGFVVLYALTFFFANFGPNSTTFIVPAEIFPARLRSTCHGISAASGKAGAIIGAFGFLYAAQDQHNPDAGYSRGIGIRNALFVLAGTNFLGMLMTLLVPESKGLSLEEMSKDNVVDETAQEAIAQA

SEQ ID NO: 16 - protein, 526 aa, Oryza sativa
  X_region (129)..(129): Xaa = any naturally occuring amino acid
MADGQLKVLTTLDHARTQWYHFMAIVIAGMGFFTDAYDLFCISLVSKLLGRIYYTDLAGDNPGSLPPNVSAAVNGVALCGTLAGQLFFGWLGDKLGRKSVYGFTLVLMVVCSVASGLSFGRTAKGVVAXLCFFRFWLGFGIGGDYPLSATIMSEYANKRTRGAFIAAVFAMQGFGILFGAIVALVVSAGFRNAYPAPSYADGRAASLVPEADYVWRIILMFGTVPAALTYYWRMKMPETARYTALIARNAKQAAADMSKVLXTEIQEDXERAEAVAAGGAGNEWGLFSRQFVRRHGVHLVATTSTWFLLDIAFYSQNLFQKDIFSKVGWIPPARTMNAVEEVFRIARAQALIALCGTIPGYWFTVAFIDVAGRFAIQLMGFAMMTVFMLGLAAPYHHWTTPGNHTGFVVMYGFTFFFANFGPNATTFIVPAEIYPARLRSTCHGISAAAGKAGAIVGAFGFLYAAQDPHKPEAGYKPGIGIRNALFVLAGTXFLGMLMTLLVPESKGMSLEEVSKENVADDEEATA

SEQ ID NO: 17 - protein, 541 aa, Oryza sativa
MAGELKVLNALDSAKTQWYHFTAIVIAGMGFFTDAYDLFSISLVTKLLGRIYYFNPASKSPGSLPPNVSAAVNGVAFCGTLAGQLFFGWLGDKMGRKKVYGMTLMLMVICCLASGLVVRASWPRSASSASGSASASAADYPLSATISMSEYANKRTRGAFIAAVFAMQGFGNLTGGIVAIIVSAAFKSRFDAPAYRDDRTGSTVPQADYAWRIVLMFGAIPALLTYYWRMKMPETARYTALVAKNAKQAAADMTQVLNVEIVEEQEKADEVARREQFGLFSRQFLRRHGRHLTARASDVLGSTVLFVLDIAFYSSNLFQKDIYTAVQWLPKADTMSALEEMFKISRAQTLVALCGTIPGYWFTVFFIDIIGRFVIQLGGFFFMTAFMLGLAVPYHHWTTPGNHIGFVVMYAFTFFFANFGPNSTTFIVPAEIFPARLRSTCHGISAAAGKAGAIVGSFGFLYAAQSTDASKTDAGYPPGIGVRNSLFFLAGCNVIGFFFTFLVPESKGKSLEELSGENEDDDDVPEAPATADHRTAPAPPA

SEQ ID NO: 18 - protein, 545 aa, Oryza sativa
  X_region (204)..(206): Xaa = any naturally occuring amino acid
MVQDRKVLDALDTAKTQWYHFTAVVIAGMGFFTDAYDLFSISLVTKLLGRIYYFNPASKSPGSLPPNVSAAVNGVAFCGTLAGQLFFGWLGDKMGRKKVYGMTLMLMVICCLASGLSFGSSAKGVMATLCFFRFWLGFGIGGDYPLSATIMSEYANKRTRGAFIAAVFAMQGFGNLTGGIVAIIVSAAFKLRFDAPAYRDDRAXXXADYAWRIVLMFGAIPALLTYYWRMKMPETARYTALVAKNDKKAAADMARVLNVELVDEQEKAAAATAAAAEEEAARREQYGLFSREFARRHGHHLLGTTVCWFVLDIAYYSQNLFQKDIYTAVQWLPKADTMSALEEMFKISRAQTLVALCGTIPGYWFTVLFIDIVGRFAIQLGGFFLMTAFMLGLAVPYHHWTTPGNHVGFVVMYAFTFFFANFGPNSTTFIVPAEIFPARLRSTCHGISSAAGKMGAIVGSFGFLYAAQSTDPSKTDAGYPRGIGVRNSLFLLAGCNVVGFLFTFLVPESKGKSLEELSGENEMEAEPAAATNSYRQTVPDSGQSE

SEQ ID NO: 19 - protein, 534 aa, Oryza sativa
  X_region (391)..(391): Xaa = any naturally occuring amino acid
MGGGGGEQQQLEVLHALDVAKTQWYHFTAIVVAGMGFFTDAYDLFCISLVTKLLGRIYYRVDGSPSPGTLPPHVSASVNGVAFVGTLSGQLFFGWLGDKLGRKRVYGITLMLMVLCSLASALSFGHTPTSVMATLCFFRFWLGFGIGGDYPLSATIMSEYANKKTRGAFIAAVFAMQGFGIITGGLVAILVSASFRAAFPAPPYGEDPVASTPPQADFVWRIILMLGALPAALTYYWRTKMPETARYTALVANNAKQAAADMSKVLQVVEMRNIGNNGGSRRPFGLFSGEFVRRHGLHLVGTSATWLLLDIAFYSQNLFQKDIFSAVGWIPKAATMSALEELFRIARAQTLIALCGTVPGYWFTVALIDVVGRFKIQAVGFFMMTLFMLTXALPYHHWTAPGKNHVGFLLLYGLTFFFANFGPNSTTFIVPAEIFPARLRATCHGISAASGKLGAIVGSFGFLYLAQSPDRSKTEHGYPPGIGVRNSLFLLAACNLLGLLFTFLVPESKGKSLEEMSGDAEAQEEAPPPLQTVL

SEQ ID NO: 20 - protein, 526 aa, Oryza sativa
MAGDQMHVLSALDSAKTQWYHFTAIVIAGMGFFTDAYDLFCISLVTKLIGRVYYTADGASKPGSLPPNVSAAVNGVAFVGTLTGQLFFGWLGDRVGRKSVYGMTLLLMIICSVASGLSFGDTPTSVMATLCFFRFWLGFGIGGDYPLSATIMSEYANKRTRGAFIAAVFAMQGFGILAGGAVAIGITAIFRSRFPAPPFAADPAASTPPQADYVWRLILMFGALPAALTFYWRMRMPETARYTAIVAKNAERAAADMSKVLQVKITAEQAEMASPVDKPFTSKPFGLFSGEFARRHGFHLLGTTSTWLLLDIAYYSQNLFQKDIFSAIGWIPEAKTMSALDELYHIARAQTLIALCGTVPGYWFTVALIDVVGRFKIQAAGFFVMTAFMLALAVPYDHWTAAGNQIGFVVLYALTFFFANFGPNATTFIVPAEIYPARLRATCHGISAASGKVGAIVGSFGFLYLAQSPVPAKAAAHGYPPGIGVRNSLFALAGCSLLGFLLTFLVPEPKGKSLEEMSRENEVGQP

SEQ ID NO: 21 - protein, 541 aa, Oryza sativa
MARQEQQQHLQVLSALDAAKTQWYHFTAIVVAGMGFFTDAYDLFCISLVTKLLGRIYYTDLAKENPGSLPPNVAAAVNGVAFCGTLAGQLFFGWLGDKLGRKSVYGMTLLMMVICSIASGLSFSHTPTSVMATLCFFRFWLGFGIGGDYPLSATIMSEYANKKTRGAFIAAVFAMQGFGILAGGIVTLIISSAFRAGFPAPAYQDDRAGSTVRQADYVWRIILMLGAMPALLTYYWRMKMPETARYTALVAKNAKQAAADMSKVLQVEIQEEQDKLEQMVTRNSSSFGLFSRQFARRHGLHLVGTATTWFLLDIAFYSQNLFQKDIFTSINWIPKAKTMSALEEVFRIARAQTLIALCGTVPGYWFTVFLIDIVGRFAIQLLGFFMMTVFMLGLAVPYHHWTTKGNHIGFVVMYAFTFFFANFGPNSTTFIVPAEIFPARLRSTCHGISAAAGKAGAIIGSFGFLYAAQDPHKPDAGYKPGIGVRNSLFVLAGCNLLGFICTFLVPESKGKSLEEMSGEAEDDDDEVAAAGGGAAVRPQTA

SEQ ID NO: 22 - protein, 582 aa, Oryza sativa
  X_region (104)..(104): Xaa = any naturally occuring amino acid
MAPRIRVLAALDQARTQYYHFKAIVIAGMGLFTDSYDLFCISPVMKIFGRVYYAPSGSVDGSGSGPGVTPPAVVSATVGVALLGAVAGNVVFGALGDRVGRRRXYGACLLLMVCSSVGSGLSVCRXRRCALASLCFFRFLLGVGVGGDYPLSATIMSEFANRGTRGAFIAAVFSMQGFGILVSSAVTMAVAAAFDHYTGYPAPLDTPECADLAWRIILMAGAVPAALTYYWRMSMPETARYTALVERDVVKATNDIGRVLADLDLAAVAEEEVAAAALSPPPVTTPPPPRPSYGLFSRRFVRQHGRDLFACAAAWFLLDIPYYSSTLFQSQIYRPWFPPAAKVNAFQXAFNVAKFQAVIAVASTIPGYFAAMLLIERAGRRRLQMAGFLLMAVFLFALAGPYDGYWRDHAKTAGYIVLYSLTFFSANLGPNTTTFILPAELFPARFRSTCHGLSGAAGKLGALVGSIGFLWASQQKDGAAAGHLPGIGMMYALFVLGGICLLGLAXTYAFTPETMTRSLEENESSVQAQSQVGDGGSDAGNGSDGLRFHELNVLMEAATKSPVSMASSHLSMSPILPHRMSL

SEQ ID NO: 23 - protein, 549 aa, Oryza sativa
  X_region (73)..(73): Xaa = any naturally occuring amino acid
MAPIGVLTALDQARTQYYHFKAIVIAGMGLFTDSYDLFCIAPVMKIVGRVYYSDGGARPGVTPPAVVSATVGXALLGAVIGNVVFGALGDRVGRRRVYGACLLLMLRRERLLRVPDAPVRARQPLLLPFLLGVGVGGDYPLSATIMSEFANRRTRGAFIAAVFSMQGFGILASSAVTMAVAAAFDHYTGYPAPLDTPECADLAWRIILMAGAVPAALTYYWRMSMPETARYTALVERDVVKATNDIGRVLADLDLGAVAEEEVAAALSRPPPPPRPSYGLLSRRFVRQHGRDLFACAAAWFLLDIPYYSSTLFQSQIYRPLFPAPGLINAFQEAFNVAKFQAVIAVASTIPGYFVAVLLIDRVGRRCLQMAGFLLMAVFLFALAGPYDGYWRDHGAHAGYIVLYSLTFFSANLGPNTTTFILPAELFPARFRSTCHGLSGAAGKLGALVGSIGFLWASQQKDGAAAGHLPGIGMMYALFVLGGICLLGLALTYVFTPETMMRSLEENESDRAQTQVGDGGSDTEAAKSPASMASSHLSMSPILPARVSV

SEQ ID NO: 24 - protein, 555 aa, Oryza sativa
  X_region (454)..(454): Xaa = any naturally occuring amino acid
MADADGGSNLAVLDALDSARTQMYHMKAIVIAGMGFFTDAYDLFCISTVSKLLGRLYYQPDGSTDSKPGALSKTANNMVIGVALVGTLMGQLVFGYFGDKLGRKRVYGVTLILMAACAIGSGLSFGSSRKAVIGTLCFFRFWLGFGIGGDYPLSATIMSEYSNKKTRGAFIAAVFAMQGVGIIFAGLVSMIVSSIFLTYNKAPSYKGNHDLSRQMPAADYVWRIVLMIGAFPALATFYWRMKMPETARYTAIIDGNAKQAANDMQKVLSIEIEAEQEKLAKFNAANNYPLLSMEFARRHGLHLIGTTTTWFLLDIAFYSQNLTQKDIFPAMGLISGAAEVNALTEMFQISKASFLVALLGTFPGYWVTVALIDKMGRYMIQLIGFFMMSMFMLAMGILYDYLKTHHFLFGLLYALTFFFANFGPNSTTFVLPAELFPTRVRSTCHAISAAAGKXGAIVAAFGIQKLTYNSQVKSIKKALIILSITNMLGFFFTFLVPETMGRSLEEISGEDGNTGAGGGGAPAAANAGVGVSASDVSRDEKFPASSTEWQTSMHA

SEQ ID NO: 25 - protein, 541 aa, Oryza sativa
  X_region (63)..(63): Xaa = any naturally occuring amino acid
MGRQDQQLQVLNALDAAKTQWYHFTAIIVAGMGFFTDAYDLFCISLVTKLLGRIYYTDPASPXPGSLPPNIAAAVNGVALCGTLSGQLFFGWLGDKLGRKSVYGMTLLLMVICSIASGLSFSHTPTSVMATLCFFRFWLGFGIGGDYPLSATIMSEYANKKTRGAFIAAVFAMQGFGILAGGVVTLAMSAGFQAAFPAPAYEVNAAASTVPQADYVWRIILMLGALPAILTYYWRMKMPETARYTALVAKDAKQASSDMAKVLQVEIEVEEEKLQDITRGRDYGLFSARFAKRHGAHLLGTAATWFLVDVAYYSQNLFQKDIFTSIHWIPKARTMSELEEVFRISRAQTLIALCGTVPGYWFTVFLIDIIGRFKIQLLGFAGMTAFMLGLAIPYHHWTMPGNQVIFVFLYGFTFFFANFGPNATTFIVPAEIFPARLRSTCHGISAASGKAGAIIGAFGFLYAAQPQDKAHVDAGYKPGIGVRNALFVLAGCNLVGFLMTWMLVPESKGKSLEEMSGEADDEEASANGGATAVNSSGVEMV

SEQ ID NO: 26 - protein, 487 aa, Oryza sativa
MAGNQQLRVLHALDIARTQLYHFIAIVIAGMGFFTDAYDLFSISLVADLLGHVYYHGELPRNIHAAVTGIALCGTVPGQLVFGWLGDKMGRKRVYGITLLLNGCLLSRLRTLLHIGGDYPLSATIMSEYANKRTRGAFIAAVFAMQGFGNLAAGIIGMIVSAAFKHSSASKIDYAWRIILMFGAIPAALTYHWRMKMPETARYTALISKNAKKAAKDMSAVLNVNITPDDEVINELARQDEYGLFSFEFLHRHGLHLLGTTVCWFVLDVTFYSLNIFMKNIFTEVGLLPRLDSEYHHTLQRMITMTAVHTFISLCGALPGYFFTVAFVDRIGRVKIQLIGFTMMTVFMLCLAIPYDQWLRHKNKYGFAVMYGLTFFFANFGPNTTTFIIPAEIFPARLRSTCHGISGAVGKIGAIVGVFGFLYTEYHIRIFLFVLIGCNLVGFIFTLLLPESKGKSLEDLTGEIEEFQEEDEGSEVALSRPIHTVPL

SEQ ID NO: 27 - DNA, 1695 bp, Oryza sativa
atgtctcaatcttcccctttcttctccgttgcccgagcacatgctggagcaggagggcgagctgcggctgccgctctcctgctccgtcaccctgttgctcagctgccacctcggattcatggcctgaggtactacccttcagccattgtgtcacctgccaagacactgaactcacacctgagccttccccatgccacaatatcctcctttgccaatgctgataatggctccagcgggcaagcagatgccacagagtctgaggaggagcagaacggggaatctgaactgtcggagatggccaaggcattccatatatcgccccggatggcaatgtcaatctcagtggtgattgcatttgcagcgctcactgtgccactggcgatgcagtcagtggtcttccatgggacgaacaaaatgaaggcactggcatatctgacactgctttcagggttctacatggcatggaacattggggcaaatgatgtggccaatgccatggggacgtcagtagggtctggtgctttgacactccggcaagcagtgcttactgcggcggtgctagagttctctggtgcattccttatgggcacccatgtcaccagcaccatgcagaagggcatcctagtcgcatctgtcttccaaggaaaggactctctgctcttcgctggattgttgtcatccctcgctgctgctggaacatggttgcaggttgcttcctcctacggctggcctgtttcaactacacattgtattgtcggggccatggttggttttgggatagtatttggaggcgtaaatgcagtgttctggagctccttggcaagagtatcttcatcatgggtcatttcaccattgatgggtgctgcagtctcatttattgtttacaagggtatacgaaggttcgtatatagtgcaccaaatccaggtcaggctgcagctgctgctgcaccaattgcagtttttactggtgttactgcaatatcatttgccgcttttcctctcagcaagactttttcaatcgctatcctgcaagcattagcatgtggcgcaataggagccgtaatcgttaacagagtgatccaaaaacagcttgaaaagatagcatctgctgataaggcaaatgctcagcaagttggatttctttcggacatagctggaccaacaggagctcaattgcagattgtatatggtgtatttggatacatgcaggtcttgtcagcatgcttcatgtcatttgctcatggaggcaatgatgtctccaatgccataggccccctggcggcagctttgtccattcttcaaggtgtagcaagcagtgctgagatagtcatacctactgaagtccttgcttggggaggttttggaattgttgcagggcttacaatgtggggttacagggtcatagcaacaattggcaagaaaatcaccgaactaacaccaactagaggttttgcagcagagtttgcagcagcttctgtagtattgtttgcatcaaaacttggattgccaatatctgctacacatacacttgttggtgcagtgatgggagtgggatttgcaagagggctcaatagggtcagagcagagacagtgcgtgaaattgtggcctcctggttagtcacaattccagttggtgctgtgttatctatcttctacacattgctcttcacaaagattctggcatactttatgtga

SEQ ID NO: 28 - protein, 564 aa, Oryza sativa
MSQSSPFFSVARAHAGAGGRAAAAALLLRHPVAQLPPRIHGLRYYPSAIVSPAKTLNSHLSLPHATISSFANADNGSSGQADATESEEEQNGESELSEMAKAFHISPRMAMSISVVIAFAALTVPLAMQSVVFHGTNKMKALAYLTLLSGFYMAWNIGANDVANAMGTSVGSGALTLRQAVLTAAVLEFSGAFLMGTHVTSTMQKGILVASVFQGKDSLLFAGLLSSLAAAGTWLQVASSYGWPVSTTHCIVGAMVGFGIVFGGVNAVFWSSLARVSSSWVISPLMGAAVSFIVYKGIRRFVYSAPNPGQAAAAAAPIAVFTGVTAISFAAFPLSKTFSIAILQALACGAIGAVIVNRVIQKQLEKIASADKANAQQVGFLSDIAGPTGAQLQIVYGVFGYMQVLSACFMSFAHGGNDVSNAIGPLAAALSILQGVASSAEIVIPTEVLAWGGFGIVAGLTMWGYRVIATIGKKITELTPTRGFAAEFAAASVVLFASKLGLPISATHTLVGAVMGVGFARGLNRVRAETVREIVASWLVTIPVGAVLSIFYTLLFTKILAYFM

SEQ ID NO: 29 - DNA, 2003 bp, Oryza sativa
  misc_feature (780)..(780): w = adenine or thymine
cgctaaaattaaaagttcggttgaaattggaacgatgtgacgaaaaagttgaaagtttgtgtgtagaaaagttttaatgggatggaaaagtcgaaagtttgaaaaaaaattacaactaaacatggcccaagttctttttttttaaaattcaataacaatagtttcttataatatgaaaagtagtagctttgctattagacctttgctattagtagtagcttgtttggcgccatcctcttgttgagcaatcattcttcagaattcatctgaagtagcgaatggcaacaaaggggtaaatcgttttgtttgtttagctcgaaggagtaaactttgatgtaaccacccatactcaggatgggtgtaggcagtgcagatggcctaacgcattccgacggcaacggagttctggcgaaggagagggctccgtgacgaggacgaggaaggagccatcagaggaggaagtcggggtggccaaggccgaggtggagcaaggagggatgtgcccctggcagcggcgaaaggcagaagttcggagtaggtcggcacaggcaggaggaggttggggaagtccggcgaaagtatcggccgtggagctatacgccggagaagggaaggtggccatggggccaccattagcaagtgggtatggctggtcagatttggctacgtttgcctggagcaacagcaaccaacagggacacaacgaggtgaagagttatcctcgaggcgtaaaaggtgaagatggcaattcgcggggagctatagctagccttctccagwacttctgtccatatcctgttctgcgtcacgagccatgcaaacaaacacacacatctctgcgtcattcgcggggagtagtttcgctcgctcgctagggcatgcatgcttgtcaagttgctcgcaagagttcatcgtcaagaagcttcgctggaaatttcgtcgacctcttctccgattgttgtagcgaaagggacatggtttgttctctagctgaagtttcagccccatgtcaattccatcaccaaaacgagatagcgacaagccagatcgtattagctaggcatgtgccatgtggaaaaccaagaaacaagagaatttaagacgggaaggcccaagtgtgtactgcaactactgttgcgagctgtgcgtccaccctaccgaaggacgcctatgaactggacttcattgagccagtagtgcagccagcccatgctctgcaaaatcctaccctcgcatacttcaacggcgatgacttccatatcaaagaaagaatgttcagtcagaactactccggctaacaagtacagtagtaataaccaattgaacaaacacggcattcagctgctgatttccaagcctatccatatactccctctgttttttaatagatgacgccgttgactttttcttacatatttgaccattcgtcttatccaaaaattttatgtaattgtataagatataaatcacacttaaagtattatgactgatataacaactcataataaaataaattataattacgtaaaatttttgaataagacgaatggttaaacatgtgagaaaaaatcaacggcatcatctattaaaaaacggagttagtacttggttcttgctcagcttgtcgctgtcttgtttcaacctgctaatgagaagcgattgtgttacgtactacacagtacagactacagactacaaagtatccatctcaacatgcttcgtcagtaattgatgaagatgtgccggttaattactgctccaaacgcgggaagaacggagaccaaaagacttccagttttgcagtccgccccctctactgtgcccctgctctgcttttgataagaacggcaaccattagagttgactagtttgagcattcaacactggacctgcgactgcgagtagtatatatctgcgtccagtagagaacagtcagttcagcggcaaagcaaaagaaaaagttagatcagaggagagctcagagctcaaagagttgggaagcc

SEQ ID NO: 30 - DNA, 1740 bp, Oryza sativa
  misc_feature (1687)..(1687): s = guanine or cytosine
gatgatcttttttcctcaagctgcaaagttataagctttgcttagtacagacaacatatcacaaaagaatagaaaattcttcacctaggcatctccatttatatctgactagatcgttgaatgttaattgaacagagaaatttcaggtttcgcactgttgagattgagaattcatgatttaccgctcagtaattgaaacttcaatagataggggtgtgtccatttttaatgcgtactgtactaaatagtagaagcacatgtttacacaactaatcttattggcgggtaataatattacgcatatgattcaacaaatattcagtagaacgtttcctgaatatgctaaaccgcaacactaattaatctagcatctctgctaacatggaactcagatgcaggagagagggttgcgtaccttgggaagattgaggatgggttgactccggagagagagcagaggggatcgctgtgtgctctgttagtttcctcatctctgttgccatggtgctgtatatatagccgcagcgcagtccggacaggatacagacagggcatggttgcagaaaaaagattttctttctactgcattgcatgtaccgtacatactgtagattgcaagcagatagtgacatatattgtctttaaaatgcccgtacagaagtcaaatgttattctatttgcaggatcgtggaatgtgaaattaactttcaactatctatgcatatcagcgtatggcctcccactgtttagaattctacggtgtcaggatcggctgggcagtgacaaagctatgcatgcaaatagggtcttggtcttgagcagtgatcatttggttaagtcctacaccatcagtttccgatttcatattataacatattataagtcgtttagccttttctctagttaaactttgataagtttgatcaaatttataaaaagattagcaacatctaaaatacttaactagtttaacattgaatacattttatattttcataatatgtttgttttgtgtttaaaatagtactatatttttctttaaacttagtcaaacttaaagaagggctgttcagattgtagccaagataaatttaaaattttggtagtttgacaatattactaaaattttggtacgatttcttatgtatttatcaaaatttagcaagaaactaaatagatacacatatttgacaactctataaaaaatggtatggtttgaaatagcatcaatctgaacaagcctgaagtttaattagaaaaagatcaaacgacatactccctccatctacttttgatagtcatatttcatcttgccacgcagaccaaggataagtaattctacttatcattcatttaaacatgctactagtcatttctcgtaaacaaacgattcattaatattaacatttctcaatgcccatgtagccaatcatgcgtggaagaatggggagtcatgcattaaatccaagaaagtcattaagagaataggttgttggattgaaatatgactatcaaaaataaattttttagatttggaaatatgactatcaaaagtagatggagggagtataatataaaacagaggaaacatccgggaagttccagaataaaatcttggcaattgcctgctgatgtcatttggtgtcttcaacagttcaactactaatcgaacgatggtgggaaaaasaaaaactcaaaaacttgggaaaagagatctaaattagaaaaattgtacctgtg

SEQ ID NO: 31 - DNA, 2010 bp, Oryza sativa
  misc_feature (501)..(501): k = guanine or thymine
ttcgattcaagctggtcaaaaaccgcaatggccatgtccattcttccacacctcgcatacatgtcaaggagtgcactccctacatagacatcatcatgccagtcatacttcaccgtgagcgcgtggatctgttccccgatgccactgctcgcactagcaccagccgccttgaggagactcgcaaacgtgaacccgtttggcttgaatctccctctcagcatcccagggagcagcccaagggcctcatccggcatgtcgttctgcgcatacccggcaatgagcgaggtccaagagcacatgtcgcgcgccggcattccgtcgaacacacgtcgcgcgtcggccacggcaccgcacttgcagtacaggtggatgagcgagttgtccaggaagacgctgccggcgaactgggagccggccaggtgtgcgtggatcgccctggcgtcgtccaggctcctgtaccgcgcgcaggcggtgatgagagagtggtagaggcgcggggtkgcggcgagctcgcgggcgtccacgctgtcgagcgtatcgcggatgatgccggtggaggcagcggcggggacggggatgtctgcgacggaggcggtgaggctgggggctgcggcgtagcgccggttggcgtctcggcgacggcgaaggcggtggaggaggaggagactcgggaacggctttctcatggagatgaggagaagccggtcggcgcaggtgttcgacgcgttgcctcggagagaaggcgaggatggaacgacccccccgccgacggcgacacgcgatgcctagccgcagggtcgtggcatggggccgcccaaaagtggatggggcggcgggttggtggtgctgccggcgaggaggggcaagcaagcggaggcggcggaggaagccgggcgccggggaggggaagggargggagggcgttcgaggagcggcggcggcgcggcgccgacgacggcgggagagcgtggcggcggcggtggtcggtgagcgcgatgggcggctgggtcggccgttggattggagtcttggagatcgaatggttgcggttgctcggctgctcgcttgggcagagtgttcgatgacgtttgcattgtgttcctttttcccgtttctcaatttttactttctcattttttatgggcaagttttacgaattgataaacggtgtgttattttctaaaaaatatatgtacaataaagttgttttaaaaaaattatattaatctatttctcagattttctttaagctaatactatctccggttttttaatatacgatgtcgatgactttttaataaacttttgactattcgtcatattcgatcctttttaaatatgaaaaaaattaaatcctgcttaaaggcttgataaatcaaataacaataaaataaatgataactacataatttttttaataagacgaatagtcaaacgtagcttaaaaagttaacggtgtcatatattaaaaaaccggatgtagtaattaattaataatgcgttaatccgtcactctattttgtgtgtaaagagggaagggtttctaactccttcagaaggatggagggattctaacatcaaagtttctaagtagataaataatgtgaagggttgtggctacacagggatgaagcaaagacgtataaaaattgaagcttggtcttctagttttctttgttagtcctctttctttcttgttttattgtgttcctcagctatgattctatatgtgaaggatatagagattggatttttatccattatcttaaaaaagagctactcctaccatactgtgaaaacaatggctgaaatcacacaaattgcatcagtacatcaggacctacgtgctatctaaagccagaccgttttgctgcattccatggaccatgcatgattaacctctggttctgggcacaaaagagtaaaaacttccaatggatattttacaaacaccaaacaagacagatacaaattatccccatattttattacagggtccagtaggatgac

SEQ ID NO: 32 - DNA, 2000 bp, Oryza sativa
  misc_feature (1199)..(1199): w = adenine or thymine
gtgcagtgtcataagaaaataaccagtcttctaacaaataggcgtacacagttaacttaattaaacaagtataattgtccaataaaacgagaacatagttgagtattacttctacacaccagttgcaaatagaatctttttttaatgaatatcaacccgacgtctgtatttcgcattgatatgcacaaacaaaaatccccgctcttaaaaatggtaccaaaagtcgtgttctcttaaaaaattaagaaatgtcaaccagtttgatactcacttcgtctcaaaatatataagcgattataggttttggagaacttatcccttcggaacacactttgtttacattttatccgatcctaccccatccacccacacgctatccaattactcgtcgaattgataatagtctttttctctcattctagcttgatctctctaaaagaccatgaatcccttgaatacggagataggtaaaccttgtcttgttttgactagacaaattattgattcctcgagcatctaaatggcacattaattggggacaaaaacgagatcgttgtatttgtctatctggccccacagtagaagaaacctgaatataaaccaatgatacaaaacaataaaaagtcaaattcaacatcactggtagtagatggataacatagataacacgtacgctgcccaaatttcgaagatattagtacgtgatttgtttgactaatcttattcttataggagtattgcattaggagagacaaacaaatcgaagcgatgacgtggctctttctagctagttttgtcatttgctccctataaataccatccatgtgtaccttgccatcggttcttcagagttacagtgctaacggcctgcagcagagtgcagtgactcccctgaagaaactggtatattaatatcaggtgtgtatatatttcacattttattctagtactactattaatgacatgtctatatatgtcaattttaagtacatacatgtatgggagattaaatttttgatatgttcacaagtttcttgctgatgaaacatgcgtcaaggcaggatgttgtgtaagggtgttaattactgattggtcattagttgccctcatgaatccatgaaaaagttcttcataaagtcatcacaaggaagagaccttttgtgctctctttacggcatgcaaaggtcacgaacagtttwacaaaaacaccttttgcatactagtctccctcctcgtcatacttcagcaaccacaagatttcttttctgaactttttactaatgaacattcagaaatttctgtgcaatattatctcatgaacctgaaccaaacgatgcttgagccacgaaatagtagaggagacaaagatatagtttcgtcaattcgagaagtttgtccggatactacggatgatagcggcagatttggactggttccatgaaagttgtacagtaaggtgcgaatcttgagttgcagagatgcacctggatccggctatctagcttcacgagaatcccatctctactctcctaaattgcccacgaaactgaatttatgtagggatttttagcgaaattcagacatttttcacggggatgggtcggggattgttgactgataaagctggatttgaagaaacaacaaaattttgatatatgataccttgaataaacgaggagtttctgaagtagtggcatggtctgttccagatgtctctctgaacttccgtttcagtttcagtggaccatattgttggtgaactgaaacgaatattatcttctcgtagccacgtgcattctgtagattttcttttgctcagttcgacacatagacatctgaggctaattagctctgttaatcgcgcggtttgtgtaattctcacaaataattagtttctcgttcattgcaaattgcagcgagattttgtcgaaataataaacttggtgttcagttattctctgcaaaaaattgcatattgcagagtagctgagattggcgcc

SEQ ID NO: 33 - DNA, 2003 bp, Oryza sativa
  misc_feature (1457)..(1457): y = cytosine or thymine
tttactttcgtatggttataattttgttgctacaaaccttacagtatatgagaaattataagttaaagattagttttagagaccataccaattttgacagtgccttatattatgggatagagtgggtatctaataaattgccgttgtgttgggttgtgaaattacccgatgttttaactaatttgatatttcaggaaaaatccacataaggtgagatggaaatatcccgttaactccaaattatttttaccaaaaagattaattaaactaacaagctttgatcgagctgggtgtttggtatatttgagtacttattatatttttatcgttttgaaagtacgttggcatgtttgctgtcttccgttgattcatgcaagatgccaagaaaacaaaatattttcttatatttaattttagtcgtcgtcaagctcaggtatagaatatcgtgatatgttggacctgttagatgtgtccaaatccacgcagatggaagtttggaaacacagtttcaagggtcattccagaagttcaaacaaccagccagctagtccctccatcctcaaatataaaatagtaccaaaatagtacccctccgtattttaatgtagaacgtcattaaccttttcactaacatttgatcatttttcttatttaaaaagtttatgtaattataatttattttattgtgatttaatttatcatcaaatattttttaagcataatataaatatttttatatttaataagacgaatggtcaaaaaaaatcaacggtatcattcattaaaatacagaagaagtatcttctaaaatttgagaggtcagttttttttagaactcatacccacaaacttaaacatttatgaggtacttttattattgttctttccccccttattcactggacaggaacattccagttgatggtttccttcaattaggggcatatcgtccaaaaaactataattgttttgattttccttcgtcttaatctctataagaaaatagaaatttttattttttttgggtggatccatatagtattatatataggtgatatatacataagcttctcgtgcacacacaaactaacataaaaatatcatcgaaattttcttaaaaaaaatatacatgtgcctcctatagtaatagagaatgaaaacacctatatagaaagtttcatcttcaaattcatcatttaaagaggaaaaaaacaaaattcttgtagttaatactcataaaactcaactttttttctctcttttttgcaggtataatataatgaacttacagttgagacatacatatgaataatattattgaaaatattttataaacttttctaaaattttcactcgaagtttgtgcacccaacatatgttctcttatataacatacctggtatgaataagcaactacattacaatagaacaacacaagtcaagattaatatatatayacatgaaatattcccactgttttctttatttaacattggaaagtataaaatagaactaaccaacgtcagaaaagtaaaatagagggaacatattaacctaaagatcagcatttaaaatatttttcgtgtattttcctcatttacagataattctacataacagtacactactatttagtgagtaatagttaatgatggctatatgatccagaggttcaaactgtatatgttctagaccattcacaagctgtgccaaaataccaatgagattatgacatgcatgcatatgcattgccctatagtatgatggtaaccatgcataccagccataacaaattaattttgtccatgcatgaacaaaccagaatgttccaggatttattctctagctaatacctgtcacttacaccaaattccttgcactatatatatagctacaataaatcatgcatgcatgccattgccattgaccatatgcaagcctagtgccataacaatttcacaataacttagagtattatatcaaagatagtcgccggcgccgcc

SEQ ID NO: 34 - DNA, 2023 bp, Oryza sativa
cgaaagtcgctattggggttgcacaagggatgagttcatcttcttctctcttattcctcctcctcatcaacctgtttctataggatgactaggagtttgtgggtggctagtagcttgcagttatggtaggacagagtgtctatgtcccgctgtagagctgtggtgacaggccacacataacagtgataggctcggcgttagttcttttctcaagttggtggttgggagtcgttagtttcatgctggtgagtttttcttttctttcctaattgtagcctcctagagttgtaatattgtatattttttcatgctatattaatatgaagcctcgcagctatctcatgtggtttgttcaaaaaaaataacatgatcaattgttgtttgttgtgtagtggtctctccggtgtcactatcggtgatttgacaaagacactcccaatgttgaatggtgtaaagatagtcatgtctccagtctttgcacatatatataatacatagagtcaaagtttaagatcaaaggtaaccttaataagcgattgaatgctctatgtgatggaaatgtgcatgacgtctatagtcctttctaagactctaagagtgagttacaaaccgcctttgctacctgtacgttgattgagttttagaaaaatcgtagttgattgatcaatcatcaatctgttgttgatctaagggctatagctctatcttgtacctcgagccaagagggagacatgataaggagagggatatgtcagagaggactcgcgacaacgtcctggtggagatgggcgggcggtgggtgggggtgagatcatgtggttaggttgcaagggtgatagatctagcagcgtgagccaccccgagatgaaggagaaaacacatatggatttgttgatgaagagggtgatggcagtgatgtggagggaaagaggacagggataggaaaggcttgtccagcctcgccggtgtcagcgctggcagtgcaaagaggaatgccatcacacgcgagtcaaagttgggacttagaacgttggtggcacaagagacgggatcaggagagggggaggctatgttaaaattagattgttacacaaataacaatgtaatctgagctatttaataaactatatataaaacatcaggcaactaatgaatggtcaagccgttatcaattattctggatggtaactccctgccttcgatgcaggacggagtgtagcgggggcccctgtccgaccatcccttgcgataagagtttttttctttctcagtaacttatatttcctgtatttgtgttttttgtttcttttttttccttgacccctagcaacggccctctgaccgtttgcgttgtgtaaccaaactctgttgtcttcttctaatatatttgactgtgcaatcatttagcgcgttcgcgagaaaaaaattggtttggatggttaaatcttactagaaaccagaacaagataggctacgcaattagcaatggatggttggcattaggtcatctaaggtcaaaccgacggagatacatttgctgcataaatagcaactagcgtaatatgtgatgtgatgtacttcctacgtcttataataagtttattttttagctacttatatttgtctagaaataagttaatttttagaataattatttgtatcggagtttatgaaagtaaaaagtaattgtatttggagtacataaagtgaggaagtattgagatttgataaagtagaggtattttagtttttttttattggtacgtatgagattagtgaaaaataaatttatttgggaagggatgtagtacctagctactcttgtaccagctagattaagtataaaccaaaaacaacgggagtagtagtagtaagaatgtaaaacggttccaacataacgcaacaacggaacggttggatattcggttgggaggagaagggagattccgtttgagtaggttataaatagcaaggaatacatacgtacgttccaaatcatcacaaagcaagcaattaagctggc

SEQ ID NO: 35 - DNA, 2135 bp, Oryza sativa
  misc_feature (1674)..(1674): r = adenine or guanine
gccggcgatgacgatggcggtgaagtggtaccactgcgtcttggcgctgtccagcgcggagagcacgtgcatctgatcgcccgccatggcggtgacgctgctgctccggcggtcaaaggctctctctctctctctctctcacacaaacaccttcctgcctcgtaaatggcggcgagggagtgagctacagagagatggaacaggtggttggttgctatgtatacgcgggtgacgcgagaatattccgagttcatatccttcatttttctcgggagaatgcaacgccttctccctcctttccagcaacaagttttgacgggctcgcatcgcaatcgcatggctctgcgtgttttggatccatatcccattagcttatcgccgtactacacacgagacgtgcaaacctgcagattttgttacttgttatgtatatactctgatgatcacatgatacacatgatatgtgtttgccgatacaattcgtaacaactctgcttccaaaattcggattgattgttgagacaaatctcgtagaacacggaacggagagagaggaagcaacggctaatgggccgggcgatccaaaacaaactggacctgaatgggccgcaccgtattccgtcgtgaaaccattgcgaaactcgaaaaaacatggcgtcttggctttgacttctcaccatagaagaaccttctcatggtcgtagcaagaaattccagtccaacgcggcttcgcttccggtgccgtgtcctccctagaaatcatcgtccctaaattggacaaaattacactgaactacgcagatccatcgttgcgaccacgcagaagatctccgtgctcgggtggcgcggcgggatggcgtcctcatcggcggcggcggcgacgtaccggtgcgcggagtgcggggcggacctgaacctgacggcggcgcagctatacccggagggggtctacttcgaggccggcaacaaggggacgctctccttctcgtgggtggacgagtcgcggctgcggttcgcgccggaggacaagatccgccccttcttcgagacgctcaactactgggggatccagcggaagcgcacccgcatcagctgcgacgcctgcggccgcctcctcggctacgtctacgacgacgggccgccggccatggacggcaccggccagttcgggatgggccccagccaggtcatccctcgccggccgaggtaccggatcaagaccaaggccgtcaccgtggtcaatcctccggctaccgcaccgtacagatgagatgaaaccttgtacatcaacatcgtagtagcgttcggtgttacaagtaggacatgtaaaacgtattccattgtgatcttggttcgccgttgttgcaaataaataaattgtagtagtactatgggatgatgaaaccgatgagaattatggtgtggtttctttgtaaatcaacgcaatttcttggtaaatccagtctccggtttgccacatactagtagcacggtggcacggtgcccatcggctggataccgacccaaccaggtcaagaattggggatctcacccaaatacccaactcaggagcaacgcaacggtaaatagacgggacgaggggaagaataacccagccatggccgtcgcagcacgcgcctcctcctccctcctccgcggcgtggccgtgttgargcggcggccgccggtccgatccatccacgaggggcccgacaccatcgacgagctgctcgaccgccacctcgccaagaagtccccctcctcctcttccggggcgctcgacggagacgcggcggacgccgargcgcgccgccggctgacgagctcgcggcgggargcgctggggctgtaccgggacatcctccgggtcacgcggctgttcgagtggcccgacgaccgcggcgtcccgtggcgcgaggtgctgcgcgccaacgcgcggcgggagttcgaggaggcccgcggcgagcgcgacccggaggtggtggcgcggctcctcatcggcggccgcgacgccgtgcagcaggcgctcgaccggctcgccgaggcgtccgccgcatggtcgaggcagaggaggccaagcgccgcggcggagcatagctgaagtgcggctacatgccgtttcgatgcgtttcata

SEQ ID NO: 36 - DNA, 2007 bp, Oryza sativa
  misc_feature (1283)..(1283): m = adenine or cytosine
cctgatttttcaatactccaagcaagtctaatacaataatgaatgaacaggaaaacaaacttgtcctttccattctccattaatgtttcagatatccatggctgcacgtcagcggatgaactatacttcattgaattcaaacctgaaattttttggttaataacaatgcaaagtcaaccacccgattaaccaacttgtggatcaaatcaacaattcccaaatttttggcagccgctctgcagcagcatatacaaaattctgaaaattgcaagtcttctcttctccccggcgcgtccatggctgacaggcctgcagtactacgttcatgcatgtgacattcaaaaaataacatttgatttgatgcctttccagaagattaaccgtaattaatccatgtatcggtcaatggagatgcatatcctcgttctgattaaaataaattggatctttgtaattatttacgcatgatgtcctagccttttggtcccaacacgtcaccagactacagtgcttaaaaaatacaataatgtgttcacagcttgagtgacaatgactagctagcagtcaaatggcccattttacaattatccatagctgtatggtctccaattaagtaagctcaacgcaatagtttagcttgatagttgataattagatagtactatctcctaaccaacacctcatgatcagtttacgttagtgtgaacgatctcccaggcaggcgcagtttcaggagaggatgatgccaatcaggatgattatattttgttgcaggttcaactgaatttgcttgttaattctgagatattcagtcatcaaaggacgctgtaaagtattgtatctgggttcacataagggtatcatatcttattatgggtacctttttttttttccgtagagagagatactgtgggaaccaaatttcagtgatgctggtcatcgtcagcaaccaaatttctcagaaatggctgcaaaaatgtaagacttcagcgacagacacagtgaaagttaatcagttgatacaggattttgctcggttgagaacagtggacgctggacactgcttcaaatcaatttagtgggctaacctcgaacggtccaattcacaggccagaaaagtccaacgtaccttttgggtgaactagttgacgagaattcgagcaacaattacgccgcagatctaagcttcggagcttagcgagcggtccagatctcacgtcagaaagttccataccgaaaactttttttttttttgagggaaatactgaaaactcttttaagcmcsaaggtagaagtccacccatttctcacattatcaactaaataattatgaaatttttttaaaattttgacaagatagattgatatgtaatatatcactctaaaaaatgcaagttgaaatacaacttctacaagttataaaaaaaacaacaaattaaactatatactacagtttaatttgttttttttataacttgtaaaagttaaatttgaacttgtatgtttgtgaagtgatatatttcatattaatcaattttatcatttttttataactgtttagttaacatgtaagaaacgagtgaacgttcactcgggagattaaaatccatcttcttcggtatactaaaagcttttagggtgaactgattgaaaaaaatcatacatacaataaatatattgtatatcgcatattaataagtcagcgtgcgaagtgcttctgggatgaaccgatcggtgacccgagcgcgtgcgttcgggcacctatcaaacccttgtacacaagcgcatggccccgcgtgagattcgtgccgtttttcttccctctccatattacagtgacaaccaagcaggcgtgccaaatccactggacacggccaagtcggctaccattcctgcgcctacatataccgcggcatctcccgcgccattgctgtgcccagagagctcgacacaaatacagggggactcgtcttcttccccgagctttgcgagcagagtcgttcagcc

SEQ ID NO: 37 - DNA, 1999 bp, Oryza sativa
  misc_feature (614)..(614): m = adenine or cytosine
agaacatgcggaatcgagtgcaacatcaatcgactagttaaggcagctgtacctgccaaatccaatgcaacattttgagactccaatgcagaaaaaataaggttggcatgccatagttataacaaacaactaacaagctttgctactccctccttctcaaattgatcatcatataatagaatccaaaatttctcaaattgatcatcatataaccatatggatatggatttcatcgaaatataataaatgcaacataggagaatgtgtgcatgcaagggtgttattggcatggtgttttaatttatgcatgcattgtgagaaaatatgtacatgatgtcttggttgatgcgatttaaattatctatggtcttggtgcatatataaacatatatatagtgatcaatttaggatagagagaatattttgcaagcgcctgaagatgccctcagtccctcactaattaactaaaaggataggctaaagcaaacaatggcatatgtaaggtttagcgtggcaaatacttgtgctaaccaaacatcttcttacttattaattggatcttgaaatgatactaatatgagttgtacctcttcttgcattgtgtcataaaaccmaagcttcagcacatcttcgttgtgcaaatatattatgctcgtgcgtgtggaaggtcacctaattaaccaaggtgctagggatacgcttctttttaagccagctagcactataggattgataatttatcatattattttggtgtttttagcatatcgaacttcgaaaatagaacaattttgaatacccattttttgttggtcgaatgaatataatagaacaattttgaatacccactttttgttggtccaattgggggaagctgtaggcgttcctcaggtcaatctattggggtgccacccacttgcatatatacaaagtttggcgctaaattgttcctgacacaatttaaaatccttcggagctctttaggattggaccagaacgtggcgcgccctaagtaacatcgggagtgtaatactaaatcttctagagacaaatgtataaacatatacttttatattccgtactactagtgtatattcaatagagattcactaattgccagtttgccacgttaaaaaaaaaaaagaagagggaaactcaacggctgccaagtgaggagtttaactggttatgtttctagtagtagaaccagctcatccagattcaaattttagatttggtatagatttagcatgggtgctcgtatttttggttaattattctttcaggagtaggcgacgtacttgtcgatagtgagacgcttgtggtgacttcaacaatttcaagatccaatctttcggagatattcatatgggtaggatgtgcatatgtgtgttcatagggacgagtgtgcatatgtgcgttcgtaggaacgagtgtgcgtggtatatcggcccagtcttttagaggtgttcatatagataggatatgcgtgtgtttacagtgcgcgcgttgtggacttatctgtttgtgctgtgttttaaaaaagaaagaaaaactagacggatgggttgctgggattctgggcgcagcgcggcaagccccaaacacgcgccctctccaaagtcgaaagtcgatcgcatacgaaagcgctgcagattggtacgcgagtgttggccattggccgtattttctttatccctctttattagtagcactccacataatgtatctgatgaagcgagaaatgggcggcatcttcctcggcatatctcccagttgtgtacggcgatgtcaactccctcacccatttctttaacccgtccacggctccacgtcgccgtcaccagtgagcaagctagctaccacctatcggctatcccgtcgtcgtctaacctccgcactccgccggcgacttctccctgtatatagatatcaaaccaccactcaaagaagctaaagaaccaacggttgctagtgctagctagctaggtcggcagca

SEQ ID NO: 38 - DNA, 1999 bp, Oryza sativa
  misc_feature (663)..(663): y = cytosine or thymine
ccgaccgggagtcatctcgaggatttggagatgttctgtgatatgtgatgatttggatttgggatttcttggcatgattaggggatggtgtgaaatcaatattcaatatgaaaaagagaaaaaaagaaaaaatatctttagtcccggttggtaataataaccgggactaaagatgctcccatctttattcccgggttgttgtttgaatttgaaccgggactaaagaaagcgatctttagtcccggatttgttgtcccggtacagaggggttacggaccgggactaaaaagcacttctccaccagtgttcgtctcgcaatttgcacgctaactgtgaaattagtttttttttaatatttcatactccgtacatgtgttcaaacgttcgatgtgacgggaaaaaaaatttgggaatgctatgtaccggaatcccgtccggacggaacccttacttattcaaagaggactgatacctgatcatgcacgtcccgagcagcagcagcagcctatgcaggacacggtgccgctccaacacacacgcatggccgacatgggcggcggcgctgcggcgccaacgcccgcaccgtcaacgcactaccccgcgaccgtgagctcctcggcggcgacgccggctgtggcgcccaggcccacaccatcgacgcaytgccccgcgaccgcgagctcctcgccggcgccggggaggagcctagcgcgracgcggcggcagataaggaagtagagcgcraaggtgctgccctcgccgccatcatcggcgcggagcacgacgagtacgtacctggctagctcgtcgaggcggtggaggatgcggccgccggcgacaagctccgacgtggcgtgaacggtggaggatgcggccgtcggcgacgagctcctgtgtggggcgtggatgccggaggatgcggccgctggcgacgagctccgacgtggcacggacggtggaggatgtggccgccggcaatgagctctggcatgggacgtggacggtgggaccaccggcgacgacgctttggtgcctagctcctgatatctagtaggtatcgtctgatacctcgcaagtatcaccttctacgtgataagaatcgcatgatatctgataggtatcatctgatacctcacaagtatcacctgatacgtggtagaatcgcatgatacctgataggtatcacccgataccggttaggtatcaggacctgatacctcgcaagtatcaccctctatgtggtaagaatagcatgatacctaataggtatcatatgatacctcacaagtatcacctgatatatggtagaatcgcttgatacctgataggtatcacctgataccggttaggtatcaggacctgatacctcacaagtatcaccctctacatggtaagaatcgcatgatacctgatagatatcatctgataccttgcaagtatcacatgatatgtggtagaaacgcatgatacatgttaggtatcagaacccgatacctaagcggtatcatcccgttcaaaaaacgggattccgttgtatagcagccccaaaaaaatttgccggtggatctaaacaccccctaaatcttaaccagaatatatggatgtgtcactttctggagacaaatgtgtatcaccagggtcctacactcgatcccaatttcccaacaccaaacaccagggtcgaacacgaaagcgctagcggcagattataatttgtacaacaccgtgggtaagtggccgtattttatcgcgtaatctatctgatcaagccaacgattagtggcatcttccttcggcatctcacttttgtacggtgatgtcaacgccccccggccatttctttaacccctcacgtcgccgtcaccactcaccaccaccatcgtcgtctacctcgccacctgtatatatacaaaaccagctaaaatatcatcaatccacggctgcaagctcgttcgtcggctgcttctgctagcgtgctcgctcgctcgctgcggcgaca

SEQ ID NO: 39 - DNA, 2149 bp, Oryza sativa
gctcaccagctagcaccgtcttcacctggctccggggatgcgtgcacctaacactacagactaataggggagtgatgggagagggcggagctttggtgtcgtcatcgatgtcttctcgtcttcagctgtcgttgctagtgaagatcttccaagttccaagtctcacttggcacatccctaccggtcacgtcctcttccctgaatgctccgaggtggccaccatcctcggctccgggtcaccaccaccatcgccctgtcggggccatctcgccactaccgctaccctcccgagctttactgttgttggaaggtgagagacgaagaagagggaagaaaaaaaagagagaggtgggagggggagggggaggtgaggaggaagataagacaaggaaatcaccaatatgtgaatcccacatgctaatatagtaagtcaactgcagtaaacctggcatctcaggagataccgcttcccaaattctcgaagtagttgatttacatcggtttttgaagttagggaaggcattatacccggttttgcggctgagggagcgattcattcaggagcaatagacgagggatgccaaatagacttgttcctattagaccgtaagattattgatgggacgaaaggccttgcatagatagtgatatctgagcccatgcaggggctatcaccaatgcagtgttgctagaaagtgctgtatcactactagcaaaataaaaagtttgtctgctaagagaaaaaaaatggacccatttaacatagttctagctctagataagtgggaccgtacaaattcttcatttttttagcgaagttagaagttagtagagttgctccacaaaattgtactacgggggtggagatgggtttttgctgcttcaccgctccactccatccaaaaaagctaactaacttttaatttttcatgtatttaccgaatatgccactgaactacccattcacaccctctctcatcccgttcttcctcccaactaggtggctgggtggcgaccggccgctgggactggtggcggcaagcgggttgcacagctgctaggcggcttggtggtgtgcgagggcagcaagggagagggcggtggcacgcgggctgagcatcgatcggcagggtcagcaggacaacgtgtgggtgacagggtgtgggatgagcgacgacggtgtcggttggttgggtggcatgcataggccgtaaccagtcgtggggaccagcggctattgggtgtcttggtggcgcacaagcgcaacagggcggcgcacgtgtgggcagcagtgcacaggctgagcggtgacgcgcgacggcggtggtggcaagaccgcgggacgccaaccaagtattcgatgaaatgagatgggatgtggtagcgtggagctaaccaaatctagccaaacatttttacaactaccttcagcttctattagagttggagtttaaaattaggagttgaaatttggaggcttaacaaatggggccgaaatatctgtttttaggcagacggcgtagcggtaaatcgatgatgtagacagttggaccgcaactgcaaggaaacaacactctgtcacacaggttccgccgtatggtaggaatcgccaatcatgattatctgccgaatatcctaccaaaaacgacgcatttccgtcctagttaacttcgatgctgctgcgctgcatcctccaggtaaagtaaaatcttgcaagggacaaaacacacaggctcttctcccatatccatcagctgcccaattgacgcatcactgagctgaattacacgcgcgcagggcaacgcagcatgattgatttcgtcattctcctctcgagacaaagaatccgatctcatctccgcacgagggacggccaactgcttccacccttcacaatgccgcctagacgcctagtgtacgcgccgaatatgctgccaaaccaacacgggacaatctcccgcgcttggcgacagcttctcctcggtgcagacgccctctcgttcatcgacctagcttgagggtgaaatcccagctataagatcgggcaaggcagcgagcagtttggtgatcagaggtagctagctagcctcgtagcagtgttccccggcggcgagagcggcaggagaggacgatcgacggcatcatcgga

SEQ ID NO: 40 - DNA, 2010 bp, Oryza sativa
gagcgggacgcggccgggttcgcgacggcgcacacgcaggctgggccggtgatgggctgcgtgggaggtgtgctgctgatgcatgggccaatgcgcgagatagctcggtcgcgtggtatgtgtcgggcctcggttcccacgggcttcgcacgcaggccggatacagaaacagatcgaatctcggaacaaaaacagagcgccacatatcactatatcagatgtcacggtggatttgccgccgcgcagatcgggatagctggcaggcattcgtacacccggatttctgctttgatatattctgttatcacagtatatccgagggcaccgtatctggtgcaaaccagggttattgcgcaaccttgcaaattttcaatcaagaccataggatgtccatccgatggctagggatataggtgggttacttttgcacttaaccgcccacactcatctatgctaatctgttttttatgcaaatcccccctaatcacagcgtccgtccgtccgcccttcccgcgtcgtttcgtttctacagcacacgcgattcgccgccgcccgctcgagtccccgccgcccgccagcgactcgccgacgccccttcgccacgccaccaccgccatgttgcaaaccctcccgttgtgaggtcacggggtagaccgtgccgctgccgccatggagtctggcacgcgcgacgtttcggattccaacgcgatagaccctgtgcaggaccagcgggcgggtgccgttgatcctatcgtcgctattgatcccgtctccgttgaggccgccgccatatatcatgctgtcattatcgtggacgcgggccaaacaaggtgtgaactatgaaccgtggaagtgtacttgctggaagtttattctgaacttgtatccctatacttgatgtattcctgaatgagacatgcttattttcgtattgtgaacattttaatcttaacctgtatgctcttgttatgggtagatgctgccaaaatttagtaaaaaggggtacatgctaccaaaatctgaactgcatcaagagtttcttatctaagacatgtttgccatcaatatatgacaatacatgaaactgaacctgtatcaaacctatcaagctgttgtgctctagcacatgtgcagaaaatgcagaacaagtagtttgacagtttgacagattacaaaatccgtgctggtttcagttcatctgtcatccgaatgcatgaagctgggcatatattcagtttgacagattgacacagaaaacagctggtagttcattcaggctattatacagttacacacccataggttccactgtcaccacaagcaagaaaaatgtttttttatagttcaacttgacttgactcaatctcattatcaaatgacagaacacaattccattgcatatttgcagtcacaatctttcagagtttcagattacaaaattagtacatcagtgtcaatctcagaacacgattccattgcatatcagctcggcgggccagcgaccgcgtcgagccggctgggcggggacgcggccgcggcggcgagcggcgacagcgggaggcgcagctcggcgggccagcgaccgcatcgagccggctgggcagggacgcggtcgcggcggcgagcggcgacggcgggagggcggaggacggcggccacatgaagcagggggggatttgtataaaaaacagattagcagagatgagtgcgggcgattaagtgcaaaagtagcctacctctgtccctagccatcggatgagcatcatgtagtcttgattgaaagtttgcaaggttgcacagcaactctggtttgcacgagatacggtgccgtatccgagggggtgctagtgtctgccaatggcgttgcatacgtatctcgtttgtataacggaatcgggctctttgcacggcacacggttcatctcgtactcgaactccgatctgttataaccatcgcgttggatcgtagcagcagccgccgacccaaaacgcaaacgcaaacgcgacgcc

SEQ ID NO: 41 - DNA, 2000 bp, Oryza sativa
ccattaatgattttaatttattccctaaataaatcatgacattgcatatgtaaactagcataatcgcatcatacgatctacacatgtaaactagacagtaaagcatgaacaggtcaaatctgcgaaacatgttaaatgcgtaccgagggttttggaaaaccggttgctcagtaggaataactcatggtacagtgcgttgacaacggcgcgcagcacatgagagaagaggaagacgacccgttgtggacgaacagggagtagtcgcgcaaagcacttcccaaaaaacttactgccaccttctcctggtgcaggacgtcgaagacgaaggttctggaaacctactctcccaatcgccggtgcatgcccccgagcgggatgaagtagactatgagtgatggcgcagtatagaggaacaggcaaaccctagattgatttcacatgttgcgtggaggcagcgattctacttatatagagatatcaggatgcttgatcaggacgcctgcacgatctctactccaaataaccgaatcgaataagtcgcgcgtaacttgtccggactccacgccgtttcacgcaccgtattattcggagcgtttccaaaaaaacaaatctgaattttctgcagcaaaacaaaactgcatgagggaccaattttatcggaccattcgacgcgtacgtcgtgcacgcgcgccactatgtctggccaggcgaggcgagcgagcgtacgtgtgtgttccccctgttctctccgcctgacatgcctcaagtggctaggagagcatcctcccttttaaggagggactaaactactcatacatgttatcccatgaggtgtggttttgtgattttttaaagaattaatcttcaaatgggttaaggcctatatattaattccaacaaagttgtcgataaagtttcctgtgtacaagttcatatttgtttgaattatatattatgaaaagctatataaagtataactatatggatatattctcatgaaaacattattgataccatttttgcaaatgaattatgaaatcttaaaatgtttgtacatccatacatcctaagaccagatctatctgtaaacggagcagtgtgttctgaactcgctcttacggaatctgtatcttcattagagaatattacacccagcaattcatcgggcacgtcagatgccaggatgattgaaacagacatgttttactttcttgttcctgcttgccgtccgggacttcaagtgcgacgactttgaataaattgcaaccagcaagttccagctaacttctgatctgaagaaccaagcacgtataccactagtattttccagtctacatttaatttgcactacctagttgctatcagttgttacactaaattcaagatttctacagatccaattatatctgatacaacaataagaatattcattcagttcaccatatgctagtgtcttgttgcacaagtacggacacatggagacaataaaccatttgtgactcccaagtggctgtcaggttcagcagattgtagaaatgaaattcataaagttcagggaactatctaaaaaaactgcggcaagtttttcaagggaaaacattcaaatctaactatagtataattataatatattttcactacaactatactatagctataatataaattgtatataagtattaaaataatgcctaacataatatatgcagattataatatagttacaatacagttacactaattatttacgtaaaaatactagtgtaattacattatagttatactataactatactatatttacatttgaaagtttttgcaaaaaaaaacctttgacagttttttagcaattccgttcagaaatcgagatggaaagcataaaaccttcgatatgacagagacagttgaattatgcttggaagcttatataacttagcaacttataatagcaaggacaggatcgaagaaattaaggaagcaagtcggatcggaaagcaaca

SEQ ID NO: 42 - DNA, 1999 bp, Oryza sativa
tctatgatcatatcatataaatttttaatctttggattaaaatccaatagacatagtaaaaggcaaaaagcaactaagaaacaccataatagatactaaattaccatatctttaagaaaaagaccaaatccaatataaatttcaaaacttacatgcgaagattcaaaaattacactgtaagtttcataaattacactataatttacaagaaaatacactgtaaatttgagtgagaaaatcgagtgagagataaagaaaaaatctttcaagtgagatatagcaaaatcgcagaacaattcgagtccaagattattattcattaaatggacttcactgagtcatttcttgtgaagggatttcactatgttattttgatcataaatctagagttaccacttttactccccataatttgattagactttcaaggaaatattgtacgtcaaaatttgatatctcaaaatcaaaccgataaataaacatggtttgcttaaaatataacaccacatttttcacgtaatttcaatcatttttttcctatgttattgcgctctctcccctatatatataagtatgcttctatctctcctgtttgcggccgtcgaacttgcaaatggctcagtggtgccctgcaatttggtcgagttacaaatctccatttggagctacaagtatactactactcagtatctttgtttttaagttatcgtacaaattcagaagcaacaccaaactgaaatcacaggaagattcttatacctaactacagattcgaaagtgaagtcggcaaaaggagaatcagccataagaaatcttgaaactgtgcacgctttaaagacacaatacttcatccacaacacacgacttgcagggaaaaaaaaatgtcgacctgagccctggccaactcgatgagcagaaaatgtgtctagtgcggtgtctgctaaatgataaattgtggccaatctctagtgatcgtagaagacaagatcaagtattccatacctgataaattgtggccaatctctagtgatcgtagaagacaagatcaagtattccatacctgatacagggacaaagacgctgattattcttctcttcagcatccggcctggagcacacaagttgattttctgttctgtggtccttggccagcagcgctccaatcgctactgccaaacgggaaccaccggtccatctcaccaacaaaaagaaacacgaagcaaacgcataagcatgcttacaatcttattcttggaccacagctgcgttcacttgttaaacatttattagtcgaacaagaggaaatatgcataccctgatacccatgagaagccagaccagtgttgacttcctctgcaattaaatagatttctgagcatggaaaatcttgattactaacaccgactgtatatctgaagagtcaccgtttgtgtgtcataagaacagtacgtttttctgtcataagaacagtacatataacatgagaacttcacatgtaactgtattagtttcatttatctgcacagtagatggcatcagctttagaaccacagacgcaagtatcttcagaagccttccatattaaactgatgcaagatgttaatgtcatgctaaatattgcactggagaatcaaaacaacagaaacagacaacagttagatctttaaaaaagacgatggagggaagggtggccctgatggtaacgcagactgaaaacagtcaagtggtcagctgcaaccccaggttatggattaggctggaatcttgctcgtatccccttccaccactcattccaagccaccactcgttcatctcagcccgagctgggccaccacagaaagcaagccctttcctccatccacaaaacaatgctcacttcattccacaaacacctatggccaccgcttctcattcaccttcctcctctcctcccccttaatctcatctttttcttctcggtgcttctctgctcaaacctgtactttgcttctgccactcc

SEQ ID NO: 43 - DNA, 22 bp, Oryza sativa
ggttctttcaaatccagggaaa

SEQ ID NO: 44 - DNA, 23 bp, Oryza sativa
ttttcagtcactcacgtcgagac

SEQ ID NO: 45 - DNA, 24 bp, Oryza sativa
acaaatgccatcaaatatgaacag

SEQ ID NO: 46 - DNA, 31 bp, Oryza sativa
ctaagtggcatttataatatcaacagtaacc

SEQ ID NO: 47 - DNA, 27 bp, Oryza sativa
ccatagaagagatccagagaagctgta

SEQ ID NO: 48 - DNA, 20 bp, Oryza sativa
caactggcggtttcttcgat

SEQ ID NO: 49 - DNA, 21 bp, Oryza sativa
tggaattaacgggtggatcac

SEQ ID NO: 50 - DNA, 24 bp, Oryza sativa
aaaatgtattcgtgccaaattgct

SEQ ID NO: 51 - DNA, 29 bp, Oryza sativa
aaaacctaagaagcactgtaaataaatcc

SEQ ID NO: 52 - DNA, 19 bp, Oryza sativa
tcgctttccgacgatgatc

SEQ ID NO: 53 - DNA, 23 bp, Oryza sativa
tgcatatcccagatgagcgtatc

SEQ ID NO: 54 - DNA, 32 bp, Oryza sativa
tcctgtacctaaaaagcaagtactaacatagt

SEQ ID NO: 55 - DNA, 28 bp, Oryza sativa
tgatttaagataaggattgaatgcacat

SEQ ID NO: 56 - DNA, 25 bp, Oryza sativa
aattttatggtcgaaactttttgga

SEQ ID NO: 57 - DNA, 18 bp, Oryza sativa
acgggacacgaccaagga

SEQ ID NO: 58 - DNA, 21 bp, Oryza sativa
cgcttccgtacgagtggtagt

SEQ ID NO: 59 - DNA, 19 bp, Oryza sativa
caggagaccgcccaagaag

SEQ ID NO: 60 - DNA, 22 bp, Oryza sativa
tgcgactgctgattcagtacgt

SEQ ID NO: 61 - DNA, 28 bp, Oryza sativa
ttctgctagtgtaccaaacaaaattaca

SEQ ID NO: 62 - DNA, 23 bp, Oryza sativa
tgctactgcccatgactaggatt

SEQ ID NO: 63 - DNA, 18 bp, Oryza sativa
ccgcccctgcaaactgta

SEQ ID NO: 64 - DNA, 20 bp, Oryza sativa
gcaagtcgctcgaggagatg

SEQ ID NO: 65 - DNA, 27 bp, Oryza sativa
agaaggcaaaagaaatgtgtgttaaat

SEQ ID NO: 66 - DNA, 26 bp, Oryza sativa
agaaaaacataggcttgtcatccttt

SEQ ID NO: 67 - DNA, 18 bp, Oryza sativa
atgtcgcccatccttcca

SEQ ID NO: 68 - DNA, 22 bp, Oryza sativa
gagaagttccctgcttcaagca

SEQ ID NO: 69 - DNA, 22 bp, Oryza sativa
aacgagatgacgaacacttgca

SEQ ID NO: 70 - DNA, 35 bp, Oryza sativa
ttagcataattcttttagtagttaaataggagatg

SEQ ID NO: 71 - DNA, 21 bp, Oryza sativa
tgtacagcccattgcacttca

SEQ ID NO: 72 - DNA, 23 bp, Oryza sativa
gggttcacaagtctgcctatttg

SEQ ID NO: 73 - DNA, 30 bp, Oryza sativa
caccaattcttcaagaacatatacacacat

SEQ ID NO: 74 - DNA, 28 bp, Oryza sativa
gatcgcccaagcgtgatgtcataaacat

SEQ ID NO: 75 - DNA, 29 bp, Oryza sativa
catttggacactgccattcacatcacact

SEQ ID NO: 76 - DNA, 26 bp, Oryza sativa
tactagtcgtcaacccaggcaacgca

SEQ ID NO: 77 - DNA, 28 bp, Oryza sativa
caacatggtgaaaatctgctggttccca

SEQ ID NO: 78 - DNA, 19 bp, Oryza sativa
tagcgctgtcgccgtctgc

SEQ ID NO: 79 - DNA, 22 bp, Oryza sativa
ctggccgacctcgttctcccgt

SEQ ID NO: 80 - DNA, 30 bp, Oryza sativa
tctgcaagaattattcctcaagtggccaaa

SEQ ID NO: 81 - DNA, 23 bp, Oryza sativa
cgtttccgctcctcctgccaatc

SEQ ID NO: 82 - DNA, 24 bp, Oryza sativa
agttcactcacacggagacccggg

SEQ ID NO: 83 - DNA, 23 bp, Oryza sativa
cgaatggcagacatccatgcacg

SEQ ID NO: 84 - DNA, 30 bp, Oryza sativa
ttcctatcacgctacaagtacgagcttcgc

SEQ ID NO: 85 - DNA, 29 bp, Oryza sativa
ttggtggcagatttctatccgtgacactc

SEQ ID NO: 86 - DNA, 27 bp, Oryza sativa
tcagcctatctctttgccattgcggaa

SEQ ID NO: 87 - DNA, 28 bp, Oryza sativa
cagaccagacaaccatagctccattggg

SEQ ID NO: 88 - DNA, 25 bp, Oryza sativa
gctcaccagctagcaccgtcttcac

SEQ ID NO: 89 - DNA, 25 bp, Oryza sativa
tcattggctatttgcgttaccttcc

SEQ ID NO: 90 - DNA, 25 bp, Oryza sativa
ccaaaacaaggagtacctatgcatc

SEQ ID NO: 91 - DNA, 25 bp, Oryza sativa
tccgatgatgccgtcgatcgtcctc

SEQ ID NO: 92 - DNA, 25 bp, Oryza sativa
ggagtggcagaagcaaagtacaggt

SEQ ID NO: 93 - DNA, 25 bp, Oryza sativa
gtacaggtttgagcagagaagcacc

SEQ ID NO: 94 - DNA, 620 bp, Musa acuminata
  misc_feature (14)..(14): n = any nucleotide
tttgcatacatgtntganagtaccacaagattggatcaccagattggggtgtgcttaacgaagcgcaggtcgaccgaggacggaggttaacgctaaacagggactgtcctgccgtaaggagcaggggaggaggtgtcctgctcctcttcgttctcgccggacatttcctccagcnatttgcccttcgactcgggcaccaggaaggtaaagataaggcncacaagattggatgccgcgagcacgaagagcgcgttgcggacgccgatgccggcagggtaccccttgtcccgcttcgtcggatcccggctctgcgccgcgtacaggaatccgaaggcgcccacgatggccccggccttgcccgcggctgccgagatgccgtggcatgtggaccgcagccgcgccgggaagatctccgccggcacgatgaaggtggtgctgttgggcccgaagttggcgaagaagaaggtgaacccgtacatgacgacgaagccgatgtggttgcccgaggtggtccagtgatggtaggggatggcgaggccgagcatgaacacggtcatgaagaagaagccatgagtggatggcgaaccggccatggtgtcatgaaggcgacgtgaaccagt

SEQ ID NO: 95 - DNA, 605 bp, Triticum aestivum
accatgatgaccattttcatgctagcaatcgaccataccgtacgactacttggtgaagccagggcatcacaccggctttgtcgtgctctacgggctcactttcttcttcgccaacttcggccccaacagcacaaccttcatcgtgccagctgagatcttccctgcgaggctccgatccacatgccacggtatctctgctgttaccggtaaggcaggcgcgatcatcggtgcattcgggttcctgtatgcgtcacaggaccagaagaagcctgagaccggctactcacggggaatcggcatgcgcaacgctctcttcgtgctcgcgggcacaaacttcctgggtctgctcttttcgctgctggtgccggagtccaagggcaagtcgctggaggagctctccaaggagaacgttggcgacgatgactccattgccccaactggtgtctagagacgtgcaggtgtacttgcacactcgtgcattatttgtgtgtttgatttgagtttctgtgttctgctgtttgttttgttcatgcaattttctgaacttttagtgttgtaatggcgtgacaatgtaatgcgtaaactgtaaagacaatgagtaaatt

SEQ ID NO: 96 - DNA, 900 bp, Triticum aestivum
cctagaggagctcaattgcagattgtatacggcgtatttggctacatgcaggtcctgtcagcatgcttcatgtcattcgctcatggaggcaatgatgtctccaatgccatagggcctctggccgcagctttgtctcttcttcaaggcgtagcaagcagcgctgagatagtcataccgactgaagtccttgcttggggtggttttggaattgtcgcagggcttacaatgtggggttatagagtcatagcaacaattggcaagaaaatcaccgaattaacacccacaagggggttcgcagcagagtttgccgccgcttctgtggtcttgtttgcatctaagcttgggttgccaatatctgctacacatacacttgttggtgcagtgatgggtgtgggatttgcaagaggtctcaacagagtcagagcagagactgtgcgtgaaattgtggtctcctgggtggtcacaattccagttggtgctttgctatcagtcttctatacattaatcttgaccaagattctgaaatactttatgtgagtgctttgtgagaaacagttgctgttttgagaggtccagccgttgcccctaaattttttatgctgtaaaaaaatgtgtaacagtgaatagatgagccagttccgggagtttccagcagtggaattgaaaagatttgtcagagcaaataatcattttcctggcaacaaagcacatgattaggataaaggatttatggtaaataatcatgtttgagactcccagcttcttcatcttttatctctaatgtacacctatatgactatctttatattttataccggaaggatggaatatatcaatttcttgtagagataatgaataaaattgatcatacataatagacacatggttcgtcatcacaac

SEQ ID NO: 97 - DNA, 597 bp, Triticum aestivum
ccggcaaccagatcgcttcgtcgtcatgtacccttcaccttgttcttcgcctacttcgccccgacgccaccaccttcgttgtgcctcccgagatcttcccggcgaggctgcgctccacgtgccacgggatctcgctcgccgcggggaaggccggcgccatgatcggcgcgttcgggttcctctacgcggcgcaggacccgcacaagcccgacgccgggtacaggccaggcatcggcgtccgcaactccctcttcgtgctcgccggggtcaacctgctggggttcatgttcaccttcctggtgccggagccaacgggaagtcgctggaggagatgtccggcgaggcccaggacgacgaggaccaggcacgcgccgccgccgccgtgcagccgtccacggcctagtaggccgccatatattccttcacaactcgtgcgtgctagtgaggcagctgcaggctgttgagctagtcaaagatccgtaatttgttgggtaatttgttttgtgatacgtacacgcataaaatactattacaactataggtttgcgggccacccaacctgcagggccggctcatgtatatgcccatgatatatac

SEQ ID NO: 98 - DNA, 764 bp, Triticum aestivum
tggtcacggtgcctgatcgacggatcgcgcttctggatccactggtcggcttcttcttcatgcggtgttcatctggggctcgccttcccgtaccaccactggacgacccgtggaaccacatcgggttcgtggtgctgtacgggctcaccttcttcttcgccacttcgggcccaactccaccaccttcatcgttcccgccgagatcttcccggcgaggctccggtcgacgtgccacggcatctcggcggcggccgggaagctgggcgccatcgtggggtcgttcgggttcctgtacctggcgcagaaccaggacccggccaaggtggaccacgggtacaaggccggcatcggggtgaggaactcgctcttcatcctcgccgcctgcaacttcctcggcatgggcttcaccttctgcgcgcccgagtccaacggcctctcgctcgaggagctctcccgcgagaacgaggaggaggcgccggacgccaggacggtgcccgtgtgagaaacacgtcgtcagatagtagtatattctgcgagcaggtttcgatggatgtatggatgttttttatggtcggagagcgttcaactcaggagtcgagtcgagtcgagtacaagaataatctatctaatcttgtgatgggtcgagttgtccaggttaattagggctgctgctcgagttattagcagtgatataggtacggtgtaataccaagtcagagaagtgcatcgaagaggcaagcttatatgtacgtgacgtgtgattg

SEQ ID NO: 99 - DNA, 708 bp, Zea mays
atggcgcgcgggggagacggcctgcaggtgctgagcgcgctggacgcggcgaagacgcagtggtaccacttcacggccatcatcgtcgccggcatgggcttcttcacggacgcctacgacctcttctgcatctccctcgtcaccaagctgctgggccgcatctactacacggacaccagcaaggacagccccgggtcgctgccgcccaacgtcgcggcggcggtcaacggcgtggccttctgcggcacgctggcggggcagctcttcttcggctggctgggcgacaagctggggcgcaagagcgtgtacgggatgacgctcatggtcatggtcatctgctccgtcgcgtcgggcctctcgttcggccacacccccacgggggtcatggccacgctctgcttcttccgcttctggctcggcttcggcatcggcggcgactacccgctgtcggccaccatcatgtccgagtacgccaacaagaggacccgcgggcgcttcatcgccggcgtcttcgccatgcagggcttcggcatccctccccgggcggcatcgtcacgctcgtcatctcccccgccttccgcgcgggggacccgttcccggcggacaagggacgaacaactttacctccaccgggggcggaaggccgaacactcctggggcggggtcattcagctatggtcttgcggccgcggccggcgcctgcttacc

SEQ ID NO: 100 - DNA, 474 bp, Zea mays
  misc_feature (282)..(282): n = any nucleotide
atgatgaccttcttattgatcggcctccccatcccctaccgaccattgggccacgccgggcaaccaccacgggttcgtcgtcatgtacgccctccccttttcttcgccaacttcgggcccaacagcaccaccttcatcgtgcccgccgagatcttcccggcgcgcgtgcgctccacctgccacggcatctccgccgcctcggggaaggccggggccatcatcggcgcgttcggcttcctgtacgcggcgcagaaccaggacaggagcaagacggacgccggntaccccgcgggcatcggcgtgcgcaactcgctcttcgtcctcgccgccagcaacatgntcggcttcgtccccccgttcctcgtgccggagtccaagggcaagtcgctcgaggagatgtccggtgaggctgaagactcagaggaggagcccgtcggcgcccgtgcggtgcggccgtcggagacccagatggtg

SEQ ID NO: 101 - DNA, 165 bp, Zea mays
  misc_feature (80)..(81): n = any nucleotide
atgggtgtcgggtttgcaagagggctcaatagagtcagggcagagacagttcgcgaaattgtggcgtcctggtgttggtnncacaattccagttggtgctgtcctatctatcttctacacattgatcttaaccaaaattctggcgtacttcatgtgagtggattg

SEQ ID NO: 102 - DNA, 603 bp, Zea mays
atggccgccggggaccttgaggtgctcaccgcgctcgacaccgcgaagacgcaatggtaccacttcaccgccatcgtggtcgccggcatggggttcttcaccgacgcctacgacctcttctgcatctccctcgtgactaagctcctcggccgcatctactacaccgtggaggggtccgcgacgcccggcaccctcccgccgcacgtgtccgcgtccgtcaacggcgtggccttcgtgggcacgctgtcagggcagctcttcttcggctggctgggcgacaagctcgggcgcaagaaggtctatggcatgacgctcatgctcatggtcctctgttccgtcgcgtcggggctctcgttcggccacaccccggcgtccgtcatggcgacgctgtgcttcttccgcttctggctcgggttcggcatcggcggcgactacccgctgtcggcgaccatcatgtccgagtacgccaacaagaagacgcgcggcgcgttcatcgccgcggtgttcgcgatgcagggcttcggcatcatggccggcggcctcgtggccatcgtcgtgtccgcgtcgttcaagggcaggttccccgccccggcctacgccgtc

SEQ ID NO: 103 - DNA, 1719 bp, Oryza sativa
atgtctcaatcttcccctttcttctccgttgcccgagcacatgctggagcaggagggcgagctgcggctgccgctctcctgctccgtcaccctgttgctcagctgccacctcggattcatggcctgaggtactacccttcagccattgtgtcacctgccaagacactgaactcacacctgagccttccccatgccacaatatcctcctttgccaatgctgataatggctccagcgggcaagcagatgccacagagtctgaggaggagcagaacggggaatctgaactgtcggagatggccaaggcattccatatatcgccccggatggcaatgtcaatctcagtggtgattgcatttgcagcgctcactgtgccactggcgatgcagtcagtggtcttccatgggacgaacaaaatgaaggcactggcatatctgacactgctttcagggttctacatggcatggaacattggggcaaatgatgtggccaatgccatggggacgtcagtagggtctggtgctttgacactccggcaagcagtgcttactgcggcggtgctagagttctctggtgcattccttatgggcacccatgtcaccagcaccatgcagaagggcatcctagtcgcatctgtcttccaaggaaaggactctctgctcttcgctggattgttgtcatccctcgctgctgctggaacatggttgcaggttgcttcctcctacggctggcctgtttcaactacacattgtattgtcggggccatggttggttttgggatagtatttggaggcgtaaatgcagtgttctggagctccttggcaagagtatcttcatcatgggtcatttcaccattgatgggtgctgcagtctcatttattgtttacaagggtatacgaaggttcgtatatagtgcaccaaatccaggtcaggctgcagctgctgctgcaccaattgcagtttttactggtgttactgcaatatcatttgccgcttttcctctcagcaagactttttcaatcgctatcctgcaagcattagcatgtggcgcaataggagccgtaatcgttaacagagtgatccaaaaacagcttggtgacctactgtcctcagaagcagaaaagatagcatctgctgataaggcaaatgctcagcaagttggatttctttcggacatagctggaccaacaggagctcaattgcagattgtatatggtgtatttggatacatgcaggtcttgtcagcatgcttcatgtcatttgctcatggaggcaatgatgtctccaatgccataggccccctggcggcagctttgtccattcttcaaggtgtagcaagcagtgctgagatagtcatacctactgaagtccttgcttggggaggttttggaattgttgcagggcttacaatgtggggttacagggtcatagcaacaattggcaagaaaatcaccgaactaacaccaactagaggttttgcagcagagtttgcagcagcttctgtagtattgtttgcatcaaaacttggattgccaatatctgctacacatacacttgttggtgcagtgatgggagtgggatttgcaagagggctcaatagggtcagagcagagacagtgcgtgaaattgtggcctcctggttagtcacaattccagttggtgctgtgttatctatcttctacacattgctcttcacaaagattctggcatactttatgtga

SEQ ID NO: 104 - protein, 572 aa, Oryza sativa
MSQSSPFFSVARAHAGAGGRAAAAALLLRHPVAQLPPRIHGLRYYPSAIVSPAKTLNSHLSLPHATISSFANADNGSSGQADATESEEEQNGESELSEMAKAFHISPRMAMSISVVIAFAALTVPLAMQSVVFHGTNKMKALAYLTLLSGFYMAWNIGANDVANAMGTSVGSGALTLRQAVLTAAVLEFSGAFLMGTHVTSTMQKGILVASVFQGKDSLLFAGLLSSLAAAGTWLQVASSYGWPVSTTHCIVGAMVGFGIVFGGVNAVFWSSLARVSSSWVISPLMGAAVSFIVYKGIRRFVYSAPNPGQAAAAAAPIAVFTGVTAISFAAFPLSKTFSIAILQALACGAIGAVIVNRVIQKQLGDLLSSEAEKIASADKANAQQVGFLSDIAGPTGAQLQIVYGVFGYMQVLSACFMSFAHGGNDVSNAIGPLAAALSILQGVASSAEIVIPTEVLAWGGFGIVAGLTMWGYRVIATIGKKITELTPTRGFAAEFAAASVVLFASKLGLPISATHTLVGAVMGVGFARGLNRVRAETVREIVASWLVTIPVGAVLSIFYTLLFTKILAYFM

SEQ ID NO: 105 - DNA, 1626 bp, Oryza sativa
atggcgcggcaggagcagcagcagcacctgcaggtgctgagcgcgctggacgcggcgaagacgcagtggtaccacttcacggcgatcgtcgtcgccggcatgggcttcttcaccgacgcctacgacctcttctgcatctccctcgtcaccaagctgctcggccgcatctactacaccgacctcgccaaggagaaccccggcagcctgccgcccaacgtcgccgcggcggtgaacggagtcgcgttctgcggcacgctggcggggcagctcttcttcgggtggctcggcgacaagctcggccggaagagcgtgtacgggatgacgctgctgatgatggtcatctgctccatcgcgtcggggctctcgttctcgcacacgcccaccagcgtcatggcgacgctctgcttcttccggttctggctcggattcggcatcggcggcgactacccgctgtcggcgacgatcatgtcggagtacgccaacaagaagacccgcggcgcgttcatcgccgccgtgttcgcgatgcaggggttcggcatcctcgccggcggcatcgtcaccctcatcatctcctccgcgttccgcgccgggttcccggcgccggcgtaccaggacgaccgcgcgggctccaccgtccgccaggccgactacgtgtggcggatcatcctcatgctcggcgccatgccggcgctgctcacctactactggcggatgaagatgccggagacggcgcgctacaccgccctcgtcgccaagaacgccaagcaggccgccgccgacatgtccaaggtgctccaggtcgagatccaggaggagcaggacaagctggagcagatggtgacccggaacagcagcagcttcggcctcttctcccgccagttcgcgcgccgccacggcctccacctcgtcggcaccgccacgacatggttcctcctcgacatcgccttctacagccagaacctgttccagaaggacatcttcaccagcatcaactggatccccaaggccaagaccatgtcggcgctggaggaggtgttccgcatcgcgcgcgcccagacgctcatcgccctgtgcggcaccgtcccgggctactggttcaccgtcttcctcatcgacatcgtcggccgcttcgccatccagctgctagggtttttcatgatgaccgtgttcatgctcggcctcgccgtgccgtaccaccactggacgacgaaggggaaccacatcggcttcgtcgtcatgtacgccttcaccttcttcttcgccaacttcggccccaactccaccaccttcatcgtgccggcggagatcttcccggcgaggctgcgttccacctgccacggcatctcggcggcggcggggaaggccggcgccatcatcggatcgttcgggttcctgtacgcggcgcaggacccgcacaagcccgacgccgggtacaaacccgggatcggggtgaggaactcgctgttcgtgctcgccggatgcaacctgctcgggttcatctgcacgttcctcgtgccggagtcgaaggggaagtcgctggaggagatgtccggcgaggcggaggacgacgacgacgaggtggccgccgccggcggtggcgccgccgtgcggccgcagacggcgtag

SEQ ID NO: 106 - protein, 541 aa, Oryza sativa; identical to SEQ ID NO: 21

SEQ ID NO: 107 - DNA, 1584 bp, Oryza sativa; identical to SEQ ID NO: 1

SEQ ID NO: 108 - protein, 527 aa, Oryza sativa
MAGGQLNVLSTLDQAKTQWYHFMAIVIAGMGFFTDAYDLFCISLVTKLLGRIYYTDDSKDTPGALPPNVSAAVTGVALCGTLAGQLFFGWLGDKLGRKSVYGFTLILMVVCSVASGLSFGSSAKGVVSTLCFFRFWLGFGIGGDYPLSATIMSEYANKRTRGAFIAAVFAMQGFGILFGAIVALAVSAGFRHAYPAPSYSDNHAASLVPQADYVWRIILMFGTVPAALTYYWRMKMPETARYTALIARNAKQAAADMSKVLHTQIEESADRAETVAVGGESWGLFSRQFLRRHGLHLLATTSTWFLLDIAFYSQNLFQKDIFSKVGWIPPAKTMNALEELYRIARAQALIALCGTIPGYWFTVAFIEIMGRFWIQIMGFAMMTAFMLGLAIPYHHWTTPGHHTGFIVMYGFTFFFANFGPNSTTFIVPAEIYPARLRSTCHGISAAAGKAGAIIGAFGFLYAAQDQHKPEPGYPRGIGIKNALFVLAGTNFLGTIMTLLVPESKGMSLEVISQEVADGDDEEAAYPK

(19) United States
(12) Patent Application Publication
(10) Pub. No.: US 2005/0032156 A1
(43) Pub. Date: Feb. 10, 2005

(54) Identification and characterization of phosphate transporter genes

(75) Inventors: Uta Paszkowski, Nenzlingen (CH); Steven P. Briggs, Del Mar, CA (US); Bret Cooper, La Jolla, CA (US); Stephen A. Goff, Research Triangle Park, NC (US); Todd Moughamer, Research Triangle Park, NC (US); Jane Glazebrook, San Diego, CA (US); Fumiaki Katagiri, St.Paul, MN (US); Joel Kreps, Carlsbad, CA (US); Nicolas J. Provart, Toronto (CA); Darrell Ricke, Research Triangle Park, NC (US); Tong Zhu, Research Triangle Park, NC (US)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Participations AG

(21) Appl. No.: 10/481,113

(22) PCT Filed: Jun. 24, 2002
(86) PCT No.: PCT/EP02/06967

Related U.S. Application Data
(60) Provisional application No. 60/300,112, filed on Jun. 22, 2001.
(60) Provisional application No. 60/325,277, filed on Sep. 26, 2001.
(60) Provisional application No. 60/332,064, filed on Nov. 21, 2001.
(60) Provisional application No. 60/361,819, filed on Mar. 4, 2002.

(51) Int. Cl. C12P 21/06

(52) U.S. Cl. 435/69.1

(57) ABSTRACT

The invention relates to proteins that are involved in the transport and translocation of phosphorous in plants and to nucleic acid molecules encoding those proteins. The invention further provides the means and tools for modifying the uptake and translocation of phosphorous in photosynthetic organisms such as plants.

The present invention is in the area of plant biotechnology. In particular, the invention relates to proteins that are involved in the transport and translocation of mineral nutrients such as, for example, phosphorous in plants and to nucleic acid molecules encoding those proteins. The invention further provides the means and tools for modifying the uptake and translocation of phosphorous in photosynthetic organisms such as plants.

Besides nitrogen and potassium, phosphorus is a key element of plant nutrition and the least available of the three nutrients in soil. Phosphorus is essential for plant growth and a structural component of nucleic acids, phospholipids, intermediary metabolites and numerous other biological molecules. As such, phosphorus is involved in photosynthesis, respiration, plant growth and metabolism, and the regulation of many enzymes.

Phosphorus is also part of the adenosine nucleotides, ADP and ATP, which are the currency of cellular energy, and thus critical to bioenergetics. Phosphorylation and dephosphorylation processes, respectively, often function as a kind of regulatory “on/off switch” in cellular metabolism and signal transduction. For example, the phosphorylation and dephosphorylation of certain membrane-bound receptor protein kinases and their substrates are key to various signal transduction pathways, including pathways of plant hormones such as ethylene and abscisic acid. Protein kinases encoded by S-locus genes are also involved in self-incompatibility responses with respect to pollination and fertilization. Regulatory signals can also trigger mechanisms for disease and pest control.

Further, the phosphate to nitrogen ratio in plants is known to affect both the temporal and quantitative characteristics of flowering. Relatively high phosphate advances maturity in plants, whereas relatively low phosphate results in little or no flowering taking place. Phosphate levels are also known to affect the biomass ratio between root and shoot.

In plants, the only readily absorbed form of exogenous phosphorus is inorganic phosphate, the availability of which is limited and varies greatly from place to place. For example, the very acid soils, like that of the tropics, contain many molecules of iron and aluminum, which bind to and tie up nearly all available phosphorus. In alkaline soils, it is the calcium that reacts with the phosphorus and restricts its availability to the plant. When the amount of available phosphate becomes a limiting factor, plant growth and productivity becomes sub-optimal. When phosphate is absent, the plant stops growing and dies. Accordingly, low availability is a major constraint for crop production and phosphorus is considered to be the most limiting nutrient for growth of leguminous crops in tropical and subtropical regions. Surveys indicate that 50% of the leguminous-growing areas in Latin America and Africa are phosphate deficient.

Plants have developed highly specialized mechanisms to overcome low availability of phosphorous in soils, and the biochemical adaptations include the activation of enzymes and an enhanced production of phosphatases and changes in protein phosphorylation. The induction of acid phosphatases in response to phosphorous starvation is a common response in higher plants, as phosphatases liberate Pi from organic materials. These enzymes are considered to be adaptive enzymes of the glycolytic pathway during starvation, and they are involved in “bypass reactions” that circumvent Pi and adenylate-requiring steps in glycolysis. Dicots, especially legumes, are more efficient than monocots in producing and excreting organic acids (malic and citric) to enhance phosphate solubility. Utilizing plants with enhanced malic and citric acid production is of benefit to agricultural areas where phosphate is available but limited.

Enhanced ability for acquisition and altered gene expression are the hallmarks of adaptation to PI deficiency, and the recent discoveries of multiple PI transporters have provided opportunities to elucidate the molecular basis of phosphorous uptake by plants. In Arabidopsis, six high-affinity PI transporter genes have been isolated, but the nature and function of the low affinity PI transporter genes in plants is not clearly understood. Two of these transporter genes, APT1 and APT2 have different promoter sequences but nearly identical coding regions. The latter gene codes for a protein tyrosine phosphatase that is highly sensitive to environmental stress. Under high salt conditions gene expression is enhanced, whereas cold treatment results in a total elimination of transcription.

The use of fertilizers for commercial crops is an expensive, labor intensive, and potentially ecologically damaging practice. Adapting plants to insufficient nutrient conditions diminishes the need for additional fertilizers, and adapting plants to improve their nitrogen, sulfur, and phosphate storage capacity improves plant health and provides a mechanism by which consumer health can be enhanced through the increased nutritional quality of plant food products. Modulating these pathways can also improve plant resistance to disease and pathogens by incorporating enhanced expressions of inhibitory secondary compounds. The potential for ecological remediation may be seen in the effects of plants adapted as environmental scrubbers, whereby such plants can function as accumulators of excessive environmental nitrogen, sulfur and phosphate. Polynucleotides encoding at least one polypeptide involved in these processes, as well as the polypeptides encoded thereby, or any antigene sequences thereof, are commercially useful materials that can be used to study these processes and to modify these processes to elicit desired changes resulting in food products with increased nutritional quality.

Sequence Listing

In the following, a brief description of the sequences in the Sequence Listing is provided:
SEQ ID NOs: 1-7 are nucleotide sequences encoding high-affinity phosphate transporter proteins OsPHT1;1-OsPHT1;7.
SEQ ID NOs: 14-20 provide the amino acid sequence for high-affinity phosphate transporter proteins OsPHT1;1- OsPHT1;7.
SEQ ID NO: 8 and SEQ ID NO: 105 are nucleotide sequences encoding high-affinity phosphate transporter proteins OsPHT1;8.
SEQ ID NO: 21 and SEQ ID NO: 106 provide the amino acid sequence for high-affinity phosphate transporter proteins OsPHT1;8.
SEQ ID NOs: 9-13 are nucleotide sequences encoding high-affinity phosphate transporter proteins OsPHT1;9- OsPHT1;13.
SEQ ID NOs: 22-26 provide the amino acid sequence for high-affinity phosphate transporter proteins OsPHT1;9 -OsPHT1;13.SEQ ID NO: 27 and SEQ ID NO: 103 provides the nucleotide sequences encoding the low-affinity phosphate transporter protein OsPHT2;1.
SEQ ID NO: 28 and SEQ ID NO: 104 provides the amino acid sequences for low-affinity phosphate transporter protein OsPHT2;1.
SEQ ID NOs: 29-41 provide the nucleotide sequence of the promoter region of rice genes encoding high-affinity phosphate transporter proteins OsPHT1;1-OsPHT1;13
SEQ ID NO: 42 provides the nucleotide sequence of the promoter region of a rice gene encoding low-affinity phosphate transporter protein OsPHT2;1
SEQ ID NOs: 43 to 56are OsPHT reverse primers
SEQ ID NO: 57 is a RubQ1 reverse primer
SEQ ID NOs: 58 to 71 are OsPHT forward primers
SEQ ID NO: 72 is a RubQ1 forward primer
SEQ ID NOs: 73 to 87 are OsPHT probe sequences
SEQ ID NOs: 88 to 90 are OsPHT1;11 and OsPHT2;1 promoter forward primers
SEQ ID NOs: 91 to 93 are OsPHT1;11 and OsPHT2;1 promoter reverse primers
SEQ ID NO: 94 is a banana sequence which is homologous to the rice OsPHT sequences
SEQ ID NOs: 95-98 are wheat sequences which are homologous to the rice OsPHT sequences.
SEQ ID NOs: 99-102 are maize sequences which are homologous to the rice OsPHT sequences.
   Definitions

For clarity, certain terms used in the specification are defined and used as follows:

The term “gene” is used broadly to refer to any segment of nucleic acid associated with a biological function. Thus, genes include coding sequences and/or the regulatory sequences required for their expression. For example, gene refers to a nucleic acid fragment that expresses mRNA or functional RNA, or encodes a specific protein, and which includes regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

The term “native” or “wild type” gene refers to a gene that is present in the genome of an untransformed cell, i.e., a cell not having a known mutation.

A “marker gene” encodes a selectable or screenable trait.

The term “chimeric gene” refers to any gene that contains 1) DNA sequences, including regulatory and coding sequences, that are not found together in nature, or 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature.

A “transgene” refers to a gene that has been introduced into the genome by transformation and is stably maintained. Transgenes may include, for example, genes that are either heterologous or homologous to the genes of a particular plant to be transformed. Additionally, transgenes may comprise native genes inserted into a non-native organism, or chimeric genes. The term “endogenous gene” refers to a native gene in its natural location in the genome of an organism. A “foreign” gene refers to a gene not normally found in the host organism but that is introduced by gene transfer.

An “oligonucleotide” corresponding to a nucleotide sequence of the invention, e.g., for use in probing or amplification reactions, may be about 30 or fewer nucleotides in length (e.g., 9, 12, 15, 18, 20, 21 or 24, or any number between 9 and 30). Generally specific primers are upwards of 14 nucleotides in length. For optimum specificity and cost effectiveness, primers of 16 to 24 nucleotides in length may be preferred. Those skilled in the art are well versed in the design of primers for use processes such as PCR. If required, probing can be doneq[with entire restriction fragments of the gene disclosed herein which may be 100's or even 1000's of nucleotides in length.

The terms “protein,” “peptide” and “polypeptide” are used interchangeably herein.

The term “partial-length polypeptide” refers to a fragment obtainable from a full-length polypeptide having substantially the same activity as the full-length polypeptide, e.g., at least 50%, more preferably at least 80%, even more preferably at least 90% to 95% the activity of the full-length polypeptide.

“Coding sequence” refers to a DNA or RNA sequence that codes for a specific amino acid sequence and excludes the non-coding sequences. It may constitute an “uninterrupted coding sequence”, i.e., lacking an intron, such as in a cDNA or it may include one or more introns bounded by appropriate splice junctions. An “intron” is a sequence of RNA which is contained in the primary transcript but which is removed through cleavage and re-ligation of the RNA within the cell to create the mature mRNA that can be translated into a protein.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

A “functional RNA” refers to an antisense RNA, ribozyme, dsRNAi, or other RNA that is not translated.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (MRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences which may be a combination of synthetic and natural sequences. As is noted above, the term “suitable regulatory sequences” is not limited to promoters.

“5′ non-coding sequence” refers to a nucleotide sequence located 5′ (upstream) to the coding sequence. It is present in the fully processed MRNA upstream of the initiation codon and may affect processing of the primary transcript to MnRNA, MRNA stability or translation efficiency (Turner et al., 1995).

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., 1989.

The term “translation leader sequence” refers to that DNA sequence portion of a gene between the promoter and coding sequence that is transcribed into RNA and is present in the fully processed MRNA upstream (5′) of the translation start codon. The translation leader sequence may affect processing of the primary transcript to mRNA, MRNA stability or translation efficiency.

“Signal peptide” refers to the amino terminal extension of a polypeptide, which is translated in conjunction with the polypeptide forming a precursor peptide and which is required for its entrance into the secretory pathway. The term “signal sequence” refers to a nucleotide sequence that encodes the signal peptide.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors which control the effectiveness of transcription initiation in response to physiological or developmental conditions.

The “initiation site” is the position surrounding the first nucleotide that is part of the transcribed sequence, which is also defined as position +1. With respect to this site all other sequences of the gene and its controlling regions are numbered. Downstream sequences (i.e., further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

“Constitutive promoter” refers to a promoter that is able to express the open reading frame (ORF) that it controls in all or nearly all of the plant tissues during all or nearly all developmental stages of the plant. Each of the transcription-activating elements do not exhibit an absolute tissue-specificity, but mediate transcriptional activation in most plant parts at a level of ≧1% of the level reached in the part of the plant in which transcription is most active.

“Regulated promoter” refers to promoters that direct gene expression not constitutively, but in a temporally- and/or spatially-regulated manner, and includes both tissue-specific and inducible promoters. It includes natural and synthetic sequences as well as sequences which may be a combination of synthetic and natural sequences. Different promoters may direct the expression of a gene in different tissues or cell types, or at different stages of development, or in response to different environmental conditions. New promoters of various types useful in plant cells are constantly being discovered, numerous examples may be found in the compilation by Okamuro et al. (1989). Typical regulated promoters useful in plants include but are not limited to safener-inducible promoters, promoters derived from the tetracycline-inducible system, promoters derived from salicylate-inducible systems, promoters derived from alcohol-inducible systems, promoters derived from glucocorticoid-inducible system, promoters derived from pathogen-inducible systems, and promoters derived from ecdysome-inducible systems.

“Tissue-specific promoter” refers to regulated promoters that are not expressed in all plant cells but only in one or more cell types in specific organs (such as leaves or seeds), specific tissues (such as embryo or cotyledon), or specific cell types (such as leaf parenchyma or seed storage cells). These also include promoters that are temporally regulated, such as in early or late embryogenesis, during fruit ripening in developing seeds or fruit, in fully differentiated leaf, or at the onset of senescence.

“Inducible promoter” refers to those regulated promoters that can be turned on in one or more cell types by an external stimulus, such as a chemical, light, hormone, stress, or a pathogen.

“Operably-linked” refers to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation of an endogenous gene, ORF or portion thereof, or a transgene in plants. For example, in the case of antisense constructs, expression may refer to the transcription of the antisense DNA only. In addition, expression refers to the transcription and stable accumulation of sense (MRNA) or functional RNA. Expression may also refer to the production of protein.

“Specific expression” is the expression of gene products which is limited to one or a few plant tissues (spatial limitation) and/or to one or a few plant developmental stages (temporal limitation). It is acknowledged that hardly a true specificity exists: promoters seem to be preferably switch on in some tissues, while in other tissues there can be no or only little activity. This phenomenon is known as leaky expression. However, with specific expression in this invention is meant preferable expression in one or a few plant tissues.

The “expression pattern” of a promoter (with or without enhancer) is the pattern of expression levels which shows where in the plant and in what developmental stage transcription is initiated by said promoter. Expression patterns of a set of promoters are said to be complementary when the expression pattern of one promoter shows little overlap with the expression pattern of the other promoter. The level of expression of a promoter can be determined by measuring the ‘steady state’ concentration of a standard transcribed reporter mRNA. This measurement is indirect since the concentration of the reporter mRNA is dependent not only on its synthesis rate, but also on the rate with which the mRNA is degraded. Therefore, the steady state level is the product of synthesis rates and degradation rates.

The rate of degradation can however be considered to proceed at a fixed rate when the transcribed sequences are identical, and thus this value can serve as a measure of synthesis rates. When promoters are compared in this way techniques available to those skilled in the art are hybridization S1-RNAse analysis, northern blots and competitive RT-PCR. This list of techniques in no way represents all available techniques, but rather describes commonly used procedures used to analyze transcription activity and expression levels of mRNA.

The analysis of transcription start points in practically all promoters has revealed that there is usually no single base at which transcription starts, but rather a more or less clustered set of initiation sites, each of which accounts for some start points of the mRNA. Since this distribution varies from promoter to promoter the sequences of the reporter mRNA in each of the populations would differ from each other. Since each mRNA species is more or less prone to degradation, no single degradation rate can be expected for different reporter mRNAs. It has been shown for various eukaryotic promoter sequences that the sequence surrounding the initiation site (‘initiator’) plays an important role in determining the level of RNA expression directed by that specific promoter. This includes also part of the transcribed sequences. The direct fusion of promoter to reporter sequences would therefore lead to suboptimal levels of transcription.

A commonly used procedure to analyze expression patterns and levels is through determination of the ‘steady state’ level of protein accumulation in a cell. Commonly used candidates for the reporter gene, known to those skilled in the art are β-glucuronidase (GUS), chloramphenicol acetyl transferase (CAT) and proteins with fluorescent properties, such as green fluorescent protein (GFP) from Aequora victoria. In principle, however, many more proteins are suitable for this purpose, provided the protein does not interfere with essential plant functions. For quantification and determination of localization a number of tools are suited. Detection systems can readily be created or are available which are based on, e.g., immunochemical, enzymatic, fluorescent detection and quantification. Protein levels can be determined in plant tissue extracts or in intact tissue using in situ analysis of protein expression.

Generally, individual transformed lines with one chimeric promoter reporter construct will vary in their levels of expression of the reporter gene. Also frequently observed is the phenomenon that such transformants do not express any detectable product (RNA or protein). The variability in expression is commonly ascribed to ‘position effects’, although the molecular mechanisms underlying this inactivity are usually not clear.

“Overexpression” refers to the level of expression in transgenic cells or organisms that exceeds levels of expression in normal or untransformed (nontransgenic) cells or organisms.

“Antisense inhibition” refers to the production of antisense RNA transcripts capable of suppressing the expression of protein from an endogenous gene or a transgene.

“Gene silencing” refers to homology-dependent suppression of viral genes, transgenes, or endogenous nuclear genes. Gene silencing may be transcriptional, when the suppression is due to decreased transcription of the affected genes, or post-transcriptional, when the suppression is due to increased turnover (degradation) of RNA species homologous to the affected genes (English et al., 1996). Gene silencing includes virus-induced gene silencing (Ruiz et al. 1998).

The terms “heterologous DNA sequence,” “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

“Homologous to” in the context of nucleotide sequence identity refers to the similarity between the nucleotide sequence of two nucleic acid molecules or between the amino acid sequences of two protein molecules. Estimates of such homology are provided by either DNA-DNA or DNA-RNA hybridization under conditions of stringency as is well understood by those skilled in the art (as described in Haines and Higgins (eds.), Nucleic Acid Hybridization, IRL Press, Oxford, U.K.), or by the comparison of sequence similarity between two nucleic acids or proteins.

The term “substantially similar” refers to nucleotide and amino acid sequences that represent functional and/or structural equivalents of Oryza sequences disclosed herein.

In its broadest sense, the term “substantially similar” when used herein with respect to a nucleotide sequence means that the nucleotide sequence is part of a gene which encodes a polypeptide having substantially the same structure and function as a polypeptide encoded by a gene for the reference nucleotide sequence, e.g., the nucleotide sequence comprises a promoter from a gene that is the ortholog of the gene corresponding to the reference nucleotide sequence, as well as promoter sequences that are structurally related the promoter sequences particularly exemplified herein, i.e., the substantially similar promoter sequences hybridize to the complement of the promoter sequences exemplified herein under high or very high stringency conditions. For example, altered nucleotide sequences which simply reflect the degeneracy of the genetic code but nonetheless encode amino acid sequences that are identical to a particular amino acid sequence are substantially similar to the particular sequences. The term “substantially similar” also includes nucleotide sequences wherein the sequence has been modified, for example, to optimize expression in particular cells, as well as nucleotide sequences encoding a variant polypeptide having one or more amino acid substitutions relative to the (unmodified) polypeptide encoded by the reference sequence, which substitution(s) does not alter the activity of the variant polypeptide relative to the unmodified polypeptide.

In its broadest sense, the term “substantially similar” when used herein with respect to polypeptide means that the polypeptide has substantially the same structure and function as the reference polypeptide. In addition, amino acid sequences that are substantially similar to a particular sequence are those wherein overall amino acid identity is at least 65% or greater to the instant sequences. Modifications that result in equivalent nucleotide or amino acid sequences are well within the routine skill in the art. The percentage of amino acid sequence identity between the substantially similar and the reference polypeptide is at least 65%, 66%, 67%, 68%, 69%, 70%, e.g., 71%, 72%, 73%, 74%, 75%, 76%, 77%, 78%, 79%, 80%, 81%, 82%, 83%, 84%, 85%, 86%, 87%, 88%, 89%, and even 90% or more, e.g., 91%, 92%, 93%, 94%, 95%, 96%, 97%, 98%, up to at least 99%, wherein the reference polypeptide is an Oryza polypeptide encoded by a gene with a promoter having any one of SEQ ID NOs: 29 to 42, e.g., a nucleotide sequence comprising an open reading frame having any one of SEQ ID NOs: 1 to 13 or 27 which encodes one of SEQ ID NOs: 14 to 26 or 28. One indication that two polypeptides are substantially similar to each other, besides having substantially the same function, is that an agent, e.g., an antibody, which specifically binds to one of the polypeptides, specifically binds to the other.

Sequence comparisons maybe carried out using a Smith-Waterman sequence alignment algorithm (see e.g., Waterman (1995) or http://www hto.usc.edu/software/seqaln/index.html). The localS program, version 1.16, is preferably used with following parameters: match: 1, mismatch penalty: 0.33, open-gap penalty: 2, extended-gap penalty: 2.

Moreover, a nucleotide sequence that is “substantially similar” to a reference nucleotide sequence is said to be “equivalent” to the reference nucleotide sequence. The skilled artisan recognizes that equivalent nucleotide sequences encompassed by this invention can also be defined by their ability to hybridize, under low, moderate and/or stringent conditions (e.g., 0.1×SSC, 0.1% SDS, 65° C.), with the nucleotide sequences that are within the literal scope of the instant statements.

“Target gene” refers to a gene on the replicon that expresses the desired target coding sequence, functional RNA, or protein. The target gene is not essential for replicon replication. Additionally, target genes may comprise native non-viral genes inserted into a non-native organism, or chimeric genes, and will be under the control of suitable regulatory sequences. Thus, the regulatory sequences in the target gene may come from any source, including the virus. Target genes may include coding sequences that are either heterologous or homologous to the genes of a particular plant to be transformed. However, target genes do not include native viral genes. Typical target genes include, but are not limited to genes encoding a structural protein, a seed storage protein, a protein that conveys herbicide resistance, and a protein that conveys insect resistance. Proteins encoded by target genes are known as “foreign proteins”. The expression of a target gene in a plant will typically produce an altered plant trait.

The terms “altered or modified or modulated plant trait” are used interchangeably and mean any phenotypic or genotypic change in a transgenic plant relative to the wild-type or non-transgenic plant host.

“Chromosomally-integrated” refers to the integration of a foreign gene or DNA construct into the host DNA by covalent bonds. Where genes are not “chromosomally integrated” they may be “transiently expressed.” Transient expression of a gene refers to the expression of a gene that is not integrated into the host chromosome but functions independently, either as part of an autonomously replicating plasmid or expression cassette, for example, or as part of another biological system such as a virus.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”. Examples of methods of transformation of plants and plant cells include Agrobacterium-mediated transformation (De Blaere et al., 1987) and particle bombardment technology (Klein et al. 1987; U.S. Pat. No. 4,945,050). Whole plants may be regenerated from transgenic cells by methods well known to the skilled artisan (see, for example, Fromm et al., 1990).

“Transformed,” “transgenic,” and “recombinant” refer to a host organism such as a bacterium or a plant into which a heterologous nucleic acid molecule has been introduced. The nucleic acid molecule can be stably integrated into the genome generally known in the art and are disclosed in Sambrook et al., 1989. See also Innis et al., 1995 and Gelfand, 1995; and Innis and Gelfand, 1999. Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” plants or calli have been through the transformation process and contain a foreign gene integrated into their chromosome. The term “untransformed” refers to normal plants that have not been through the transformation process.

“Transiently transformed” refers to cells in which transgenes and foreign DNA have been introduced (for example, by such methods as Agrobacterium-mediated transformation or biolistic bombardment), but not selected for stable maintenance.

“Stably transformed” refers to cells that have been selected and regenerated on a selection media following transformation.

“Transient expression” refers to expression in cells in which a virus or a transgene is introduced by viral infection or by such methods as Agrobacterium-mediated transformation, electroporation, or biolistic bombardment, but not selected for its stable maintenance.

“Genetically stable” and “heritable” refer to chromosomally-integrated genetic elements that are stably maintained in the plant and stably inherited by progeny through successive generations.

“Primary transformant” and “T0 generation” refer to transgenic plants that are of the same genetic generation as the tissue which is initially transformed (i.e., not having gone through meiosis and fertilization since transformation).

“Secondary transformants” and the “T1, T2, T3, etc. generations” refer to transgenic plants derived from primary transformants through one or more meiotic and fertilization cycles. They may be derived by self-fertilization of primary or secondary transformants or crosses of primary or secondary transformants with other transformed or untransformed plants.

“Wild-type” refers to a virus or organism found in nature without any known mutation.

“Genome” refers to the complete genetic material of an organism.

The term “nucleic acid” or “nucleic acid molecule” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., 1991; Ohtsuka et al., 1985; Rossolini et al. 1994). A “nucleic acid fragment” is a fraction of a given nucleic acid molecule. In higher plants, deoxyribonucleic acid (DNA) is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural or altered nucleotide bases capable of incorporation into DNA or RNA polymers. The terms “nucleic acid” or “nucleic acid molecule” or “nucleic acid sequence” may also be used interchangeably with polynucleotide, gene, cDNA, DNA and RNA encoded by a gene.

The invention encompasses isolated or substantially purified nucleic acid or protein compositions. In the context of the present invention, an “isolated” or “purified” DNA molecule or an “isolated” or “purified” polypeptide is a DNA molecule or polypeptide that, by the hand of man, exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein of interest chemicals.

A “partial-length nucleotide sequence” refers to a part of a full-length nucleotide sequence that encodes a polypeptide having substantially the same activity as the full-length polypeptide, e.g., at least 50%, more preferably at least 80%, even more preferably at least 90% to 95% the activity of the full-length polypeptide

The nucleotide sequences of the invention include both the naturally occurring sequences as well as mutant (variant) forms. Such variants will continue to possess the desired activity, i.e., either promoter activity or the activity of the product encoded by the open reading frame of the non-variant nucleotide sequence.

Thus, by “variants” is intended substantially similar sequences. For nucleotide sequences comprising an open reading frame, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction; (PCR) and hybridization techniques. Variant nucleotide sequences also include synthetically derived nucleotide sequences, such as those generated, for example, by using site-directed mutagenesis and for open reading frames, encode the native protein, as well as those that encode a polypeptide having amino acid substitutions relative to the native protein. Generally, nucleotide sequence variants of the invention will have at least 40, 50, 60, to 70%, e.g., preferably 71%, 72%, 73%, 74%, 75%, 76%,77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98% and 99% nucleotide sequence identity to the native (wild type or endogenous) nucleotide sequence.

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in, each described sequence.

The nucleic acid molecules of the invention can be “optimized” for enhanced expression in plants of interest. See, for example, EPA 035472; WO 91/16432; Perlak et al., 1991; and Murray et al., 1989. In this manner, the open reading frames in genes or gene fragments can be synthesized utilizing plant-preferred codons. See, for example, Campbell and Gowri, 1990 for a discussion of host-preferred codon usage. Thus, the nucleotide sequences can be optimized for expression in any plant. It is recognized that all or any part of the gene sequence may be optimized or synthetic. That is, synthetic or partially optimized sequences may also be used. Variant nucleotide sequences and proteins also encompass sequences and protein derived from a mutagenic and recombinogenic procedure such as DNA shuffling. With such a procedure, one or more different coding sequences can be manipulated to create a new polypeptide possessing the desired properties. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art. See, for example, Stemmer, 1994; Stemmer, 1994; Crameri et al., 1997; Moore et al., 1997; Zhang et al., 1997; Crameri et al., 1998; and U.S. Pat. Nos. 5,605,793 and 5,837,458.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or from human manipulation. Methods for such manipulations are generally known in the art.

Thus, the polypeptides may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. See, for example, Kunkel, 1985; Kunkel et al., 1987; U.S. Pat. No. 4,873,192; Walker and Gaastra, 1983 and the references cited therein. Guidance as to appropriate amino acid substitutions that do not affect biological activity of the protein of interest may be found in the model of Dayhoff et al. (1978). Conservative substitutions, such as exchanging one amino acid with another having similar properties, are preferred.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine I, Lysine (K), Histidine (H); Acidic: Aspartic acid (D), Glutamic acid (E), Asparagine (N), Glutamine (Q). See also, Creighton, 1984. In addition, individual substitutions, deletions or additions which alter, add or delete a single amino acid or a small percentage of amino acids in an encoded sequence are also “conservatively modified variations.”

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one which is naturally occurring but has been obtained in a recombinant form useful for heterologous expression. The expression of the nucleotide sequence in the expression cassette may be under the control of a constitutive promoter or of an inducible promoter which initiates transcription only when the host cell is exposed to some particular external stimulus. In the case of a multicellular organism, the promoter can also be specific to a particular tissue or organ or stage of development.

“Vector” is defined to include, inter alia, any plasmid, cosmid, phage or Agrobacterium binary vector in double or single stranded linear or circular form which may or may not be self transmissible or mobilizable, and which can transform prokaryotic or eukaryotic host either by integration into the cellular genome or exist extrachromosomally (e.g. autonomous replicating plasmid with an origin of replication).

Specifically included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells).

Preferably the nucleic acid in the vector is under the control of, and operably linked to, an appropriate promoter or other regulatory elements for transcription in a host cell such as a microbial, e.g. bacterial, or plant cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell.

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

A “transgenic plant” is a plant having one or more plant cells that contain an expression vector.

“Plant tissue” includes differentiated and undifferentiated tissues or plants, including but not limited to roots, stems, shoots, leaves, pollen, seeds, tumor tissue and various forms of cells and culture such as single cells, protoplast, embryos, and callus tissue. The plant tissue may be in plants or in organ, tissue or cell culture.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm. Preferred, non-limiting examples of such mathematical algorithms are the algorithm of Myers and Miller, 1988; the local homology algorithm of Smith et al. 1981; the homology alignment algorithm of Needleman and Wunsch 1970; the search-for-similarity-method of Pearson and Lipman 1988; the algorithm of Karlin and Altschul, 1990, modified as in Karlin and Altschul, 1993.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. 1988; Higgins et al. 1989; Corpet et al. 1988; Huang et al. 1992; and Pearson et al. 1994. The ALIGN program is based on the algorithm of Myers and Miller, supra. The BLAST programs of Altschul et al., 1990, are based on the algorithm of Karlin and Altschul supra.

Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences (see, e.g., Karlin & Altschul (1993). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

To obtain gapped alignments for comparison purposes, Gapped BLAST (in BLAST 2.0) can be utilized as described in Altschul et al. 1997. Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g. BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=4, and a comparison of both strands. For amino acid sequences, the BLASTP program uses as defaults a wordlength (W) of 3, an expectation (E) of 10, and the BLOSUM62 scoring matrix (see Henikoff & Henikoff, 1989). See http://www.ncbi.nlm.nih.gov. Alignment may also be performed manually by inspection.

For purposes of the present invention, comparison of nucleotide sequences for determination of percent sequence identity to the promoter sequences disclosed herein is preferably made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 71%, 72%, 73%, 74%, 75%, 76%, 77%, 78%, or 79%, preferably at least 80%, 81%, 82%, 83%, 84%, 85%, 86%, 87%, 88%, or 89%, more preferably at least 90%, 91%, 92%, 93%, or 94%, and most preferably at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, more preferably at least 80%, 90%, and most preferably at least 95%.

Another indication that nucleotide sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%, preferably 80%, 81%, 82%, 83%, 84%, 85%, 86%, 87%, 88%, or 89%, more preferably at least 90%, 91%, 92%, 93%, or 94%, or even more preferably, 95%, 96%, 97%, 98% or 99%, sequence identity to the reference sequence over a specified comparison window. Preferably, optimal alignment is conducted using the homology alignment algorithm of Needleman and Wunsch (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridization are sequence dependent, and are different under different environmental parameters. The Tₘ is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl, 1984; Tₘ 81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point I for the specific sequence and its complement at a defined ionic strength and pH. However, severely stringent conditions can utilize a hybridization and/or wash at 1, 2, 3, or 4° C. lower than the thermal melting point I; moderately stringent conditions can utilize a hybridization and/or wash at 6, 7, 8, 9, or 10° C. lower than the thermal melting point I; low stringency conditions can utilize a hybridization and/or wash at 11, 12, 13, 14, 15, or 20° C. lower than the thermal melting point I. Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. An extensive guide to the hybridization of nucleic acids is found in Tijssen, 1993. Generally, highly stringent hybridization and wash conditions are selected to be about 5° C. lower than the thermal melting point Tₘ for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long robes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

The following are examples of sets of hybridization/wash conditions that may be used to clone orthologous nucleotide sequences that are substantially identical to reference nucleotide sequences of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

“DNA shuffling” is a method to introduce mutations or rearrangements, preferably randomly, in a DNA molecule or to generate exchanges of DNA sequences between two or more DNA molecules, preferably randomly. The DNA molecule resulting from DNA shuffling is a shuffled DNA molecule that is a non-naturally occurring DNA molecule derived from at least one template DNA molecule. The shuffled DNA preferably encodes a variant polypeptide modified with respect to the polypeptide encoded by the template DNA, and may have an altered biological activity with respect to the polypeptide encoded by the template DNA.

“Recombinant DNA molecule” is a combination of DNA sequences that are joined together using recombinant DNA technology and procedures used to join together DNA sequences as described, for example, in Sambrook et al., 1989.

The word “plant” refers to any plant, particularly to seed plant, and “plant cell” is a structural and physiological unit of the plant, which comprises a cell wall but may also refer to a protoplast. The plant cell may be in form of an isolated single cell or a cultured cell, or as a part of higher organized unit such as, for example, a plant tissue, or a plant organ.

“Significant increase” is an increase that is larger than the margin of error inherent in the measurement technique, preferably an increase by about 2-fold or greater.

“Significantly less” means that the decrease is larger than the margin of error inherent in the measurement technique, preferably a decrease by about 2-fold or greater.

“High/Low-affinity Phosphate Transporter” The original work of Epstein has identified several distinct classes of regulated transport activities, which are classified into two mechanisms (reviewed in Epstein, 1966, 1972). Mechanism I operates at concentrations below 200 μM and behaves as a saturable carrier. At higher concentrations, mechanism II becomes apparent and displays either linear or multiple saturation kinetics. These mechanisms have also been called high- and low affinity uptake systems, respectively.

“Phosphate deficiency”. Limiting or starvation levels of phosphate result in a “phosphate deficiency” which causes the high-affinity promoter provided by this invention, to activate transcription of a structural gene operably linked to the promoter.

By “deficiency”, it is meant that the level of phosphate uptake is below that required for the immediate requirements of a plant or photosynthetic organism.

The term “starvation” refers to a level of phosphate available to the plant which is not only limiting, but is below that required for normal maintenance and/or growth wherein, if phosphate is maintained at that level, the plant or photosynthetic organism would eventually die for lack of adequate phosphorus.

The present invention relates to proteins that are involved in the transport and translocation of phosphorous in plants and to nucleic acid molecules encoding those proteins. The invention further provides the means and tools for modifying the uptake and translocation of phosphorous in photosynthetic organisms such as plants.

Specific embodiments of the present invention relate to

an isolated nucleic acid molecule comprising a nucleotide sequence encoding a polypeptide that mediates phosphate uptake into the plant cell in the presence of a mycorrhizal fungus such as, for example, an arbuscular mycorrhizal fungus (AMF), but not under conditions of phosphate deficiency or phosphate sufficiency,and, optionally, a regulatory region, which induces a mycorrhiza dependent and phosphate independent transcription of the coding region.
an isolated nucleic acid molecule comprising a nucleotide sequence encoding a polypeptide that mediates phosphate uptake into the plant cell in the presence of a mycorrhizal fungus such as, for example, an arbuscular mycorrhizal fungus, but not under conditions of phosphate deficiency or phosphate sufficiency, and, optionally, a regulatory region, which induces a mycorrhiza dependent and phosphate independent transcription of the coding region, which nucleic acid molecule is substantially similar to a nucleic acid encoding a polypeptide as given in SEQ ID NO: 24, or a partial-length polypeptide having substantially the same activity as the full-length polypeptide, e.g., at least 50%, more preferably at least 80%, even more preferably at least 90% to 95% the activity of the full-length polypeptide.
an isolated nucleic acid molecule comprising a nucleotide sequence encoding a polypeptide that mediates phosphate uptake into the plant cell in the presence of a mycorrhizal fungus such as, for example, an arbuscular mycorrhizal fungus, but not under conditions of phosphate deficiency or phosphate sufficiency, and, optionally, a regulatory region, which induces a mycorrhiza dependent and phosphate independent transcription of the coding region which polypeptide is substantially similar, and preferably has at least between 70%, and 99% amino acid sequence identity to the polypeptide of SEQ ID NO: 24, with any individual number within this range of between 70% and 99% also being part of the invention, or a partial-length polypeptide having substantially the same activity as the full-length polypeptide, e.g., at least 50%, more preferably at least 80%, even more preferably at least 90% to 95% the activity of the full-length polypeptide.
an isolated nucleic acid molecule comprising a nucleotide sequence encoding a polypeptide that mediates phosphate uptake into the plant cell in the presence of a mycorrhizal fungus such as, for example, an arbuscular mycorrhizal fungus, but not under conditions of phosphate deficiency or phosphate sufficiency, and, optionally, a regulatory region, which induces a mycorrhiza dependent and phosphate independent transcription of the coding region, which polypeptide is immunologically reactive with antibodies raised against a polypeptide of SEQ ID NO: 24, or a partial-length polypeptide having substantially the same activity as the full-length polypeptide, e.g., at least 50%, more preferably at least 80%, even more preferably at least 90% to 95% the activity of the full-length polypeptide.
an isolated nucleic acid molecule comprising a nucleotide sequence
   a) as given in SEQ ID NO: 11; or a part thereof, which still encodes a partial-length polypeptide having substantially the same activity as the full-length polypeptide, e.g., at least 50%, more preferably at least 80%, even more preferably at least 90% to 95% the activity of the full-length polypeptide;
   b) having substantial similarity to (a);
   c) capable of hybridizing to (a) or the complement thereof;
   d) capable of hybridizing to a nucleic acid comprising 50 to 200 or more consecutive nucleotides of a nucleotide sequence given in SEQ ID NO: 11 or the complement thereof;
   e) complementary to (a), (b) or (c); ; or
   f) which is the reverse complement of (a), (b) or (c).
an isolated nucleic acid molecule comprising a plant nucleotide sequence that induces transcription of a linked nucleic acid segment in a plant or plant cell, e.g., a linked nucleic acid molecule comprising an open reading frame for or encoding a structural or regulatory gene, in the presence of a mycorrhizal fungus such as, for example, an arbuscular mycorrhizal fungus, but not under conditions of phosphate deficiency or phosphate sufficiency.
an isolated nucleic acid molecule comprising a plant nucleotide sequence that induces transcription of a linked nucleic acid segment in a plant or plant cell and is preferably obtained or obtainable from plant genomic DNA comprising a gene having an open reading frame (ORF) encoding a polypeptide which is substantially similar, and preferably has at least between 70%, and 99% amino acid sequence identity, to a polypeptide encoded by an Oryza, e.g., Oryza sativa, gene comprising a nucleotide sequence as given in SEQ ID NO: 11 wherein said nucleotide sequence induces transcription of a linked nucleic acid segment in response to the presence of a mycorrhizal fungus such as, for example, an arbuscular mycorrhizal fungus, but not under conditions of phosphate deficiency or phosphate sufficiency.
an isolated nucleic acid molecule comprising a plant nucleotide sequence which is the promoter sequence for a gene comprising an ORF that is substantially similar, and preferably has at least between 70% to 99% nucleic acid sequence identity, to an Oryza gene comprising the sequence as given in SEQ ID NO: 11 and a fragment thereof which has substantially the same promoter activity as the corresponding promoter listed in SEQ ID NO: 39.
an isolated nucleic acid molecule comprising a plant nucleotide sequence which induces transcription of a linked nucleic acid segment in a plant or plant cell, e.g., a linked plant DNA comprising an open reading frame for a structural or regulatory gene, in response to the presence of a mycorrhizal fungus such as, for example, an arbuscular mycorrhizal fungus, but not under conditions of phosphate deficiency or phosphate sufficiency, and comprises a nucleotide sequence
   (a) as given in SEQ ID NO: 39; or a partial-length nucleotide sequence having substantially the same activity as the full-length nucleotide sequence, e.g., at least 50%, more preferably at least 80%, even more preferably at least 90% to 95% the activity of the full-length nucleotide sequence;
   (b) having substantial similarity to (a);
   (c) capable of hybridizing to (a)) or the complement thereof; or
   (d) capable of hybridizing to a nucleic acid comprising 50 to 200 or more consecutive nucleotides of a nucleotide sequence given in SEQ ID NO: 39 or the complement thereof;
   (e) complementary to (a), (b) or (c); or
   (f) which is the reverse complement of (a), (b) or (c).
An isolated nucleic acid molecule comprising a nucleotide sequence encoding a polypeptide that mediates phosphate uptake into the plant cell in the presence of a mycorrhizal fungus such as, for example, an arbuscular mycorrhizal fungus, but not under conditions of phosphate deficiency or phosphate sufficiency, which polypeptide is substantially similar to a polypeptide encoded by a nucleotide sequence comprising a promoter sequence as given in SEQ ID NO: 39, and, optionally, a regulatory region, which induces such a mycorrhiza dependent and phosphate independent transcription of the coding region.
An isolated nucleic acid molecule comprising a nucleotide sequence encoding a polypeptide that mediates phosphate uptake into the plant cell in the presence of a mycorrhizal fungus such as, for example, an arbuscular mycorrhizal fungus, but not under conditions of phosphate deficiency or phosphate sufficiency, which is nucleotide sequence is substantially similar to a nucleotide sequence comprising a promoter sequence as given in SEQ ID NO: 39, and, optionally, a regulatory region, which induces such a mycorrhiza dependent and phosphate independent transcription of the coding region.
An isolated nucleic acid molecule comprising a nucleotide sequence encoding a polypeptide that mediates phosphate uptake into the plant cell in the presence of a mycorrhizal fungus such as, for example, an arbuscular mycorrhizal fungus, but not under conditions of phosphate deficiency or phosphate sufficiency, wherein the regulatory region comprises a promoter sequence as given in SEQ ID NO: 39, and, optionally, a regulatory region, which induces such a mycorrhiza dependent and phosphate independent transcription of the coding region.
In a further embodiment of the invention, the nucleic acid molecule according to the invention as mentioned hereinbefore comprises a nucleotide sequence encoding a polypeptide that mediates phosphate uptake into the plant cell in the presence of a mycorrhizal fungus such as, for example, an arbuscular mycorrhizal fungus, but not under conditions of phosphate deficiency or phosphate sufficiency, which polypeptide is preferentially expressed in plant roots, and, optionally, a regulatory region, which induces a mycorrhiza dependent and phosphate independent transcription of the coding region.
   Another embodiment of the invention relates to
an isolated nucleic acid molecule comprising a nucleotide sequence encoding a polypeptide that mediates phosphate uptake into the plant cell in response to phosphate availability, that is under conditions of
   (a) phosphate deficiency, or;
   (b) phosphate sufficiency
perferentially in a specific plant tissue, i.e., in roots, green tissue- (leaf- and stem-) or flowers, and, optionally, a regulatory region, which induces such a phosphate dependent transcription of the coding region.
an isolated nucleic acid molecule comprising a nucleotide sequence encoding a polypeptide that mediates phosphate uptake into the plant cell in response to phosphate availability, which polypeptide is preferentially expressed in a specific plant tissue, i.e., in roots, green tissue- (leaf- and stem-) or flowers, and, optionally, a regulatory region, which induces a phosphate dependent transcription of the coding region.
an isolated nucleic acid molecule comprising a nucleotide sequence encoding a polypeptide that mediates phosphate uptake into the plant cell in response to phosphate availability, which nucleic acid molecule is substantially similar, and preferably has at least between 70% and 99% amino acid sequence identity, to a nucleic acid encoding a polypeptide as given in SEQ ID NO: 14 to 23 and 25 to 26 and 28 and 104, with each individual number within this range of between 70% and 99% also being part of the invention, or a partial-length polypeptide having substantially the same activity as the full-length polypeptide, e.g., at least 50%, more preferably at least 80%, even more preferably at least 90% to 95% the activity of the full-length polypeptide; and, optionally, a regulatory region, which induces such a phosphate-dependent transcription of the coding region.
an isolated nucleic acid molecule comprising a nucleotide sequence encoding a polypeptide that mediates phosphate uptake into the plant cell in response to phosphate availability, which polypeptide is substantially similar, and preferably has at least between 70% and 99% amino acid sequence identity, to a polypeptide encoded by an Oryza gene as given in any one of the sequences selected from the group consisting of SEQ ID NOs: 1 to 10 and 12 to 13, 27, 103 and 105, a partial-length polypeptide having substantially the same activity as the full-length polypeptide, e.g., at least 50%, more preferably at least 80%, even more preferably at least 90% to 95% the activity of the full-length polypeptide, and is preferentially expressed in a specific plant tissue, i.e., in roots, green tissue- (leaf- and stem-tissue) or flowers; and, optionally, a regulatory region, which induces such a phosphate-dependent transcription of the coding region.
an isolated nucleic acid molecule comprising a nucleotide sequence encoding a polypeptide that mediates phosphate uptake into the plant cell in response to phosphate availability, which polypeptide specifically binds to an antibody that has been generated against a polypeptide of SEQ ID NO: 14 to 23 and 25 to 26 and 28 and 104, or a partial-length polypeptide having substantially the same activity as the full-length polypeptide, e.g., at least 50%, more preferably at least 80%, even more preferably at least 90% to 95% the activity of the full-length polypeptide, and is preferentially expressed in a specific plant tissue, i.e., in roots, green tissue- (leaf- and stem-) or flowers; and, optionally, a regulatory region, which induces such a phosphate-dependent transcription of the coding region.
an isolated nucleic acid molecule comprising a nucleotide sequence
   (a) as given in SEQ ID NO: 1 to 10, 12 to 13, 27, 103 and 105; or a part thereof still encoding a partial-length polypeptide having substantially the same activity as the full-length polypeptide, e.g., at least 50%, more preferably at least 80%, even more preferably at least 90% to 95% the activity of the full-length polypeptide
   (b) having substantial similarity to (a);
   (c) capable of hybridizing to (a) or the complement thereof;
   (d) capable of hybridizing to a nucleic acid comprising 50 to 200 or more consecutive nucleotides of a nucleotide sequence given in SEQ ID NO: 1 to 10, 12 to 13, 27, 103 and 105 or the complement thereof;
   (e) complementary to (a), (b) or (c); or
   (f) which is the reverse complement of (a), (b) or (c).
an isolated nucleic acid molecule comprising a plant nucleotide sequence that induces transcription of a linked nucleic acid segment in a plant or plant cell, e.g., a linked nucleic acid molecule comprising an open reading frame for or encoding a structural or regulatory gene, in response to phosphate availability, that is under conditions of
   (a) phosphate deficiency, or;
   (b) phosphate sufficiency.
an isolated nucleic acid molecule comprising a plant nucleotide sequence that induces transcription of a linked nucleic acid segment in a plant or plant cell and is preferably obtained or obtainable from plant genomic DNA comprising a gene having an open reading frame (ORF) encoding a polypeptide which is substantially similar, and preferably has at least between 70%, and 99% amino acid sequence identity, to a polypeptide encoded by an Oryza, e.g., Oryza sativa, gene comprising a nucleotide sequence as given in SEQ ID NOs: 1 to 10, 12 to 13, 27, 103 and 105, with each individual number falling within this range of between 70% to 99% also being part of the invention, wherein said nucleotide sequence induces transcription of a linked nucleic acid segment in response to phosphate availability, that is under conditions of (a) phosphate deficiency, or; (b) phosphate sufficiency.
an isolated nucleic acid molecule comprising a nucleotide sequence which is the promoter sequence for a gene comprising an ORF that is substantially similar, and preferably has at least between 70% to 99% nucleic acid sequence identity, to an Oryza gene comprising the sequence as given in SEQ ID NO: 1 to 10, 12 to 13, 27, 103 and 105, with each individual number falling within this range of between 70% to 99% also being part of the invention, and a fragment thereof which has substantially the same promoter activity as the corresponding promoter listed in SEQ ID NO: 29 to 38 and 40 to 42.
an isolated nucleic acid molecule comprising a nucleotide sequence which induces transcription of a linked nucleic acid segment in a plant or plant cell, e.g., a linked plant DNA comprising an open reading frame for a structural or regulatory gene, in response to phosphate availability, that is under conditions of (a) phosphate deficiency, or (b) phosphate sufficiency, and comprises a nucleotide sequence
   (a) as given in SEQ ID NO: 29 to 38 and 40 to 42; or a partial-length nucleotide sequence having substantially the same activity as the full-length nucleotide sequence, e.g., at least 50%, more preferably at least 80%, even more preferably at least 90% to 95% the activity of the full-length nucleotide sequence;
   (b) having substantial similarity to (a);
   (c) capable of hybridizing to (a) or the complement thereof;
   (d) capable of hybridizing to a nucleic acid comprising 50 to 200 or more consecutive nucleotides of a nucleotide sequence given in SEQ ID NO: 29 to 38 and 40 to 42 or the complement thereof;
   (e) complementary to (a), (b) or (c); or
   (f) which is the reverse complement of (a), (b) or (c).
An isolated nucleic acid molecule comprising a nucleotide sequence encoding a polypeptide that mediates phosphate uptake into the plant cell in response to phosphate availability, that is under conditions of (a) phosphate deficiency, or (b) phosphate sufficiency, which polypeptide is substantially similar to a polypeptide encoded by a nucleotide sequence comprising a promoter sequence as given in SEQ ID NO: 29 to 38 and 40 to 42, and, optionally, a regulatory region, which induces such a phosphate-dependent transcription of the coding region.
An isolated nucleic acid molecule comprising a nucleotide sequence encoding a polypeptide that mediates phosphate uptake into the plant cell in response to phosphate availability, that is under conditions of (a) phosphate deficiency, or (b) phosphate sufficiency, which nucleic acid molecule is substantially similar to a nucleotide sequence comprising a promoter sequence as given in SEQ ID NO: 29 to 38 and 40 to 42; and, optionally, a regulatory region, which induces such a phosphate-dependent transcription of the coding region.
An isolated nucleic acid molecule comprising a nucleotide sequence encoding a polypeptide that mediates phosphate uptake into the plant cell and a regulatory region, which induces transcription of the coding region in the presence of phosphate availability, that is under conditions of (a) phosphate deficiency, or (b) phosphate sufficiency, wherein the regulatory region comprises a promoter sequence as given in SEQ ID NO: 29 to 38 and 40 to 42.
In a further embodiment, the isolated nucleic acid molecule of the invention as mentioned hereinbefore comprising a nucleotide sequence encoding a polypeptide that mediates phosphate uptake into the plant cell under conditions of phosphate deficiency, is preferentially expressed in plant root, flower, and green tissue- (leaf- and stem-) tissue, respectively.
In another embodiment, the isolated nucleic acid molecule of the invention as mentioned hereinbefore comprising a nucleotide sequence encoding a polypeptide that mediates phosphate uptake into the plant cell under conditions of phosphate sufficiency, is preferentially expressed in plant green tissue- (leaf- and stem-) tissue.
The invention further relates to high and low affinity transporter proteins which are encoded by the nucleic acid molecules according to the invention. In particular, the invention relates to phosphate transporter proteins which are substantially similar to those provided in SEQ ID NOs: 14 to 26, or to a partial-length polypeptide having substantially the same activity as the full-length polypeptide, e.g., at least 50%, more preferably at least 80%, even more preferably at least 90% to 95% the activity of the full-length polypeptide, which are of the high affinity type and are preferentially expressed in specific plant tissues, i.e., plant root, flower, and shoot tissue.
In a specific specific embodiment the invention relates to a phosphate transporter protein as given in SEQ ID NO: 24, or a partial-length polypeptide having substantially the same activity as the full-length polypeptide, e.g., at least 50%, more preferably at least 80%, even more preferably at least 90% to 95% the activity of the full-length polypeptide, which is of the high-affinity type and highly transcribed during symbiotic interaction with a mycorrhizal fungus such as, for example, an arbuscular mycorrhizal fungus.
In another specific embodiment the invention relates to a phosphate transporter protein as given in SEQ ID NO: 15, or a partial-length polypeptide having substantially the same activity as the full-length polypeptide, e.g., at least 50%, more preferably at least 80%, even more preferably at least 90% to 95% the activity of the full-length polypeptide, which is of the high-affinity type and expressed preferentially in roots and upregulated by phosphate starvation, but is downregulated by mycorrhizal fungi.
In a further specific embodiment, the invention relates to a phosphate transporter protein as given in SEQ ID NO: 20, or a partial-length polypeptide having substantially the same activity as the full-length polypeptide, e.g., at least 50%, more preferably at least 80%, even more preferably at least 90% to 95% the activity of the full-length polypeptide, which is of the high-affinity type and expressed preferentially in flowers.
In still another specific embodiment, the invention relates to a phosphate transporter protein as given in SEQ ID NO: 28, or a partial-length polypeptide having substantially the same activity as the full-length polypeptide, e.g., at least 50%, more preferably at least 80%, even more preferably at least 90% to 95% the activity of the full-length polypeptide, which is of the low-affinity type and expressed preferentially in green tissue- (leaf- and stem-).
   Further encompassed within the invention is
a recombinant vector containing a chimeric polynucleotide according to embodiments of the present invention. In particular, the recombinant vector according to the invention contains an isolated nucleic acid molecule comprising a plant nucleotide sequence that induces transcription of a linked nucleic acid segment in a plant or plant cell in the presence of a mycorrhizal fungus such as, for example, an arbuscular mycorrhizal fungus, but not under conditions of phosphate deficiency or phosphate sufficiency, operably linked to either its native coding sequence encoding a phosphate transporter protein of the high-affinity type or a heterologous coding sequence or a regulatory sequence. In a further embodiment of the invention the isolated nucleic acid molecule induces transcription of any linked nucleic acid segment preferentially in root tissue.
Further encompassed is a recombinant vector that contains an isolated nucleic acid molecule comprising a plant nucleotide sequence that induces transcription of a linked nucleic acid segment in a plant or plant cell in response to phosphate availability, that is under conditions of phosphate sufficiency, operably linked to either its native coding sequence encoding a phosphate transporter protein of the low-affinity type or a heterologous coding sequence or a regulatory sequence. In a further embodiment of the invention the isolated nucleic acid molecule induces transcription of any linked nucleic acid segment preferentially in root or green tissue- (leaf- and stem-) tissue or both.
Further encompassed is a recombinant vector that contains an isolated nucleic acid molecule comprising a plant nucleotide sequence that induces transcription of a linked nucleic acid segment in a plant or plant cell in response to phosphate availability, that is under conditions of (a) phosphate deficiency, operably linked to either its native coding sequence encoding a phosphate transporter protein of the high-affinity type or a heterologous coding sequence or a regulatory sequence. In a further embodiment of the invention the isolated nucleic acid molecule induces transcription of any linked nucleic acid segment preferentially in root, stem or flower tissue or a combination of two or all of them.
Also encompassed are plant cells, which contain chimeric polynucleotides, according to the present disclosure, and plants, containing these plant cells. In a preferred embodiment, the plant is a dicot. In another preferred embodiment, the plant is a gymnosperm. In another preferred embodiment, the plant is a monocot. In a more preferred embodiment, the monocot is a cereal. In a more preferred embodiment, the cereal may be, for example, maize, wheat, barley, oats, rye, millet, sorghum, triticale, secale, einkorn, spelt, emmer, teff, milo, flax, gramma grass, Tripsacum and teosinte. In a most preferred embodiment, the cereal is rice.
In one embodiment, the chimeric polynucleotide is expressed in a specific location or tissue of a plant. In a preferred embodiment, the location or tissue may be, for example, epidermis, root, vascular tissue, meristem, cambium, cortex, pith, green tissue, preferably leaf or stem, and flower, preferably panicle and pollen. In a more preferred embodiment, the location or tissue is a seed, preferably embryo, aleurone or endosperm.
   Further embodiments of the invention provide a
method of modulating phosphate-uptake into in a photosynthetic organism, but especially in a plant, comprising:
   a) inserting an isolated nucleic acid molecule according to the invention comprising a nucleic acid fragment encoding a phosphate transporter protein according to the invention under the transcriptional control of a symbiotically regulated promoter sequence into a cell, group of cells, tissue or organ of a photosynthetic organism;
   b) maintaining the cell, group of cells, tissue or organ in an association with a symbiontic fungus such as, for example, an arbuscular mycorrhizal fungus (AMF) so that transcription of the phosphate transporter encoding DNA sequence is induced and the phosphate transporter protein is expressed.
In a specific embodiment phosphate uptake is modulated in the plant root by using a promoter fragment according to the invention that in addition induces tissue preferential expression of the associated coding sequence in roots.
method of modulating phosphate-uptake into in a photosynthetic organism, but especially in a plant, comprising:
   a) inserting an isolated nucleic acid molecule according to the invention comprising a nucleic acid fragment encoding a phosphate transporter protein according to the invention under the transcriptional control of a phosphate regulated promoter sequence into a cell, group of cells, tissue or organ of a photosynthetic organism;
   b) maintaining the cell, group of cells, tissue or organ under conditions of phosphate sufficiency or phosphate deficiency so that transcription of the phosphate transporter encoding DNA sequence is induced and the phosphate transporter protein is expressed.
In a specific embodiment phosphate uptake is modulated in the plant root, green tissue (leaf or stem), flower (pollen) or a combination of two or more of said tissues, by using a phosphate-regulated promoter fragment according to the invention that in addition induces tissue preferential expression of the associated coding sequence in roots, green tissue (leaf or stem), flowers (pollen) or two or more of said tissues.

The present invention provides the ability to regulate genes of interest, including phosphor transporter genes, either in response to a mycorrhizal fungus such as, for example, an arbuscular mycorrhizal fungus (AMF), upon symbinontically interacting with the plant root system or under conditions of phosphate sufficiency and deficiency, respectively. In a further embodiment of the invention, a tissue specific component is added to the regulation in that promoter regions are provided herein that are not only responsive to symbiontic interactions with mycorrhizal fungi and phosphate levels in the plants surrounding, but, in addition, are capable of initiating transcription of any operably linked gene such as a coding or regulatory DNA sequence in a tissue preferential or tissue specific manner.

Phosphorus is one of the essential but scarcely available plant macronutrients and approximately 80% of the artificially applied phosphorus becomes unavailable to crops as it is rapidly adsorbed, precipitated, converted into the organic form or washed away into ground water. Arbuscular mycorrhizal (AM) symbiosis improves phosphate availability to plants (reviewed in Smith and Read, 1997). This benefit is based on the considerable increase of the soil volume to be explored for nutrient assimilation by forming far-reaching extraradical mycelia operating as functional extensions of the plant root system. In consequence mineral nutrition, in particular phosphate nutrition, of the plant hosts is enhanced thereby improving plant growth in natural environments but also crop productivity especially on less intensively fertilized soils.

In modern agricultural environments intensive phosphate fertilization, necessary for best possible yield, limits benefits of the AM symbiosis. However, considering the devastating effects of intensive artificial fertilization on aquatic systems it could be envisage that the agriculture of the future could benefit from exploration of the symbiotic nutrient exchange in order to decrease negative environmental impacts.

AM fungi associate intimately with the roots of more than 80% of terrestrial plants by growing inter- and intracellularly in the cortex of their host roots. Highly differentiated haustoria, so called arbuscules, are formed inside individual cortex cells and due to the concomitant drastic enlargement of the plant plasma membrane a huge surface area for contact and nutrient exchange is created (reviewed in Harrison, 1999).

Inorganic phosphate (Pi) is taken up by roots as orthophosphate, concentrations of which rarely exceed 10 μM in the soil water. Due to the slow diffusion of Pi within the soil and its immediate uptake a Pi depletion zone is rapidly created around roots (reviewed in Schachtman et al., 1998). Plants have acquired a number of different strategies to maximize Pi uptake under such Pi limiting conditions. Similar to yeast plants explore high and low affinity phosphate transporter systems. While low affinity phosphate transporter are constitutively expressed and operate at Pi concentrations in the millimolar range, genes for high affinity phosphate transporter are only transcriptionally induced and contribute to phosphate uptake at limiting, micromolar phosphate concentrations (reviewed in Schachtman et al., 1998, Chrispeels et al., 1999, Raghothama, 2000). An additional set of Pi transporters participates in the translocation of Pi throughout the plant. Such functional specialization is reflected by the complexity of the Pi transporter gene family. Over the past years a number of high affinity Pi transporter genes have been cloned from different plant species via the identification of expressed sequence tags with sequence homologies to yeast Pi transporters (Bun-ya et al., 1991, Martinez and Persson, 1998) and by further using these as heterologous probes to screen plant root cDNA libraries (Raghothama, 2000). As a result one low affinity phosphate transporter has been cloned from Arabidopsis thaliana and several high affinity transporters have been reportedly identified from various dicotyledonous plants, namely Arabidopsis thaliana, Solanum tuberosum, Lycopersicon esculentum and Medicago truncatula (reviewed by Raghothama, 2000). The accessibility of the whole genome information in Arabidopsis thaliana now allows for the determination of its entire collection of Pi transporter. Nine putative high affinity transporters of Arabidopsis are computationally identified but since Arabidopsis belongs to the small minority of plants that do not associate with the AM fungi, only Pi uptake independent on mycorrhizae can be studied. The genome of Oryza sativa cv. Nipponbare has recently been sequenced, and as shown herein rice very efficiently uses AM symbiosis. Rice therefore supplies an opportunity to obtain the full picture of Pi acquisition activities including those operating in the absence of mycorrhizae, plus those operating in the presence of mycorrhizae.

For the known Pi transporter it has been reported that while the one low affinity transporter is preferentially and constitutively expressed in green tissue, most of the high affinity transporter transcripts accumulated in roots and are induced upon Pi deprivation. The majority of the high affinity transporter genes are additionally expressed in other types of tissue and hence, each of these genes could be potentially involved in phosphate uptake at the root-soil interface but may also have additional roles in phosphate translocation within the plant (reviewed in Raghothama, 2000).

Importantly, an additional plant encoded uptake systems appears to be involved in the acquisition of Pi delivered into the cortex of plant roots by AM fungi. The proposed route of the fungus-mediated Pi uptake involves the initial uptake of Pi from the soil by fungal hyphae and transport to the highly differentiated intracellular arbuscules, where it is supposed to be released into the interface between fungus and plant cortex cells. Subsequently Pi is acquired from the interface by a plant Pi uptake system (reviewed in Harrison, 1999).

Previously the transcript of one tomato Pi transporter (LePT1) is reported to accumulate in mycorrhizal roots. In situ hybridization showed that the LePT1 messenger is moderately expressed in arbusculated cortex cells. The same gene had formerly been shown to be activated in epidermis, root cap, root hairs, vascular system and in cells of the palisade parenchyma of Pi deprived plants and therefore it seems to participate rather generally in Pi uptake and translocation.

Within the scope of the present invention, a genome wide search for the full set of phosphate transporter genes in rice has been employed in search of a potential candidate Pi transporter working specifically at the plant fungus interface,. As shown in the Examples, among the 13 genes encoding homologs of high affinity phosphate transporters as taught herein (refered to as Oryza sativa Phosphate Transporter (OsPHT) genes), a transporter gene designated OsPHT1;11 responded with a very interesting specific and strong induction of expression to mycorrhizal colonization. OsPHT1;11 expression could not be detected in any plant organ at any given developmental stage in the absence of AM fungi. However, root colonization by AM fungi led to strong transcriptional induction of the OsPHT1;11 gene. This induction is confined to the root system and is tightly correlated with the degree of root colonization by Glomus intraradices. OsPHT1;11 activation is neither dependent on the nutritional status of the plant nor on the phosphate availability in the rhizosphere since supplementary phosphate had no inductive effect on the OsPHT1;11 gene consistent with a regulation specific to the presence of the symbiotic fungus. Moreover, infection of roots with the fungal pathogen Rhizoctonia solani did not activate OsPHT1;11 further corroborating the high specificity of OsPHT1;11 for the AM symbiosis. OsPHT1;11 expression complemented the mutation of the yeast high affinity phosphate transporter mutant (pho84) and thereby confirming its function. OsPHT1;11 is shown to be a mycorrhizae specific plant phosphate transporter, thus uncovering an astonishing functional partition of nutrient uptake and exchange systems operating between plants and AM fungi.

In one embodiment, the invention provides a promoter region that is obtainable from plant genomic DNA, but especially from genomic DNA of rice plants, such as that given in SEQ ID NO: 39, which combines root preferential transcription of operably linked DNA molecules with the capability to specifically induce transcription in response to symbiontic interactions with mycorrhizal fungi.

In another embodiment, the invention provides a promoter region that is obtainable from plant genomic DNA, but especially from genomic DNA of rice plants, such as that given in SEQ ID NO: 30, which combines root preferential transcription of operably linked DNA molecules with the capability to specifically induce transcription of associated DNA molecules under conditions of phosphate deficiency. These promoter regions are further capable of downregulating transcription of associated DNA molecules in the presence of mycorrhizal fungi.

In still another embodiment, the invention provides a promoter region that is obtainable from plant genomic DNA, but especially from genomic DNA of rice plants, such as that given in SEQ ID NO: 35 which combines flower preferential transcription of operably linked DNA molecules with the capability to specifically induce transcription of associated DNA molecules under conditions of phosphate deficiency.

In still another embodiment, the invention provides a promoter region that is obtainable from plant genomic DNA, but especially from genomic DNA of rice plants, such as that given in SEQ ID NO: 42, which combines green tissue- (leaf- and stem-) preferential transcription of operably linked DNA molecules with the capability to specifically induce transcription of associated DNA molecules under conditions of phosphate sufficiency.

The promoters of the invention include a consecutive stretch of about 25 to 2000, including 50 to 500 or 100 to 250, and up to 1000 or 1500, contiguous nucleotides, e.g., 40 to about 750, 60 to about 750, 125 to about 750, 250 to about 750, 400 to about 750, 600 to about 750, of any one of SEQ ID NOs: 29 to 42, or the promoter orthologs thereof, which include the minimal promoter region.

In a particular embodiment of the invention said consecutive stretch of about 25 to 2000, including 50 to 500 or 100 to 250, and up to 1000 or 1500, contiguous nucleotides, e.g., 40 to about 750, 60 to about 750, 125 to about 750, 250 to about 750, 400 to about 750, 600 to about 750, has at least 75%, preferably 80%, more preferably 90% and most preferably 95%, nucleic acid sequence identity with a corresponding consecutive stretch of about 25 to 2000, including 50 to 500 or 100 to 250, and up to 1000 or 1500, contiguous nucleotides, e.g., 40 to about 750, 60 to about 750, 125 to about 750, 250 to about 750, 400 to about 750, 600 to about 750, of any one of SEQ ID NOs: 29 to 42 or the promoter orthologs thereof, which include the minimal promoter region. The above defined stretch of contiguous nucleotides preferably comprises one or more promoter motifs selected from the group consisting of TATA box, GC-box, CAAT-box and a transcription start site.

In case of promoters directing tissue-specific transcription of a linked nucleic acid segment in a plant or plant cell such as, for example, a promoter directing root-specific, green tissue- (leaf- and stem-)-specific, flower-specific, etc., transcription, it is further preferred that previously defined stretch of contiguous nucleotides comprises further motifs that participate in the tissue specificity of said stretch(es) of nucleotides.

Generally, the promoters of the invention may be employed to express a nucleic acid segment that is operably linked to said promoter such as, for example, an open reading frame, or a portion thereof, an anti-sense sequence, or a transgene in plants. The open reading frame may be obtained from an insect resistance gene, a disease resistance gene such as, for example, a bacterial disease resistance gene, a fungal disease resistance gene, a viral disease resistance gene, a nematode disease resistance gene, a herbicide resistance gene, a gene affecting grain composition or quality, a nutrient utilization gene, a mycotoxin reduction gene, a male sterility gene, a selectable marker gene, a screenable marker gene, a negative selectable marker, a positive selectable marker, a gene affecting plant agronomic characteristics, i.e., yield, standability, and the like, or an environment or stress resistance gene, i.e., one or more genes that confer herbicide resistance or tolerance, insect resistance or tolerance, disease resistance or tolerance (viral, bacterial, fungal, oomycete, or nematode), stress tolerance or resistance (as exemplified by resistance or tolerance to drought, heat, chilling, freezing, excessive moisture, salt stress, or oxidative stress), increased yields, food content and makeup, physical appearance, male sterility, drydown, standability, prolificacy, starch properties or quantity, oil quantity and quality, amino acid or protein composition, and the like. By “resistant” is meant a plant which exhibits substantially no phenotypic changes as a consequence of agent administration, infection with a pathogen, or exposure to stress. By “tolerant” is meant a plant which, although it may exhibit some phenotypic changes as a consequence of infection, does not have a substantially decreased reproductive capacity or substantially altered metabolism.

For instance, flower-specific promoters, but especially pollen-specific promoters such as that given in SEQ ID NO: 35, may be employed to introduce genes into pollen for the purpose of arresting pollen development thereby rendering a plant male sterile. Such genes may include those coding for proteins toxic to pollen. It is also contemplated that chimeric plasmids may be constructed which allow the expression of antisense mRNAs which are capable of inhibiting expression of genes which play a role in pollen development. It is also contemplated that expression cassettes or vectors of the present invention which comprise a pollen-specific promoter may be useful for the introduction of one or more useful phenotypic characteristics into pollen including but not limited to pesticide resistance, resistance to insect pests or toxicity to insect pests, or which optimize other pollen functions. One embodiment the invention comprises genetic manipulation of plants to potentiate the effects of gibberellin or other hormones involved in initiation of fruit set. The invention comprises the temporal expression of a structural gene which encodes a plant hormone such as a gibberellin or cytokine, or proteins associated with the production of such hormones (i.e,. enzymes, biosynthetic intermediates and the like.) which are associated with initiation of fruit set. The structural gene is placed under the control of a pollen microspore- or megaspore-specific promoter such that the expression of the hormone is timed to occur just prior to pollination so that fruit development and maturation is induced without the need for fertilization.

Root-specific promoters such as those given in SEQ ID NOs: 30 and 39 may be useful for expressing genes including but not limited to defense-related genes, including genes conferring insecticidal resistance and stress tolerance, e.g., salt, cold or drought tolerance, genes for altering nutrient uptake and genes that are involved with specific morphological traits that allow for increased water absorption, uptake or extraction from soil, e.g., soil of low moisture content. For example, introduction and expression of genes that alter root characteristics may enhance water uptake. Additionally, the use of root-specific promoters in transgenic plants can provide beneficial traits that are localized in the consumable (by animals and humans) roots of plants such as carrots, parsnips, and beets. However, other parts of the plants, including stalks, husks, vegetative parts, and the like, may also be desirable, including use as part of animal silage or for ornamental purposes. Often chemical constituents (e.g., oils or starches) of maize and other crops are extracted for foods or industrial use and transgenic plants may be created which have enhanced or modified levels of such components.

Green tissue- (leaf- and stem-)-specific promoters such as that given in SEQ ID NO: 42 may be useful for expressing genes including but not limited to genes involved in photosynthetic pathways, and for those which are green tissue- (leaf- and stem-)-specific, for producing large quantities of protein, and for expressing oils or proteins of interest, genes for increasing the nutritional value of a plant, and defense-related genes (e.g., against pathogens such as a virus or fungus), including genes encoding insecticidal polypeptides.

Obtaining sufficient levels of transgene expression in the appropriate plant tissues is an important aspect in the production of genetically engineered crops. Expression of heterologous DNA sequences in a plant host is dependent upon the presence of an operably linked promoter that is functional within the plant host. Choice of the promoter sequence will determine when and where within the organism the heterologous DNA sequence is expressed.

It is specifically contemplated by the present invention that one could use any one of the promoters according to the present invention in unaltered or altered form. Mutagenization of a promoter of the present invention such as those provided in SEQ ID NOs: 29 to 42 may potentially improve the utility of the elements for the expression of transgenes in plants. The mutagenesis of these elements can be carried out at random and the mutagenized promoter sequences screened for activity in a trial-by-error procedure.

Alternatively, particular sequences which provide the promoter with desirable expression characteristics, or the promoter with expression enhancement activity, could be identified and these or similar sequences introduced into the sequences via mutation. It is further contemplated that one could mutagenize these sequences in order to enhance their expression of transgenes in a particular species.

The means for mutagenizing a DNA segment encoding a promoter sequence of the current invention are well-known to those of skill in the art. As indicated, modifications to promoter or other regulatory element may be made by random, or site-specific mutagenesis procedures. The promoter and other regulatory element may be modified by altering their structure through the addition or deletion of one or more nucleotides from the sequence which encodes the corresponding unmodified sequences.

Mutagenesis may be performed in accordance with any of the techniques known in the art, such as, and not limited to, synthesizing an oligonucleotide having one or more mutations within the sequence of a particular regulatory region. In particular, site-specific mutagenesis is a technique useful in the preparation of promoter mutants, through specific mutagenesis of the underlying DNA. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to about 75 nucleotides or more in length is preferred, with about 10 to about 25 or more residues on both sides of the junction of the sequence being altered.

In general, the technique of site-specific mutagenesis is well known in the art, as exemplified by various publications. As will be appreciated, the technique typically employs a phage vector which exists in both a single stranded and double stranded form. Typical vectors useful in site-directed mutagenesis include vectors such as the M13 phage. These phage are readily commercially available and their use is generally well known to those skilled in the art.

Double stranded plasmids also are routinely employed in site directed mutagenesis which eliminates the step of transferring the gene of interest from a plasmid to a phage.

In general, site-directed mutagenesis in accordance herewith is performed by first obtaining a single-stranded vector or melting apart of two strands of a double stranded vector which includes within its sequence a DNA sequence which encodes the promoter. An oligonucleotide primer bearing the desired mutated sequence is prepared, generally synthetically. This primer is then annealed with the single-stranded vector, and subjected to DNA polymerizing enzymes such as E. coli polymerase I KYenow fragment, in order to complete the synthesis of the mutation-bearing strand. Thus, a heteroduplex is formed wherein one strand encodes the original non-mutated sequence and the second strand bears the desired mutation.

This heteroduplex vector is then used to transform or transfect appropriate cells, such as E. coli cells, and cells are selected which include recombinant vectors bearing the mutated sequence arrangement. Vector DNA can then be isolated from these cells and used for plant transformation. A genetic selection scheme is devised by Kunkel et al. (1987) to enrich for clones incorporating mutagenic oligonucleotides. Alternatively, the use of PCR with commercially available thermostable enzymes such as Taq polymerase may be used to incorporate a mutagenic oligonucleotide primer into an amplified DNA fragment that can then be cloned into an appropriate cloning or expression vector. The PCR-mediated mutagenesis procedures of Tomic et al. (1990) and Upender et al. (1995) provide two examples of such protocols. A PCR employing a thermostable ligase in addition to a thermostable polymerase also may be used to incorporate a phosphorylated mutagenic oligonucleotide into an amplified DNA fragment that may then be cloned into an appropriate cloning or expression vector. The mutagenesis procedure described by Michael (1994) provides an example of one such protocol.

The preparation of sequence variants of the selected promoter-encoding DNA segments using site-directed mutagenesis is provided as a means of producing potentially useful species and is not meant to be limiting as there are other ways in which sequence variants of DNA sequences may be obtained. For example, recombinant vectors encoding the desired promoter sequence may be treated with mutagenic agents, such as hydroxylamine, to obtain sequence variants.

As used herein, the term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” also is intended to refer to a process that involves the template-dependent extension of a primer molecule. The term template-dependent process refers to nucleic acid synthesis of an RNA or a DNA molecule wherein the sequence of the newly synthesized strand of nucleic acid is dictated by the well-known rules of complementary base pairing (see, for example, Watson and Rarnstad, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224. A number of template dependent processes are available to amplify the target sequences of interest present in a sample, such methods being well known in the art and specifically disclosed herein below.

Where a clone comprising a promoter has been isolated in accordance with the instant invention, one may wish to delimit the essential promoter regions within the clone. One efficient, targeted means for preparing mutagenizing promoters relies upon the identification of putative regulatory elements within the promoter sequence. This can be initiated by comparison with promoter sequences known to be expressed in similar tissue-specific or developmentally unique manner. Sequences which are shared among promoters with similar expression patterns are likely candidates for the binding of transcription factors and are thus likely elements which confer expression patterns. Confirmation of these putative regulatory elements can be achieved by deletion analysis of each putative regulatory region followed by functional analysis of each deletion construct by assay of a reporter gene which is functionally attached to each construct. As such, once a starting promoter sequence is provided, any of a number of different deletion -mutants of the starting promoter could be readily prepared.

As indicated above, deletion mutants, deletion mutants of the promoter of the invention also could be randomly prepared and then assayed. With this strategy, a series of constructs are prepared, each containing a different portion of the clone (a subclone), and these constructs are then screened for activity. A suitable means for screening for activity is to attach a deleted promoter or intron construct which contains a deleted segment to a selectable or screenable marker, and to isolate only those cells expressing the marker gene. In this way, a number of different, deleted promoter constructs are identified which still retain the desired, or even enhanced, activity. The smallest segment which is required for activity is thereby identified through comparison of the selected constructs. This segment may then be used for the construction of vectors for the expression of exogenous genes.

Furthermore, it is contemplated that promoters combining elements from more than one promoter may be useful. For example, U.S. Pat. No. 5,491,288 discloses combining a Cauliflower Mosaic Virus promoter with a histone promoter. Thus, the elements from the promoters disclosed herein may be combined with elements from other promoters

According to one embodiment, the present invention is directed to a nucleic acid molecule comprising a nucleotide sequence isolated or obtained from any plant which encodes a polypeptide having at least 70% amino acid sequence identity to a polypeptide encoded by a gene comprising any one of SEQ ID NOs:1 to 13, 27, 103 and 105.

Based on the Oryza nucleic acid sequences of the present invention as given in SEQ ID NOs: 1 to 13, 27, and 29-42, orthologs may be identified or isolated from the genome of any desired organism, preferably from another plant, according to well known techniques based on their sequence similarity to the Oryza nucleic acid sequences, e.g., hybridization, PCR or computer generated sequence comparisons. For example, all or a portion of a particular Oryza nucleic acid sequence is used as a probe that selectively hybridizes to other gene sequences present in a population of cloned genomic DNA fragments or cDNA fragments (i.e., genomic or cDNA libraries) from a chosen source organism. Further, suitable genomic and cDNA libraries may be prepared from any cell or tissue of an organism. Such techniques include hybridization screening of plated DNA libraries (either plaques or colonies; see, e.g., Sambrook et al., 1989) and amplification by PCR using oligonucleotide primers preferably corresponding to sequence domains conserved among related polypeptide or subsequences of the nucleotide sequences provided herein (see, e.g., Innis et al., 1990). These methods are particularly well suited to the isolation of gene sequences from organisms closely related to the organism from which the probe sequence is derived. The application of these methods using the Oryza sequences as probes is well suited for the isolation of gene sequences from any source organism, preferably other plant species. In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art.

In hybridization techniques, all or part of a known nucleotide sequence is used as a probe that selectively hybridizes to other corresponding nucleotide sequences present in a population of cloned genomic DNA fragments or cDNA fragments (i.e., genomnic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the sequence of the invention. Methods for preparation of probes for hybridization and for construction of cDNA and genomic libraries are generally known in the art and are disclosed in Sambrook et al. (1989). In general, sequences that hybridize to the sequences disclosed herein will have at least 40% to 50%, about 60% to 70% and even about 80% 85%, 90%, 95% to 98% or more identity with the disclosed sequences. That is, the sequence similarity of sequences may range, sharing at least about 40% to 50%, about 60% to 70%, and even about 80%, 85%, 90%, 95% to 98% sequence similarity, with each individual number within the ranges given above also being part of the invention.

The nucleic acid molecules of the invention can also be identified by, for example, a search of known databases for genes encoding polypeptides having a specified amino acid sequence identity or DNA having a specified nucleotide sequence identity. Methods of alignment of sequences for comparison are well known in the art and are described hereinabove.

The present invention further provides a composition, an expression cassette or a recombinant vector containing the nucleic acid molecule of the invention, and host cells comprising the expression cassette or vector, e.g., comprising a plasmid. In particular, the present invention provides an expression cassette or a recombinant vector comprising a promoter of the invention such as, for example, those given in SEQ ID NOs: 29 to 42 linked to a nucleic acid segment which, when present in a plant, plant cell or plant tissue, results in transcription of the linked nucleic acid segment. The invention also provides an expression cassette or a recombinant vector comprising a plant nucleotide sequence comprising an open reading frame of the invention such as, for example, those given in SEQ ID NOs: 1 to 13, 27, 103 and 105 which, when present in a plant, plant cell or plant tissue, results in expression of the protein product encoded by the open reading frame. Further, the invention provides isolated polypeptides encoded by any one of the open reading frames comprising SEQ ID NOs: 1 to 13, 27, 103 and 105, a fragment thereof which encodes a polypeptide which has substantially the same activity as the corresponding polypeptide encoded by an ORF listed in SEQ ID NOs: 1 to 13, 27, 103 and 105, or the orthologs thereof.

Virtually any DNA composition may be used for delivery to recipient plant cells, e.g., monocotyledonous cells, to ultimately produce fertile transgenic plants in accordance with the present invention. For example, DNA segments or fragments in the form of vectors and plasmids, or linear DNA segments or fragments, in some instances containing only the DNA element to be expressed in the plant, and the like, may be employed. The construction of vectors which may be employed in conjunction with the present invention will be known to those of skill of the art in light of the present disclosure (see, e.g., Sambrook et al., 1989; Gelvin et al., 1990).

It is one of the objects of the present invention to provide recombinant DNA molecules comprising a nucleotide sequence which directs transcription according to the invention operably linked to a nucleic acid segment or sequence of interest.

In one specific embodiment, the invention provides an expression cassette or vector containing an isolated nucleic acid molecule having a nucleotide sequence that directs tissue-specific, tissue-preferential and/or mycorrhizal fungi- or phosphate-induced transcription of a linked nucleic acid segment of interest in a cell, which nucleotide sequence is from a gene which encodes a polypeptide having at least 70% identity to an Oryza polypeptide encoded by a gene having one of the promoters listed in SEQ ID NOs: 29 to 42, and which nucleotide sequence is optionally operably linked to other suitable regulatory sequences, e.g., a transcription terminator sequence, operator, repressor binding site, transcription factor binding site and/or an enhancer.

Furthermore, the nucleic acid segment of interest can, for example, code for a ribosomal RNA, an antisense RNA or any other type of RNA that is not translated into protein. In another preferred embodiment of the invention, the nucleic acid segment of interest is translated into a protein product. The nucleotide sequence which directs transcription and/or the nucleic acid segment may be of homologous or heterologous origin with respect to the plant to be transformed. A recombinant DNA molecule useful for introduction into plant cells includes that which has been derived or isolated from any source, that may be subsequently characterized as to structure, size and/or function, chemically altered, and later introduced into plants. An example of a nucleotide sequence or segment of interest “derived” from a source, would be a nucleotide sequence or segment that is identified as a useful fragment within a given organism, and which is then chemically synthesized in essentially pure form. An example of such a nucleotide sequence or segment of interest “isolated” from a source, would be nucleotide sequence or segment that is excised or removed from said source by chemical means, e.g., by the use of restriction endonucleases, so that it can be further manipulated, e.g., amplified, for use in the invention, by the methodology of genetic engineering. Such a nucleotide sequence or segment is commonly referred to as “recombinant.”

Therefore a useful nucleotide sequence, segment or fragment of interest includes completely synthetic DNA, semi-synthetic DNA, DNA isolated from biological sources, and DNA derived from introduced RNA. Generally, the introduced DNA is not originally resident in the plant genotype which is the recipient of the DNA, but it is within the scope of the invention to isolate a gene from a given plant genotype, and to subsequently introduce multiple copies of the gene into the same genotype, e.g., to enhance production of a given gene product such as a storage protein or a protein that confers tolerance or resistance to water deficit or a protein of the invention as provided in SEQ D NOs: 14 to 26, 28, 104 and 106.

The introduced recombinant DNA molecule includes but is not limited to, DNA from plant genes, and non-plant genes such as those from bacteria, yeasts, animals or viruses. The introduced DNA can include modified genes, portions of genes, or chimeric genes, including genes from the same or different genotype.

The introduced recombinant DNA molecule used for transformation herein may be circular or linear, double-stranded or single-stranded. Generally, the DNA is in the form of chimeric DNA, such as plasmid DNA, that can also contain coding regions flanked by regulatory sequences which promote the expression of the recombinant DNA present in the resultant plant.

Generally, the introduced recombinant DNA molecule will be relatively small, i.e., less than about 30 kb to minimize any susceptibility to physical, chemical, or enzymatic degradation which is known to increase as the size of the nucleotide molecule increases. As noted above, the number of proteins, RNA transcripts or mixtures thereof which is introduced into the plant genome is preferably preselected and defined, e.g., from one to about 5-10 such products of the introduced DNA may be formed.

This expression cassette or vector may be contained in a host cell. The expression cassette or vector may augment the genome of a transformed plant or may be maintained extrachromosomally. The expression cassette may be operatively linked to a structural gene, the open reading frame thereof, or a portion thereof. The expression cassette may further comprise a Ti plasmid and be contained in an Agrobacterium tumefaciens cell; it may be carried on a microparticle, wherein the microparticle is suitable for ballistic transformation of a plant cell; or it may be contained in a plant cell or protoplast. Further, the expression cassette or vector can be contained in a transformed plant or cells thereof, and the plant may be a dicot or a monocot. In particular, the plant may be a cereal plant.

Two principal methods for the control of expression are known, viz.: overexpression and underexpression. Overexpression can be achieved by insertion of one or more than one extra copy of the selected gene. It is, however, not unknown for plants or their progeny, originally transformed with one or more than one extra copy of a nucleotide sequence, to exhibit the effects of underexpression as well as overexpression. For underexpression there are two principle methods which are commonly referred to in the art as “antisense downregulation” and “sense downregulation” (sense downregulation is also referred to as “cosuppression”). Generically these processes are referred to as “gene silencing”. Both of these methods lead to an inhibition of expression of the target gene.

The invention hence also provides sense and anti-sense nucleic acid molecules corresponding to the open reading frames identified in SEQ ID NOs: 1 to 13, 27, 103 and 105 as well as their orthologs.

The genes and open reading frames according to the present invention which are substantially similar to a nucleotide sequence encoding a polypeptide as given in SEQ ID NOs: 14 to 26, 28, 104 and 106 including any corresponding anti-sense constructs can be operably linked to any promoter that is functional within the plant host including the promoter sequences according to the invention or mutants thereof.

Further promoters which are useful for plant transgene expression include those that are inducible, viral, synthetic, constitutive (Odell et al., 1985), temporally regulated, spatially regulated, tissue-specific, and spatio-temporally regulated.

Where expression in specific tissues or organs is desired, tissue-specific promoters may be used. In contrast, where gene expression in response to a stimulus is desired, inducible promoters are the regulatory elements of choice. Where continuous expression is desired throughout the cells of a plant, constitutive promoters are utilized. Additional regulatory sequences upstream and/or downstream from the core promoter sequence may be included in expression constructs of transformation vectors to bring about varying levels of expression of heterologous nucleotide sequences in a transgenic plant.

A variety of 5N and 3N transcriptional regulatory sequences are available for use in the present invention. Transcriptional terminators are responsible for the termination of transcription and correct mRNA polyadenylation. The 3N nontranslated regulatory DNA sequence preferably includes from about 50 to about 1,000, more preferably about 100 to about 1,000, nucleotide base pairs and contains plant transcriptional and translational termination sequences. Appropriate transcriptional terminators and those which are known to function in plants include the CaMV 35S terminator, the tml terminator, the nopaline synthase terminator, the pea rbcS E9 terminator, the terminator for the T7 transcript from the octopine synthase gene of Agrobacterium tumefaciens, and the 3N end of the protease inhibitor I or II genes from potato or tomato, although other 3N elements known to those of skill in the art can also be employed. Alternatively, one also could use a gamma coixin, oleosin 3 or other terminator from the genus Coix.

Preferred 3N elements include those from the nopaline synthase gene of Agrobacterium tumefaciens (Bevan et al., 1983), the terminator for the T7 transcript from the octopine synthase gene of Agrobacterium tumefaciens, and the 3′ end of the protease inhibitor I or II genes from potato or tomato.

As the DNA sequence between the transcription initiation site and the start of the coding sequence, i.e., the untranslated leader sequence, can influence gene expression, one may also wish to employ a particular leader sequence. Preferred leader sequences are contemplated to include those which include sequences predicted to direct optimum expression of the attached gene, i.e., to include a preferred consensus leader sequence which may increase or maintain mRNA stability and prevent inappropriate initiation of translation. The choice of such sequences will be known to those of skill in the art in light of the present disclosure. Sequences that are derived from genes that are highly expressed in plants will be most preferred.

Other sequences that have been found to enhance gene expression in transgenic plants include intron sequences (e.g., from Adh1, bronze1, actin1, actin 2 (WO 00/760067), or the sucrose synthase intron) and viral leader sequences (e.g., from TMV, MCMV and AMV). For example, a number of non-translated leader sequences derived from viruses are known to enhance expression. Specifically, leader sequences from Tobacco Mosaic Virus (TMV), Maize Chlorotic Mottle Virus (MCMV), and Alfalfa Mosaic Virus (AMV) have been shown to be effective in enhancing expression (e.g., Gallie et al., 1987; Skuzeski et al., 1990). Other leaders known in the art include but are not limited to: Picomavirus leaders, for example, EMCV leader (Encephalomyocarditis 5 noncoding region) (Elroy-Stein et al., 1989); Potyvirus leaders, for example, TEV leader (Tobacco Etch Virus); MDMV leader (Maize Dwarf Mosaic Virus); Human immunoglobulin heavy-chain binding protein (BiP) leader, (Macejak et al., 1991); Untranslated leader from the coat protein MRNA of alfalfa mosaic virus (AMV RNA 4), (Jobling et al., 1987; Tobacco mosaic virus leader (TMv), (Gallie et al., 1989; and Maize Chlorotic Mottle Virus leader (MCMV) (Lommel et al., 1991. See also, Della-Cioppa et al., 1987.

Regulatory elements such as Adh intron 1 (Callis et al., 1987), sucrose synthase intron (Vasil et al., 1989) or TMV omega element (Gallie, et al., 1989), may further be included where desired.

Examples of enhancers include elements from the CaMV 35S promoter, octopine synthase genes (Ellis el al., 1987), the rice actin I gene, the maize alcohol dehydrogenase gene (Callis et al., 1987), the maize shrunken I gene (Vasil et al., 1989), TMV Omega element (Gallie et al., 1989) and promoters from non-plant eukaryotes (e.g. yeast; Ma et al., 1988).

The present invention further provides a method of augmenting a plant genome by contacting plant cells with a nucleic acid molecule of the invention, e.g., one having a nucleotide sequence that directs tissue-specific, tissue-preferential and/or mycorrhizal fungi- or phosphate-induced transcription of a linked nucleic acid segment isolatable or obtained from a plant gene encoding a polypeptide that is substantially similar to a polypeptide encoded by the an Oryza gene having a sequence according to any one of SEQ ID NOs: 1 to 13, 27, 103 and 105 so as to yield transformed plant cells; and regenerating the transformed plant cells to provide a differentiated transformed plant, wherein the differentiated transformed plant expresses the nucleic acid molecule in the cells of the plant. The nucleic acid molecule may be present in the nucleus, chloroplast, mitochondria and/or plastid of the cells of the plant.

Plant species may be transformed with the DNA construct of the present invention by the DNA-mediated transformation of plant cell protoplasts and subsequent regeneration of the plant from the transformed protoplasts in accordance with procedures well known in the art.

Any plant tissue capable of subsequent clonal propagation, whether by organogenesis or embryogenesis, may be transformed with a vector of the present invention. The term “organogenesis,” as used herein, means a process by which shoots and roots are developed sequentially from meristematic centers; the term “embryogenesis,” as used herein, means a process by which shoots and roots develop together in a concerted fashion (not sequentially), whether from somatic cells or gametes. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristems, axillary buds, and root meristems), and induced meristem tissue (e.g., cotyledon meristem and ultilane meristem).

Plants of the present invention may take a variety of forms. The plants may be chimeras of transformed cells and non-transformed cells; the plants may be clonal transformants (e.g., all cells transformed to contain the expression cassette); the plants may comprise grafts of transformed and untransformed tissues (e.g., a transformed root stock grafted to an untransformed scion in citrus species). The transformed plants may be propagated by a variety of means, such as by clonal propagation or classical breeding techniques. For example, first generation (or T1) transformed plants may be selfed to give homozygous second generation (or T2) transformed plants, and the T2 plants further propagated through classical breeding techniques. A dominant selectable marker (such as npt II) can be associated with the expression cassette to assist in breeding.

Thus, the present invention provides a transformed (transgenic) plant cell, in planta or ex planta, including a transformed plastid or other organelle, e.g., nucleus, mitochondria or chloroplast. The present invention may be used for transformation of any plant species, including, but not limited to, cells from corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticumn aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea ultilane), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, duckweed (Lemna), barley, vegetables, ornamentals, and conifers.

Duckweed (Lemna, see WO 00/07210) includes members of the family Lemnaceae.; There are known four genera and 34 species of duckweed as follows: genus Lemna (L. aequinoctialis, L. disperma, L. ecuadoriensis, L. gibba, L. japonica, L. minor, L. miniscula, L. obscura, L. perpusilla, L. tenera, L. trisulca, L. turionifera, L. valdiviana); genus Spirodela (S. intermedia, S. polyrrhiza, S. punctata); genus Woffia (Wa. Angusta, Wa. Arrhiza, Wa. Australina, Wa. Borealis, Wa. Brasiliensis, Wa. Columbiana, Wa. Elongata, Wa. Globosa, Wa. Microscopica, Wa. Neglecta) and genus Wofiella (W1. ultila, W1. ultilanen, W1. gladiata, W1. ultila, W1. lintgulata, W1. repunda, W1. rotunda, and W1. neotropica). Any other genera or species of Lemnaceae, if they exist, are also aspects of the present invention. Lemna gibba, Lemna minor, and Lemna miniscula are preferred, with Lemna minor and Lemna miniscula being most preferred. Lemna species can be classified using the taxonomic scheme described by Landolt, Biosystematic Investigation on the Family of Duckweeds: The family of Lemnaceae—A Monograph Study. Geobatanischen Institut ETH, Stiftung Rubel, Zurich (1986)).

Vegetables within the scope of the invention include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata), Douglas-fir (Pseudotsuga menziesii); Western hemlock (Tsuga ultilane); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Leguminous plants include beans and peas. Beans include guar, locust bean, fenugreek, soybean, garden beans, cowpea, mungbean, lima bean, fava bean, lentils, chickpea, etc. Legumes include, but are not limited to, Arachis, e.g., peanuts, Vicia, e.g., crown vetch, hairy vetch, adzuki bean, mung bean, and chickpea, Lupinus, e.g., lupine, trifolium, Phaseolus, e.g., common bean and lima bean, Pisum, e.g., field bean, Melilotus, e.g., clover, Medicago, e.g., alfalfa, Lotus, e.g., trefoil, lens, e.g., lentil, and false indigo. Preferred forage and turf grass for use in the methods of the invention include alfalfa, orchard grass, tall fescue, perennial ryegrass, creeping bent grass, and redtop.

Other plants within the scope of the invention include Acacia, aneth, artichoke, arugula, blackberry, canola, cilantro, clementines, escarole, eucalyptus, fennel, grapefruit, honey dew, jicama, kiwifruit, lemon, lime, mushroom, nut, okra, orange, parsley, persimmon, plantain, pomegranate, poplar, radiata pine, radicchio, Southern pine, sweetgum, tangerine, triticale, vine, yams, apple, pear, quince, cherry, apricot, melon, hemp, buckwheat, grape, raspberry, chenopodium, blueberry, nectarine, peach, plum, strawberry, watermelon, eggplant, pepper, cauliflower, Brassica, e.g., broccoli, cabbage, ultilan sprouts, onion, carrot, leek, beet, broad bean, celery, radish, pumpkin, endive, gourd, garlic, snapbean, spinach, squash, turnip, ultilane, and zucchini.

Ornamental plants within the scope of the invention include impatiens, Begonia, Pelargonium, Viola, Cyclamen, Verbena, Vinca, Tagetes, Primula, Saint Paulia, Agertum, Amaranthus, Antihirrhinum, Aquilegia, Cineraria, Clover, Cosmo, Cowpea, Dahlia, Datura, Delphinium, Gerbera, Gladiolus, Gloxinia, Hippeastrum, Mesembryanthemum, Salpiglossos, and Zinnia. Other plants within the scope of the invention are shown in Table 1 (above).

Preferably, transgenic plants of the present invention are crop plants and in particular cereals (for example, corn, alfalfa, sunflower, rice, Brassica, canola, soybean, barley, soybean, sugarbeet, cotton, safflower, peanut, sorghum, wheat, millet, tobacco, etc.), and even more preferably corn, rice and soybean.

The present invention also provides a transgenic plant prepared by this method, a seed from such a plant and progeny plants from such a plant including hybrids and inbreds. Preferred transgenic plants are transgenic maize, soybean, barley, alfalfa, sunflower, canola, soybean, cotton, peanut, sorghum, tobacco, sugarbeet, rice, wheat, rye, turfgrass, millet, sugarcane, tomato, or potato.

A transformed (transgenic) plant of the invention includes plants, the genome of which is augmented by a nucleic acid molecule of the invention, or in which the corresponding gene has been disrupted, e.g., to result in a loss, a decrease or an alteration, in the function of the product encoded by the gene, which plant may also have increased yields and/or produce a better-quality product than the corresponding wild-type plant. The nucleic acid molecules of the invention are thus useful for targeted gene disruption, as well as markers and probes.

The invention also provides a method of plant breeding, e.g., to prepare a crossed fertile transgenic plant. The method comprises crossing a fertile transgenic plant comprising a particular nucleic acid molecule of the invention with itself or with a second plant, e.g., one lacking the particular nucleic acid molecule, to prepare the seed of a crossed fertile transgenic plant comprising the particular nucleic acid molecule. The seed is then planted to obtain a crossed fertile transgenic plant. The plant may be a monocot or a dicot. In a particular embodiment, the plant is a cereal plant.

The crossed fertile transgenic plant may have the particular nucleic acid molecule inherited through a female parent or through a male parent. The second plant may be an inbred plant. The crossed fertile transgenic may be a hybrid. Also included within the present invention are seeds of any of these crossed fertile transgenic plants.

Transformation of plants can be undertaken with a single DNA molecule or multiple DNA molecules (i.e., co-transformation), and both these techniques are suitable for use with the expression cassettes of the present invention. Numerous transformation vectors are available for plant transformation, and the expression cassettes of this invention can be used in conjunction with any such vectors. The selection of vector will depend upon the preferred transformation technique and the target species for transformation.

A variety of techniques are available and known to those skilled in the art for introduction of constructs into a plant cell host. These techniques generally include transformation with DNA employing A. tumefaciens or A. rhizogenes as the transforming agent, liposomes, PEG precipitation, electroporation, DNA injection, direct DNA uptake, microprojectile bombardment, particle acceleration, and the like (See, for example, EP 295959 and EP 138341) (see below). However, cells other than plant cells may be transformed with the expression cassettes of the invention. The general descriptions of plant expression vectors and reporter genes, and Agrobacterium and Agrobacterium-mediated gene transfer, can be found in Gruber et al. (1993).

Expression vectors containing genomic or synthetic fragments can be introduced into protoplasts or into intact tissues or isolated cells. Preferably expression vectors are introduced into intact tissue. General methods of culturing plant tissues are provided for example by Maki et al., (1993); and by Phillips et al. (1988). Preferably, expression vectors are introduced into maize or other plant tissues using a direct gene transfer method such as microprojectile-mediated delivery, DNA injection, electroporation and the like. More preferably expression vectors are introduced into plant tissues using the microprojectile media delivery with the biolistic device. See, for example, Tomes et al. (1995). The vectors of the invention can not only be used for expression of structural genes but may also be used in exon-trap cloning, or promoter trap procedures to detect differential gene expression in varieties of tissues, (Lindsey et al., 1993; Auch & Reth et al.).

It is particularly preferred to use the binary type vectors of Ti and Ri plasmids of Agrobacterium spp. Ti-derived vectors transform a wide variety of higher plants, including monocotyledonous and dicotyledonous plants, such as soybean, cotton, rape, tobacco, and rice (Pacciotti et al., 1985: Byrne et al., 1987; Sukhapinda et al., 1987; Lorz et al., 1985; Potrykus, 1985; Park et al., 1985: Hiei et al., 1994). The use of T-DNA to transform plant cells has received extensive study and is amply described (EP 120516; Hoekema, 1985; Knauf, et al., 1983; and An et al., 1985). For introduction into plants, the chimeric genes of the invention can be inserted into binary vectors as described in the examples.

Other transformation methods are available to those skilled in the art, such as direct uptake of foreign DNA constructs (see EP 295959), techniques of electroporation (Fromm et al., 1986) or high velocity ballistic bombardment with metal particles coated with the nucleic acid constructs (Kline et al., 1987, and U.S. Pat. No. 4,945,050). Once transformed, the cells can be regenerated by those skilled in the art. Of particular relevance are the recently described methods to transform foreign genes into commercially important crops, such as rapeseed (De Block et al., 1989), sunflower (Everett et al., 1987), soybean (McCabe et al., 1988; Hinchee et al., 1988; Chee et al., 1989; Christou et al., 1989; EP 301749), rice (Hiei et al., 1994), and corn (Gordon Kamm et al., 1990; Fromm et al., 1990).

Those skilled in the art will appreciate that the choice of method might depend on the type of plant, i.e., monocotyledonous or dicotyledonous, targeted for transformation. Suitable methods of transforming plant cells include, but are not limited to, microinjection (Crossway et al., 1986), electroporation (Riggs et al., 1986), Agrobacterium-mediated transformation (Hinchee et al., 1988), direct gene transfer (Paszkowski et al., 1984), and ballistic particle acceleration using devices available from Agracetus, Inc., Madison, Wis. And BioRad, Hercules, Calif. (see, for example, Sanford et al., U.S. Pat. No. 4,945,050; and McCabe et al., 1988). Also see, Weissinger et al., 1988; Sanford et al., 1987 (onion); Christou et al., 1988 (soybean); McCabe et al., 1988 (soybean); Datta et al., 1990 (rice); Klein et al., 1988 (maize); Klein et al., 1988 (maize); Klein et al., 1988 (maize); Fromm et al., 1990 (maize); and Gordon-Kamm et al., 1990 (maize); Svab et al., 1990 (tobacco chloroplast); Koziel et al., 1993 (maize); Shimamoto et al., 1989 (rice); Christou et al., 1991 (rice); European Patent Application EP 0 332 581 (orchardgrass and other Pooideae); Vasil et al., 1993 (wheat); Weeks et al., 1993 (wheat). In one embodiment, the protoplast transformation method for maize is employed (European Patent Application EP 0 292 435, U.S. Pat. No. 5,350,689).

In another embodiment, a nucleotide sequence of the present invention is directly transformed into the plastid genome. Plastid transformation technology is extensively described in U.S. Pat. Nos. 5,451,513, 5,545,817, and 5,545,818, in PCT application no. WO 95/16783, and in McBride et al., 1994. The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate orthologous recombination with the plastid genome and thus allow the replacement or modification of specific regions of the plastome. Initially, point mutations in the chloroplast 16S rRNA and rps 12 genes conferring resistance to spectinomycin and/or streptomycin are utilized as selectable markers for transformation (Svab et al., 1990; Staub et al., 1992). This resulted in stable homoplasmic transformants at a frequency of approximately one per 100 bombardments of target leaves. The presence of cloning sites between these markers allowed creation of a plastid targeting vector for introduction of foreign genes (Staub et al., 1993). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3N-adenyltransferase (Svab et al., 1993). Other selectable markers useful for plastid transformation are known in the art and encompassed within the scope of the invention. Typically, approximately 15-20 cell division cycles following transformation are required to reach a homoplastidic state. Plastid expression, in which genes are inserted by orthologous recombination into all of the several thousand copies of the circular plastid genome present in each plant cell, takes advantage of the enormous copy number advantage over nuclear-expressed genes to permit expression levels that can readily exceed 10% of the total soluble plant protein. In a preferred embodiment, a nucleotide sequence of the present invention is inserted into a plastid targeting vector and transformed into the plastid genome of a desired plant host. Plants homoplastic for plastid genomes containing a nucleotide sequence of the present invention are obtained, and are preferentially capable of high expression of the nucleotide sequence.

Agrobacterium tumefaciens cells containing a vector comprising an expression cassette of the present invention., wherein the vector comprises a Ti plasmid, are useful in methods of making transformed plants. Plant cells are infected with an Agrobacterium tumefaciens as described above to produce a transformed plant cell, and then a plant is regenerated from the transformed plant cell. Numerous Agrobacterium vector systems useful in carrying out the present invention are known.

For example, vectors are available for transformation using Agrobacterium tumefaciens. These typically carry at least one T-DNA border sequence and include vectors such as pBIN19 (Bevan, 1984). In one preferred embodiment, the expression cassettes of the present invention may be inserted into either of the binary vectors pCIB200 and pCIB2001 for use with Agrobacterium. These vector cassettes for Agrobacterim-mediated transformation wear constructed in the following manner. PTJS75kan is created by NarI digestion of pTJS75 (Schmidhauser & Helinski, 1985) allowing excision of the tetracycline-resistance gene, followed by insertion of an AccI fragment from pUC4K carrying an NPTII (Messing & Vierra, 1982; Bevan et al., 1983; McBride et al., 1990). XhoI linkers are ligated to the EcoRV fragment of pCIB7 which contains the left and right T-DNA borders, a plant selectable nos/nptlI chimeric gene and the pUC polylinker (Rothstein et al., 1987), and the XhoI-digested fragment is cloned into SalI-digested pTJS75kan to create pCIB200 (see also EP 0 332 104, example 19). PCIB200 contains the following unique polylinker restriction sites: EcoRI, SstI, KpnI, BglII, XbaI, and SalI. The plasmid pCIB2001 is a derivative of pCIB200 which is created by the insertion into the polylinker of additional restriction sites. Unique restriction sites in the polylinker of pCIB2001 are EcoRI, SstI, KpnI, BglIl, XbaI, SalI, MluI, BclI, AvrII, ApaI, HpaI, and StuI. PCIB2001, in addition to containing these unique restriction sites also has plant and bacterial kanamycin selection, left and right T-DNA borders for Agrobacterium-mediated transformation, the RK2-derived trfA function for mobilization between E. coli and other hosts, and the OriT and OriV functions also from RK2. The pCIB2001 polylinker is suitable for the cloning of plant expression cassettes containing their own regulatory signals.

An additional vector useful for Agrobacterium-mediated transformation is the binary vector pCIB 10, which contains a gene encoding kanamycin resistance for selection in plants, T-DNA right and left border sequences and incorporates sequences from the wide host-range plasmid pRK252 allowing it to replicate in both E. coli and Agrobacterium. Its construction is described by Rothstein et al., 1987. Various derivatives of pCIB10 have been constructed which incorporate the gene for hygromycin B phosphotransferase described by Gritz et al., 1983. These derivatives enable selection of transgenic plant cells on hygromycin only (pCIB743), or hygromycin and kanamycin (pCIB715, pCIB717).

Methods using either a form of direct gene transfer or Agrobacterium-mediated transfer usually, but not necessarily, are undertaken with a selectable marker which may provide resistance to an antibiotic (e.g., kanamycin, hygromycin or methotrexate) or a herbicide (e.g., phosphinothricin). The choice of selectable marker for plant transformation is not, however, critical to the invention.

For certain plant species, different antibiotic or herbicide selection markers may be preferred. Selection markers used routinely in transformation include the nptII gene which confers resistance to kanamycin and related antibiotics (Messing & Vierra, 1982; Bevan et al., 1983), the bar gene which confers resistance to the herbicide phosphinothricin (White et al., 1990, Spencer et al., 1990), the hph gene which confers resistance to the antibiotic hygromycin (Blochinger & Diggelmann), and the dhfr gene, which confers resistance to methotrexate (Bourouis et al., 1983).

One such vector useful for direct gene transfer techniques in combination with selection by the herbicide Basta (or phosphinothricin) is pCIB3064. This vector is based on the plasmid pCIB246, which comprises the CaMV 35S promoter in operational fusion to the E. coli GUS gene and the CaMV 35S transcriptional terminator and is described in the PCT published application WO 93/07278, herein incorporated by reference. One gene useful for conferring resistance to phosphinothricin is the bar gene from Streptomyces viridochromogenes (Thompson et al., 1987). This vector is suitable for the cloning of plant expression cassettes containing their own regulatory signals.

An additional transformation vector is pSOG35 which utilizes the E. coli gene dihydrofolate reductase (DHFR) as a selectable marker conferring resistance to methotrexate. PCR is used to amplify the 35S promoter (about 800 bp), intron 6 from the maize Adh1 gene (about 550 bp) and 18 bp of the GUS untranslated leader sequence from pSOG10. A 250 bp fragment encoding the E. coli dihydrofolate reductase type II gene is also amplified by PCR and these two PCR fragments are assembled with a SacI-PstI fragment from pBI221 (Clontech) which comprised the pUC19 vector backbone and the nopaline synthase terminator. Assembly of these fragments generated pSOG19 which contains the 35S promoter in fusion with the intron 6 sequence, the GUS leader, the DHFR gene and the nopaline synthase terminator. Replacement of the GUS leader in pSOG19 with the leader sequence from Maize Chlorotic Mottle Virus check (MCMV) generated the vector pSOG35. pSOG19 and pSOG35 carry the pUC-derived gene for ampicillin resistance and have HindIII, SphI, PstI and EcoRI sites available for the cloning of foreign sequences.

Transgenic plant cells are then placed in an appropriate selective medium for selection of transgenic cells which are then grown to callus. Shoots are grown from callus and plantlets generated from the shoot by growing in rooting medium. The various constructs normally will be joined to a marker for selection in plant cells. Conveniently, the marker may be resistance to a biocide (particularly an antibiotic, such as kanamycin, G418, bleomycin, hygromycin, chloramphenicol, herbicide, or the like). The particular marker used will allow for selection of transformed cells as compared to cells lacking the DNA which has been introduced. Components of DNA constructs including transcription cassettes of this invention may be prepared from sequences which are native (endogenous) or foreign (exogenous) to the host. By “foreign” it is meant that the sequence is not found in the wild-type host into which the construct is introduced. Heterologous constructs will contain at least one region which is not native to the gene from which the transcription-initiation-region is derived.

To confirm the presence of the transgenes in transgenic cells and plants, a variety of assays may be performed. Such assays include, for example, “molecular biological” assays well known to those of skill in the art, such as Southern and Northern blotting, in situ hybridization and nucleic acid-based amplification methods such as PCR or RT-PCR; “biochemical” assays, such as detecting the presence of a protein product, e.g., by immunological means (ELISAs and Western blots) or by enzymatic function; plant part assays, such as seed assays; and also, by analyzing the phenotype of the whole regenerated plant, e.g., for disease or pest resistance.

DNA may be isolated from cell lines or any plant parts to determine the presence of the preselected nucleic acid segment through the use of techniques well known to those skilled in the art. Note that intact sequences will not always be present, presumably due to rearrangement or deletion of sequences in the cell.

The presence of nucleic acid elements introduced through the methods of this invention may be determined by polymerase chain reaction (PCR). Using this technique discreet fragments of nucleic acid are amplified and detected by gel electrophoresis. This type of analysis permits one to determine whether a preselected nucleic acid segment is present in a stable transformant, but does not prove integration of the introduced preselected nucleic acid segment into the host cell genome. In addition, it is not possible using PCR techniques to determine whether transformants have exogenous genes introduced into different sites in the genome, i.e., whether transformants are of independent origin. It is contemplated that using PCR techniques it would be possible to clone fragments of the host genomic DNA adjacent to an introduced preselected DNA segment.

Positive proof of DNA integration into the host genome and the independent identities of transformants may be determined using the technique of Southern hybridization. Using this technique specific DNA sequences that are introduced into the host genome and flanking host DNA sequences can be identified. Hence the Southern hybridization pattern of a given transformant serves as an identifying characteristic of that transformant. In addition it is possible through Southern hybridization to demonstrate the presence of introduced preselected DNA segments in high molecular weight DNA, i.e., confirm that the introduced preselected DNA segment has been integrated into the host cell genome. The technique of Southern hybridization provides information that is obtained using PCR, e.g., the presence of a preselected DNA segment, but also demonstrates integration into the genome and characterizes each individual transformant.

It is contemplated that using the techniques of dot or slot blot hybridization which are modifications of Southern hybridization techniques one could obtain the same information that is derived from PCR, e.g., the presence of a preselected DNA segment.

Both PCR and Southern hybridization techniques can be used to demonstrate transmission of a preselected DNA segment to progeny. In most instances the characteristic Southern hybridization pattern for a given transformant will segregate in progeny as one or more Mendelian genes (Spencer et al., 1992); Laursen et al., 1994) indicating stable inheritance of the gene. The nonchimeric nature of the callus and the parental transformants (R₀) is suggested by germline transmission and the identical Southern blot hybridization patterns and intensities of the transforming DNA in callus, R₀ plants and R₁ progeny that segregated for the transformed gene.

Whereas DNA analysis techniques may be conducted using DNA isolated from any part of a plant, RNA may only be expressed in particular cells or tissue types and hence it will be necessary to prepare RNA for analysis from these tissues. PCR techniques may also be used for detection and quantitation of RNA produced from introduced preselected DNA segments. In this application of PCR it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species can also be determined using dot or slot blot Northern hybridizations. These techniques are modifications of Northern blotting and will only demonstrate the presence or absence of an RNA species.

While Southern blotting and PCR may be used to detect the preselected DNA segment in question, they do not provide information as to whether the preselected DNA segment is being expressed. Expression may be evaluated by specifically identifying the protein products of the introduced preselected DNA segments or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures may also be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed.

Very frequently the expression of a gene product is determined by evaluating the phenotypic results of its expression. These assays also may take many forms including but not limited to analyzing changes in the chemical composition, morphology, or physiological properties of the plant. Morphological changes may include greater stature or thicker stalks. Most often changes in response of plants or plant parts to imposed treatments are evaluated under carefully controlled conditions termed bioassays.

The present invention also provides a method to identify a nucleotide sequence that directs tissue-specific or tissue-preferential transcription combined with a mycorrhizal fungus- or phosphate-induced transcription of linked nucleic acid in the genome of a plant cell by contacting a probe of plant nucleic acid, e.g., cRNA from rice, isolated from various tissues of a plant, with a plurality of isolated nucleic acid samples on one or more, i.e., a plurality of, solid substrates so as to form a complex between at least a portion of the probe and a nucleic acid sample(s) having sequences that are structurally related to the sequences in the probe. Each sample comprises one or a plurality of oligonucleotides corresponding to at least a portion of a plant gene. Then complex formation is compared between samples contacted with a particular tissue, e.g., a seed-specific, probe and samples contacted with a different tissue, e.g., a non-seed specific probe, so as to determine which RNAs are expressed in the particular tissue of the plant. The probe and/or samples may be nucleic acid from a dicot or from a monocot.

The compositions of the invention include plant nucleic acid molecules, and the amino acid sequences for the polypeptides or partial-length polypeptides encoded by the nucleic acid molecule which comprises an open reading frame. These sequences can be employed to alter expression of a particular gene corresponding to the open reading frame by decreasing or eliminating expression of that plant gene or by overexpressing a particular gene product. Methods of this embodiment of the invention include stably transforming a plant with the nucleic acid molecule which includes an open reading frame operably linked to a promoter capable of driving expression of that open reading frame (sense or antisense) in a plant cell. By “portion” or “fragment”, as it relates to a nucleic acid molecule which comprises an open reading frame or a fragment thereof encoding a partial-length polypeptide having the activity of the full length polypeptide, is meant a sequence having at least 80 nucleotides, more preferably at least 150 nucleotides, and still more preferably at least 400 nucleotides. If not employed for expressing, a “portion” or “fragment” means at least 9, preferably 12, more preferably 15, even more preferably at least 20, consecutive nucleotides, e.g., probes and primers (oligonucleotides), corresponding to the nucleotide sequence of the nucleic acid molecules of the invention. Thus, to express a particular gene product, the method comprises introducing to a plant, plant cell, or plant tissue an expression cassette comprising a promoter linked to an open reading frame so as to yield a transformed differentiated plant, transformed cell or transformed tissue. Transformed cells or tissue can be regenerated to provide a transformed differentiated plant. The transformed differentiated plant or cells thereof preferably expresses the open reading frame in an amount that alters the amount of the gene product in the plant or cells thereof, which product is encoded by the open reading frame. The present invention also provides a transformed plant prepared by the method, progeny and seed thereof.

The invention further includes a nucleotide sequence which is complementary to one (hereinafter “test” sequence) which hybridizes under stringent conditions with a nucleic acid molecule of the invention as well as RNA which is transcribed from the nucleic acid molecule. When the hybridization is performed under stringent conditions, either the test or nucleic acid molecule of invention is preferably supported, e.g., on a membrane or DNA chip. Thus, either a denatured test or nucleic acid molecule of the invention is preferably first bound to a support and hybridization is effected for a specified period of time at a temperature of, e.g., between 55 and 70° C., in double strength citrate buffered saline (SC) containing 0.1% SDS followed by rinsing of the support at the same temperature but with a buffer having a reduced SC concentration. Depending upon the degree of stringency required such reduced concentration buffers are typically single strength SC containing 0.1% SDS, half strength SC containing 0.1% SDS and one-tenth strength SC containing 0.1% SDS.

Polynucleotides derived from the nucleic acid molecules of the present invention having any of the nucleotide sequences of SEQ ID NO: 1 to 13 and SEQ ID NO: 27 and SEQ ID NOs: 103 and 105 encoding a polypeptide that mediates phosphate uptake into the plant cell such as those given in SEQ ID NOs: 73 to 87 and further polynucleotides derived from the nucleic acid molecules having any of the nucleotide sequences of SEQ ID NO: 29 to 41 representing promoter sequences, are useful to detect the presence in a test sample of at least one copy of a nucleotide sequence containing the same or substantially the same sequence, or a fragment, complement, or variant thereof. The sequence of the probes and/or primers of the instant invention need not be identical to those provided in the Sequence Listing or the complements thereof. Some variation in probe or primer sequence and/or length can allow additional family members to be detected, as well as orthologous genes and more taxonomically distant related sequences. Similarly probes and/or primers of the invention can include additional nucleotides that serve as a label for detecting duplexes, for isolation of duplexed polynucleotides, or for cloning purposes.

Preferred probes and primers of the invention include isolated, purified, or recombinant polynucleotides containing a contiguous span of between at least 12 to at least 1000 nucleotides of any nucleotide sequence which is substantially similar, and preferably has at least between 70% and 99% sequence identity to any one of SEQ ID NOs: 1 to to 13 and SEQ ID NO: 27, and SEQ ID NOs: 103 and 105 encoding a polypeptide that mediates phosphate uptake into the plant cell and further nucleotides of any nucleotide sequence which is substantially similar, and preferably has at least between 70% and 99% sequence identity to any one of SEQ ID NO: 29 to 41 representing promoter sequences, or the complements thereof, with each individual number of nucleotides within this range also being part of the invention. Preferred are isolated, purified, or recombinant polynucleotides containing a contiguous span of at least 12, 15, 18, 20, 25, 30, 35, 40, 50, 60, 70, 80, 90, 100, 150, 200, 300, 400, 500, 750, or 1000 nucleotides of any nucleotide sequence which is substantially similar, and preferably has at least between 70% and 99%, sequence identity to any one of SEQ ID NOs: 1 to to 13 and SEQ ID NO: 27 and SEQ ID NOs: 103 and 105 encoding a polypeptide that mediates phosphate uptake into the plant cell and further nucleotides of any nucleotide sequence which is substantially similar, and preferably has at least between 70% and 99%, sequence identity to any one of SEQ ID NO: 29 to 41 and SEQ ID NOs: 103 and 105 representing promoter sequences, or the complements thereof. The appropriate length for primers and probes will vary depending on the application. For use as PCR primers, probes are 12-40 nucleotides, preferably 18-30 nucleotides long. For use in mapping, probes are 50 to 500 nucleotides, preferably 100-250 nucleotides long. For use in Southern hybridizations, probes as long as several kilobases can be used. The appropriate length for primers and probes under a particular set of assay conditions may be empirically determined by one of skill in the art.

The primers and probes can be prepared by any suitable method, including, for example, cloning and restriction of appropriate sequences and direct chemical synthesis by a method such as the phosphodiester method of Narang et aL (Meth Enzymol 68: 90 (1979)), the diethylphosphoramidite method, the triester method of Matteucci et al. (J Am Chem Soc 103: 3185 (1981)), or according to Urdea et al. (Proc Natl Acad 80: 7461 (1981)), the solid support method described in EP 0 707 592, or using commercially available automated oligonucleotide synthesizers.

Detection probes are generally nucleotide sequences or uncharged nucleotide analogs such as, for example peptide nucleotides which are disclosed in International Patent Application WO 92/20702, morpholino analogs which are described in U.S. Pat. Nos. 5,185,444, 5,034,506 and 5,142,047. The probe may have to be rendered “non-extendable” such that additional dNTPs cannot be added to the probe. Analogs are usually non-extendable, and nucleotide probes can be rendered non-extendable by modifying the 3′ end of the probe such that the hydroxyl group is no longer capable of participating in elongation. For example, the 3′ end of the probe can be functionalized with the capture or detection label to thereby consume or otherwise block the hydroxyl group. Alternatively, the 3′ hydroxyl group simply can be cleaved, replaced or modified so as to render the probe non-extendable.

Any of the polynucleotides of the present invention can be labeled, if desired, by incorporating a label detectable by spectroscopic, photochemical, biochemical, immunochemical, or chemical means. For example, useful labels include radioactive substances (³²P, ³⁵S, ³H, ¹²⁵I), fluorescent dyes (5-bromodesoxyuridine, fluorescein, acetylaminofluorene, digoxigenin) or biotin. Preferably, polynucleotides are labeled at their 3′ and 5′ ends. Examples of non-radioactive labeling of nucleotide fragments are described in the French patent No. FR-7810975 and by Urdea et al. (Nuc Acids Res 16:4937 (1988)). In addition, the probes according to the present invention may have structural characteristics such that they allow the signal amplification, such structural characteristics being, for example, branched DNA probes as described in EP 0 225 807.

A label can also be used to capture the primer so as to facilitate the immobilization of either the primer or a primer extension product, such as amplified DNA, on a solid support. A capture label is attached to the primers or probes and can be a specific binding member that forms a binding pair with the solid's phase reagent's specific binding member, for example biotin and streptavidin. Therefore depending upon the type of label carried by a polynucleotide or a probe, it may be employed to capture or to detect the target DNA. Further, it will be understood that the polynucleotides, primers or probes provided herein, may, themselves, serve as the capture label. For example, in the case where a solid phase reagent's binding member is a nucleotide sequence, it may be selected such that it binds a complementary portion of a primer or probe to thereby immobilize the primer or probe to the solid phase. In cases where a polynucleotide probe itself serves as the binding member, those skilled in the art will recognize that the probe will contain a sequence or “tail” that is not complementary to the target. In the case where a polynucleotide primer itself serves as the capture label, at least a portion of the primer will be free to hybridize with a nucleotide on a solid phase. DNA labeling techniques are well known in the art.

Any of the polynucleotides, primers and probes of the present invention can be conveniently immobilized on a solid support. Solid supports are known to those skilled in the art and include the walls of wells of a reaction tray, test tubes, polystyrene beads, magnetic beads, nitrocellulose strips, membranes, microparticles such as latex particles, sheep (or other animal) red blood cells, duracytes and others. The solid support is not critical and can be selected by one skilled in the art. Thus, latex particles, microparticles, magnetic or non-magnetic beads, membranes, plastic tubes, walls of microtiter wells, glass or silicon chips, sheep (or other suitable animal's) red blood cells and duracytes are all suitable examples. Suitable methods for immobilizing nucleotides on solid phases include ionic, hydrophobic, covalent interactions and the like. A solid support, as used herein, refers to any material that is insoluble, or can be made insoluble by a subsequent reaction. The solid support can be chosen for its intrinsic ability to attract and immobilize the capture reagent. Alternatively, the solid phase can retain an additional receptor that has the ability to attract and immobilize the capture reagent. The additional receptor can include a charged substance that is oppositely charged with respect to the capture reagent itself or to a charged substance conjugated to the capture reagent. As yet another alternative, the receptor molecule can be any specific binding member which is immobilized upon (attached to) the solid support and which has the ability to immobilize the capture reagent through a specific binding reaction. The receptor molecule enables the indirect binding of the capture reagent to a solid support material before the performance of the assay or during the performance of the assay. The solid phase thus can be a plastic, derivatized plastic, magnetic or non-magnetic metal, glass or silicon surface of a test tube, microtiter well, sheet, bead, microparticle, chip, sheep (or other suitable animal's) red blood cells, duracytes and other configurations known to those of ordinary skill in the art. The polynucleotides of the invention can be attached to or immobilized on a solid support individually or in groups of at least 2, 5, 8, 10, 12, 15, 20, or 25 distinct polynucleotides of the invention to a single solid support. In addition, polynucleotides other than those of the invention may be attached to the same solid support as one or more polynucleotides of the invention.

The polynucleotides of the invention that are expressed or repressed in response to environmental stimuli such as, for example, biotic or abiotic stress or treatment with chemicals or pathogens or at different developmental stages can be identified by employing an array of nucleic acid samples, e.g., each sample having a plurality of oligonucleotides, and each plurality corresponding to a different plant gene, on a solid substrate, e.g., a DNA chip, and probes corresponding to nucleic acid expressed in, for example, one or more plant tissues and/or at one or more developmental stages, e.g., probes corresponding to nucleic acid expressed in seed of a plant relative to control nucleic acid from sources other than seed. Thus, genes that are upregulated or downregulated in the majority of tissues at a majority of developmental stages, or upregulated or downregulated in one tissue such as in seed, can be systematically identified. The probes may also correspond to nucleic acid expressed in respone to a defined treatment such as, for example, a treatment with a variety of plant hormones or the exposure to specific environmental conditions involving, for example, an abiotic stress or exposure to light.

Specifically, labeled rice cRNA probes were hybridized to the rice DNA array, expression levels were determined by laser scanning and then rice genes were identified that had a particular expression pattern. The rice oligonucleotide probe array consists of probes from over 18,000 unique rice genes, which covers approximately 40-50% of the genome. This genome array permits a broader, more complete and less biased analysis of gene expression.

As described herein, GeneChip® technology was utilized to discover rice phosphate transporter genes that are preferentially (or exclusively) expressed in respone to a symbionitic fungus or phosphate availability and further in specific plant tissues such as, for example, the plant root, leaf or stem, or the plant flower.

Using this approach, 13 genes were identified, the expression of which was altered, e.g., specifically elevated or repressed, in in response to a symbiontic fungus or phosphate availability.

Consequently, the invention also deals with a method for detecting the presence of a polynucleotide including a nucleotide sequence which is substantially similar, and preferably has at least between 70% and 99% sequence identity to any one of SEQ ID NOs: 1 to to 13 and SEQ ID NO: 27, and SEQ ID NOs: 103 and 105 encoding a polypeptide that mediates phosphate uptake into the plant cell and further nucleotides of any nucleotide sequence which is substantially similar, and preferably has at least between 70% and 99% sequence identity to any one of SEQ ID NO: 29 to 41 representing promoter sequences, or a fragment or a variant thereof, or a complementary sequence thereto in a sample, the method including the following steps of:
(a) bringing into contact a nucleotide probe or a plurality of nucleotide probes which can hybridize with polynucleotide having a nucleotide sequence which is substantially similar, and preferably has at least between 70% and 99% sequence identity to any one of SEQ ID NOs: 1 to to 13 and SEQ ID NO: 27, and SEQ ID NOs: 103 and 105 encoding a polypeptide that mediates phosphate uptake into the plant cell and further nucleotides of any nucleotide sequence which is substantially similar, and preferably has at least between 70% and 99% sequence identity to any one of SEQ ID NO: 29 to 41 representing promoter sequences, or a fragment or a variant thereof, or a complementary sequence thereto and the sample to be assayed.
(b) detecting the hybrid complex formed between the probe and a nucleotide in the sample.

The invention further concerns a kit for detecting the presence of a polynucleotide including a nucleotide sequence which is substantially similar, and preferably has at least between 70% and 99% sequence identity to any one of SEQ ID NOs: 1 to to 13 and SEQ ID NO: 27, and SEQ ID NOs: 103 and 105 encoding a polypeptide that mediates phosphate uptake into the plant cell and further nucleotides of any nucleotide sequence which is substantially similar, and preferably has at least between 70% and 99% sequence identity to any one of SEQ ID NO: 29 to 41 representing promoter sequences, or a fragment or a variant thereof, or a complementary sequence thereto in a sample, the kit including a nucleotide probe or a plurality of nucleotide probes which can hybridize with a nucleotide sequence included in a polynucleotide including a nucleotide sequence which is substantially similar, and preferably has at least between 70% and 99% sequence identity to any one of SEQ ID NOs: 1 to to 13 and SEQ ID NO: 27, and SEQ ID NOs: 103 and 105 encoding a polypeptide that mediates phosphate uptake into the plant cell and further nucleotides of any nucleotide sequence which is substantially similar, and preferably has at least between 70% and 99% sequence identity to any one of SEQ ID NO: 29 to 41 representing promoter sequences, or a fragment or a variant thereof, or a complementary sequence thereto and, optionally, the reagents necessary for performing the hybridization reaction.

In a first preferred embodiment of this detection method and kit, the nucleotide probe or the plurality of nucleotide probes are labeled with a detectable molecule. In a second preferred embodiment of the method and kit, the nucleotide probe or the plurality of nucleotide probes has been immobilized on a substrate.

The isolated polynucleotides of the invention can be used to create various types of genetic and physical maps of the genome of rice or other plants. Such maps are used to devise positional cloning strategies for isolating novel genes from the mapped crop species. The sequences of the present invention are also useful for chromosome mapping, chromosome identification, tagging of phosphate transporter genes.

The isolated polynucleotides of the invention can further be used as probes for identifying polymorphisms associated with phenotypes of interest such as, for example, enhanced phosphate utilization, and higher yield. Briefly, total DNA is isolated from an individual or isogenic line, cleaved with one or more restriction enzymes, separated according to mass, transferred to a solid support, and hybridized with a probe molecule according to the invention. The pattern of fragments hybridizing to a probe molecule is compared for DNA from different individuals or lines, where differences in fragment size signals a polymorphism associated with a particular nucleotide sequence according to the present invention. After identification of polymorphic sequences, linkage studies can be conducted. After identification of many polymorphisms using a nucleotide sequence according to the invention, linkage studies can be conducted by using the individuals showing polymorphisms as parents in crossing programs. Recombinants, F₂ progeny recombinants or recombinant inbreds, can then be analyzed using the same restriction enzyme/hybridization procedure. The order of DNA polymorphisms along the chromosomes can be inferred based on the frequency with which they are inherited together versus inherited independently. The closer together two polymorphisms occur in a chromosome, the higher the probability that they are inherited together. Integration of the relative positions of polymorphisms and associated marker sequences produces a genetic map of the species, where the distances between markers reflect the recombination frequencies in that chromosome segment. Preferably, the polymorphisms and marker sequences are sufficiently numerous to produce a genetic map of sufficiently high resolution to locate one or more loci of interest.

The use of recombinant inbred lines for such genetic mapping is described for rice (Oh et al., Mol Cells 8:175 (1998); Nandi et al., Mol Gen Genet 255:1 (1997); Wang et al., Genetics 136:1421 (1994)), sorghum (Subudhi et al., Genome 43:240 (2000)), maize (Burr et al., Genetics 118:519 (1998); Gardiner et al., Genetics 134:917 (1993)), and Arabidopsis (Methods in Molecular Biology, Martinez-Zapater and Salinas, eds., 82:137-146, (1998)). However, this procedure is not limited to plants and can be used for other organisms such as yeast or other fungi, or for oomycetes or other protistans.

The nucleotide sequences of the present invention can also be used for simple sequence repeat identification, also known as single sequence repeat, (SSR) mapping. SSR mapping in rice has been described by Miyao et al. (DNA Res 3:233 (1996)) and Yang et al. (Mol Gen Genet 245:187 (1994)), and in maize by Ahn et al. (Mol Gen Genet 241:483 (1993)). SSR mapping can be achieved using various methods. In one instance, polymorphisms are identified when sequence specific probes flanking an SSR contained within an sequence of the invention are made and used in polymerase chain reaction (PCR) assays with template DNA from two or more individuals or, in plants, near isogenic lines. A change in the number of tandem repeats between the SSR-flanking sequence produces differently sized fragments (U.S. Pat. No. 5,766,847). Alternatively, polymorphisms can be identified by using the PCR fragment produced from the SSR-flanking sequence specific primer reaction as a probe against Southern blots representing different individuals (Refseth et al., Electrophoresis 18:1519 (1997)). Rice SSRs were used to map a molecular marker closely linked to a nuclear restorer gene for fertility in rice as described by Akagi et al. (Genome 39:205 (1996)).

The nucleotide sequences of the present invention can be used to identify and develop a variety of microsatellite markers, including the SSRs described above, as genetic markers for comparative analysis and mapping of genomes. The nucleotide sequences of the present invention can be used in a variation of the SSR technique known as inter-SSR (ISSR), which uses microsatellite oligonucleotides as primers to amplify genomic segments different from the repeat region itself (Zietkiewicz et al., Genomics 20:176 (1994)). ISSR employs oligonucleotides based on a simple sequence repeat anchored or not at their 5′- or 3′-end by two to four arbitrarily chosen nucleotides, which triggers site-specific annealing and initiates PCR amplification of genomic segments which are flanked by inversely orientated and closely spaced repeat sequences. In one embodiment of the present invention, microsatellite markers derived from the nucleotide sequences disclosed in the Sequence Listing, or substantially similar sequences or allelic variants thereof, may be used to detect the appearance or is disappearance of markers indicating genomic instability as described by Leroy et al. (Electron. J Biotechnol, 3(2), at http://www.ejb.org (2000)), where alteration of a fingerprinting pattern indicated loss of a marker corresponding to a part of a gene involved in the regulation of cell proliferation. Microsatellite markers derived from nucleotide sequences as provided in the Sequence Listing will be useful for detecting genomic alterations such as the change observed by Leroy et al. (Electron. J Biotechnol, 3(2), supra (2000)) which appeared to be the consequence of microsatellite instability at the primer binding site or modification of the region between the microsatellites, and illustrated somaclonal variation leading to genomic instability. Consequently, the nucleotide sequences of the present invention are useful for detecting genomic alterations involved in somaclonal variation, which is an important source of new phenotypes.

In addition, because the genomes of closely related species are largely syntenic (that is, they display the same ordering of genes within the genome), these maps can be used to isolate novel alleles from wild relatives of crop species by positional cloning strategies. This shared synteny is very powerful for using genetic maps from one species to map genes in another. For example, a gene mapped in rice provides information for the gene location in maize and wheat.

The various types of maps discussed above can be used with the nucleotide sequences of the invention to identify Quantitative Trait Loci (QTLs) for a variety of uses, including marker-assisted breeding. Many important crop traits are quantitative traits and result from the combined interactions of several genes. These genes reside at different loci in the genome, often on different chromosomes, and generally exhibit multiple alleles at each locus. Developing markers, tools, and methods to identify and isolate the QTLs involved in Pi uptake and utilization, enables marker-assisted breeding to enhance Pi uptake and utilization or suppress undesirable traits that interfere with an efficient Pi uptake and utilization. The nucleotide sequences as provided in the Sequence Listing can be used to generate markers, including single-sequence repeats (SSRs) and microsatellite markers for QTLs involved in Pi uptake and utilization to assist marker-assisted breeding. The nucleotide sequences of the invention can be used to, identify QTLs involved in Pi uptake and utilization and isolate alleles as described by Li et al. in a study of QTLs involved in resistance to a pathogen of rice. (Li et al., Mol Gen Genet 261:58 (1999)). In addition to isolating QTL alleles in rice, other cereals, and other monocot and dicot crop species, the nucleotide sequences of the invention can also be used to isolate alleles from the corresponding QTL(s) of wild relatives. Transgenic plants having various combinations of QTL alleles can then be created and the effects of the combinations measured. Once an ideal allele combination has been identified, crop improvement can be accomplished either through biotechnological means or by directed conventional breeding programs. (Flowers et al., J Exp Bot 51:99 (2000); Tanksley and McCouch, Science 277:1063 (1997)).

In another embodiment the nucleotide sequences of the invention can be used to help create physical maps of the genome of maize, Arabidopsis and related species. Where the nucleotide sequences of the invention have been ordered on a genetic map, as described above, then the nucleotide sequences of the invention can be used as probes to discover which clones in large libraries of plant DNA fragments in YACs, PACs, etc. contain the same nucleotide sequences of the invention or similar sequences, thereby facilitating the assignment of the large DNA fragments to chromosomal positions. Subsequently, the large BACs, YACs, etc. can be ordered unambiguously by more detailed studies of their sequence composition and by using their end or other sequence to find the identical sequences in other cloned DNA fragments (Mozo et al., Nat Genet 22:271 (1999)). Overlapping DNA sequences in this way allows assembly of large sequence contigs that, when sufficiently extended, provide a complete physical map of a chromosome. The nucleotide sequences of the invention themselves may provide the means of joining cloned sequences into a contig, and are useful for constructing physical maps.

In another embodiment, the nucleotide sequences of the present invention may be useful in mapping and characterizing the genomes of other cereals. Rice has been proposed as a model for cereal genome analysis (Havukkala, Curr Opin Genet Devel 6:711 (1996)), based largely on its smaller genome size and higher gene density, combined with the considerable conserved gene order among cereal genomes (Ahn et al., Mol Gen Genet 241:483 (1993)). The cereals demonstrate both general conservation of gene order (synteny) and considerable sequence homology among various cereal gene families. This suggests that studies on the functions of phosphate transporter genes or proteins from rice could lead to elucidation of the functions of orthologous genes or proteins in other cereals, including maize, wheat, secale, sorghum, barley, millet, teff, milo, triticale, flax, gramma grass, Tripsacum sp., and teosinte. The nucleotide sequences according to the invention can also be used to physically characterize homologous chromosomes in other cereals, as described by Sarma et al. (Genome 43:191 (2000)), and their use can be extended to non-cereal monocots such as sugarcane, grasses, and lilies.

Given the synteny between rice and other cereal genomes, the nucleotide sequences of the present invention can be used to obtain molecular markers for mapping and, potentially, for positional cloning. Kilian et al. described the use of probes from the rice genomic region of interest to isolate a saturating number of polymorphic markers in barley, which were shown to map to syntenic regions in rice and barley, suggesting that the nucleotide sequences of the invention derived from the rice genome would be useful in positional cloning of syntenic phosphate transporter genes of interest from other cereal species. (Kilian, et al., Nucl Acids Res 23:2729 (1995); Kilian, et al., Plant Mol Biol 35:187 (1997)). Synteny between rice and barley has recently been reported in the area of the carrying malting quality QTLs (Han, et al., Genome 41:373 (1998)), and use of synteny between cereals for positional cloning efforts is likely to add considerable value to rice genome analysis. Likewise, mapping of the ligules region of sorghum was facilitated using molecular markers from a syntenic region of the rice genome. (Zwick, et al., Genetics 148:1983 (1998)).

Rice marker technology utilizing the nucleotide sequences of the present invention can also be used to identify QTL alleles for enhanced Pi uptake and utilization from a wild relative of cultivated rice, for example as described by Xiao, et al. (Genetics 150:899 (1998)). Wild relatives of domesticated plants represent untapped pools of genetic resources for abiotic and biotic stress resistance, apomixis and other breeding strategies, plant architecture, determinants of yield, secondary metabolites, and other valuable traits. In rice, Xiao et al. (supra) used molecular markers to introduce an average of approximately 5% of the genome of a wild relative, and the resulting plants were scored for phenotypes such as plant height, panicle length and 1000-grain weight. Trait-improving alleles were found for all phenotypes except plant height, where any change is considered negative. Of the 35 trait-improving alleles, Xiao et al. found that 19 had no effect on other phenotypes whereas 16 had deleterious effects on other traits. The nucleotide sequences of the invention such as those provided in the Sequence Listing can be employed as molecular markers to identify QTL alleles for enhanced Pi uptake and utilization from a wild relative, by which these valuable traits can be introgressed from wild relatives using methods including, but not limited to, that described by Xiao et al. ((1998) supra). Accordingly, the nucleotide sequences of the invention can be employed in a variety of molecular marker technologies for yield improvement.

Following the procedures described above to identify polymorphisms, and using a plurality of the nucleotide sequences of the invention, any individual (or line) can be genotyped. Genotyping a large number of DNA polymorphisms such as single nucleotide polymorphisms (SNPs), in breeding lines makes it possible to find associations between certain polymorphisms or groups of polymorphisms, and certain phenotypes. In addition to sequence polymorphisms, length polymorphisms such as triplet repeats are studied to find associations between polymorphism and phenotype. Genotypes can be used for the identification of particular cultivars, varieties, lines, ecotypes, and genetically modified plants or can serve as tools for subsequent genetic studies of complex traits involving multiple phenotypes.

The patent publication W095/35505 and U.S. Pat. Nos. 5,445,943 and 5,410,270 describe scanning multiple alleles of a plurality of loci using hybridization to arrays of oligonucleotides. The nucleotide sequences of the invention are suitable for use in genotyping techniques useful for each of the types of mapping discussed above.

In a preferred embodiment, the nucleotide sequences of the invention are useful for identifying and isolating a least one unique stretch of protein-encoding nucleotide sequence. The nucleotide sequences of the invention are compared with other coding sequences having sequence similarity with the sequences provided in the Sequence Listing, using a program such as BLAST. Comparison of the nucleotide sequences of the invention with other similar coding sequences permits the identification of one or more unique stretches of phosphate transproter coding sequence that are not identical to the corresponding coding sequence being screened. Preferably, a unique stretch of coding sequence of about 25 base pairs (bp) long is identified, more preferably 25 bp, or even more preferably 22 bp, or 20 bp, or yet even more preferably 18 bp or 16 bp or 14 bp. In one embodiment, a plurality of nucleotide sequences is screened to identify unique coding sequences accroding to the invention. In one embodiment, one or more unique coding sequences accroding to the invention can be applied to a chip as part of an array, or used in a non-chip array system. In a further embodiment, a plurality of unique coding sequences accroding to the invention is used in a screening array. In another embodiment, one or more unique coding sequences accroding to the invention can be used as immobilized or as probes in solution. In yet another embodiment, one or more unique coding sequences accroding to the invention can be used as primers for PCR. In a further embodiment, one or more unique coding sequences accroding to the invention can be used as organism-specific primers for PCR in a solution containing DNA from a plurality of sources.

In another embodiment unique stretches of nucleotide sequences according to the invention are identified that are preferably about 30 bp, more preferably 50 bp or 75 bp, yet more preferably 100 bp, 150 bp, 200 bp, 250, 500 bp, 750 bp, or 1000 bp. The length of an unique coding sequence may be chosen by one of skill in the art depending on its intended use and on the characteristics of the nucleotide sequence being used. In one embodiment, unique coding sequences accroding to the invention may be used as probes to screen libraries to find homologs, orthologs, or paralogs. In another embodiment, unique coding sequences accroding to the invention may be used as probes to screen genomic DNA or cDNA to find homologs, orthologs, or paralogs. In yet another embodiment, unique coding sequences accroding to the invention may be used to study gene evolution and genome evolution.

A computer readable medium containing one or more of the nucleotide sequences of the invention as well as methods of use for the computer readable medium are provided. This medium allows a nucleotide sequence corresponding to at least one of SEQ ID NOs: 29 to 42 (promoters), SEQ ID NOs: 1 to 13 and 27; 103 and 105 (open reading frames or fragments thereof), to be used as a reference sequence to search against a database. This medium also allows for computer-based manipulation of a nucleotide sequence corresponding to at least one of SEQ ID NOs: 1 to 13, 27, 103 and 105; and 29 to 42. Storage and use of nucleic acid sequences on a computer readable medium is well known in the art. (See for example U.S. Pat. Nos. 6,023,659; 5,867,402; 5,795,716) Examples of such medium include, but are not limited to, magnetic tape, optical disk, CD-ROM, random access memory, volatile memory, non-volatile memory and bubble memory. Accordingly, the nucleic acid sequences contained on the computer readable medium may be compared through use of a module that receives the sequence information and compares it to other sequence information. Examples of other sequences to which the nucleic acid sequences of the invention may be compared include those maintained by the National Center for Biotechnology Information (NCBI)(http://www.ncbi.nlm.nih.gov/) and the Swiss Protein Data Bank. A computer is an example of such a module that can read and compare nucleic acid sequence information. Accordingly, the invention also provides the method of comparing a nucleic acid sequence of the invention to another sequence. For example, a sequence of the invention may be submitted to the NCBI for a Blast search as described herein where the sequence is compared to sequence information contained within the NCBI database and a comparison is returned. The invention also provides nucleic acid sequence information in a computer readable medium that allows the encoded polypeptide to be optimized for a desired property. Examples of such properties include, but are not limited to, increased or decreased: thermal stability, chemical stability, hydrophylicity, hydrophobicity, and the like. Methods for the use of computers to model polypeptides and polynucleotides having altered activities are well known in the art and have been reviewed. (Lesyng et al., 1993; Surles et al., 1994; Koehl et al., 1996; Rossi et al., 2001).

In one embodiment, the present invention provides the means and tools for modulating the uptake and distribution of phosphorous in photosynthetic organisms such as plants. For example, utilizing plants adapted to low phosphorous conditions by targeting and modulating transporter genes involved in the uptake, accumulation and translocation of PI can benefit agriculture and commercial industry in numerous ways.

Excessive amounts of phosphorous, seen particularly in areas of intense animal cultivation, disrupt ecological balance and threaten ecosystems with overload as the surplus moves into water systems, creating eutrophication and degradation. In many parts of the United States and Europe, soluble phosphorous levels exceed crop requirements. In some instances the topical application of phosphorous has been four times actual requirements because of phosphate binding with other soil elements.

Thus, in an embodiment applicable to all of the above stated provisions, the present invention provides the ability to regulate nucleotide sequences encoding at least one polypeptide involved in the uptake, transport and/or translocation of phosphorous, as well as any polypeptides encoded thereby, or any antigene sequences thereof, which have numerous applications using techniques that are known to those skilled in the art of molecular biology, biotechnology, biochemistry, genetics, physiology or pathology. These techniques include the use of nucleotide molecules as hybridization probes, for chromosome and gene mapping, in PCR technologies, in the production of sense or antisense nucleic acids, in screening for new therapeutic molecules, in production of plants and seeds having desirable, inheritable, commercially useful phenotypes, or in discovery of inhibitory compounds.

In a further embodiment, the present invention provides the ability to modulate phosphate uptake into the plant, by over-expressing, under-expressing or knocking out one or more of the phosphate transporter genes provided within the scope of the present invention, particularly those given in SEQ ID NOs: 1 to 13, 27, 103 and 105 or their gene products, in a plant cell, in vitro or in planta. Expression vectors comprising at least one nucleotide sequence involved in phosphate uptake and transport, or any antigene sequences thereof, operably linked to at least one suitable promoter and/or regulatory sequence can be used to study the role of polypeptides encoded by said sequences, for example by transforming a host cell with said expression vector and measuring the effects of overexpression and underexpression of sequences. A host cell transformed with at least one expression vector comprising such nucleotide sequences, operably linked to suitable promoters and/or regulatory sequences, can be useful to produce a dietary supplement comprising a polypeptide having a defined amino acid profile.

In a further embodiment, the present invention provides a transformed plant host cell, or one obtained through breeding, capable of over-expressing, under-expressing, or having a knock out of amino acid genes and/or their gene products. The plant cell is transformed with at least one such expression vector wherein the plant host cell can be used to regenerate plant tissue or an entire plant, or seed there from, in which the effects of expression, including overexpression or underexpression, of the introduced sequence or sequences can be measured in vitro or in plants. The invention thus provides transgenic plants which show an increased phosphate uptake as compared to the corresponding naturally-occurring or wild type (native) plant. Alternatively, plants can be produced in which phosphate uptake is reduced at particular stages in the lifespan of the plant or in particular cells and tissues.

Further, the identification of temporal or spatially regulated/induced promoters and putative regulatory elements that are not only organ-specifically expressed, but also responsive to the presentce of nutrients or symbiontic fungi should allow for designing tools for transgenic strategies in cereals.

For example, the tissue specific expression of individual phosphate transporter genes provides a tool for designing strategies of enhanced phosphate utilization/translocation. In one specific embodiment, overexpression of the preferentially green-tissue- (leaf- and stem-) expressed phosphate transporter of the invention may lead to an increase in biomass of leaves which hence, may be of possible value in forage production

In another embodiment of the invention modulation of phosphate uptake and translocation in plant tissues and organs, respectively, may lead to an advance or delay in maturity, depending on the phosphate concentration in the flower. Modulating of these pathways may also lead to improved resistance to plant diseases and pathogens.

The phosphate transporter genes according to the invention or a homologue thereof can be introduced into a plant to modulated the phosphate uptake of the plant in response to specific soil conditions which might be different from those to which the parent plant is adapted. For example, a high affinity transporter of the invention can be incorporated into a plant which has been bred for other traits (e.g., high yield and disease resistance), to produce a plant that can obtain additional phosphate from its environment.

The phosphate transporter genes according to the invention can be stacked with other isolated or recombinant nucleic acids which preferentially modify phosphate transport pathways which are present in tissues of all parts of the plant, or which are present in actively growing tissues or in storage tissues or organs such as seeds to provide increased photosynthetic capacity or to provide mechanisms for disease or stress resistance.

Further, the isolated nucleic acids of the invention which comprise a promoter sequence that is inducible under conditions of phosphate deficiency or sufficiency or in response to a mycorrhizal fungus or a fragment or functional equivalent thereof which initiates the transcription of at least one nucleic acid which encodes a polypeptide for production of a useful product of interest. Expression of nucleic acid compositions according to the invention result is the large scale and inexpensive production of valuable proteins or other products in a particular plant tissue upon induction by increasing or decreasing the level of available phosphate or by growing the plant in the presence of a symbionitic fungus.

These plants can be grown in conventional hydroponic culture media to provide an efficient system by which the plants can be deprived of phosphate to induce protein synthesis. Further, using a hydroponic culture system permits quick and efficient harvesting of roots and other plant parts for isolation of products.

Further, the promoter of the invention can be linked to a nucleic acid molecule encoding a protein which functions as a rate limiting enzyme in a metabolic pathway, thus providing a means for interfering with a regulatory function for the plant in response to varying phosphate levels or the presence of a symbiontic fungus. This would allow, for example, correlating the production of a product with a specific developmental stage of the plant. The product could be a harvestable compound or it could have a regulatory function, such as flower evocation.

Those promoters of the invention that are in addition specifically or preferentially expressed in a specific tissue or organ of the plant such as roots, green-tisse (leaf- or stem-) or flowers, are also useful to induce tissue abundant expression of a structural gene of interest to which it is operably linked.

The phosphate transporter genes, in combination with the promoter sequences of the present invention driving the expression of disease or pathogen resistance genes, either endogenous or foreign, can further be used to protect plants from diseases and pests to which the plant is most susceptible under conditions of low available phosphate (less than 1 mM exogenous inorganic phosphate). For example, while the high-affinity and phosphate deficiency-inducible phosphate transporter genes according to the invention, under phosphate-deficient conditions, initiate additional phosphate uptake into the plant, thus strengthening the plant's own resistance mechanisms, an additional disease or pathogen resistance gene under the control of a phosphate-deficiency inducible promoter induces expression of a plant protective protein such as, for example, an insecticidal toxin or an antifungal protein which would further help a crop or horticultural plant to prevent or resist an insect or fungal attack.

The promoter of this invention can be particularly useful for controlling nematode attack. Nematodes are primitive eukaryotic root parasites that live in the soil where they puncture plant roots and suck the cellular contents, weakening the plant and providing an entry point for pathogenic fungi and bacteria. It is known in the art that phosphate-deficiency promoters in plant roots are responsive to nematode invasion. For example, beta-glucosidase (such as psr3.2) expression is shown to be increased when nematodes attack roots, suggesting that changes in exogenous inorganic phosphate regulation are part of the plant's resistance mechanism to nematode injury.

The phosphate deficiency promoter of the present invention may thus be used to control nematod injury in plants by linking it to a gene encoding a nematode toxin, so that, under nematode attack, the toxin is expressed locally when and where it is required. This would be an efficient and cost-effective way of controlling plant nematodes.

It is known in the art that relatively high phosphate levels advances maturity in plants, whereas relatively low phosphate levels results in little or no flowering taking place. The genes and promoter sequences provided in this invention can thus also be used to decrease or increase the vegetative phase (and therefore shorten or lengthen the time to flowering) of crops.

To decrease the vegetative phase, introducing additional copies of a phosphate-deficiency inducible promoter linked to high-affinity transporter gene of the invention or any other regulatory gene whose expression results in uptake of additional phosphate from the environment into the plant could increase the phosphate to nitrogen ratio in the plant. This, in turn, can cause floral induction and earlier flowering.

For plants harvested for their vegetative parts such as stems and leaves (e.g., lettuce, cabbage, spinach, maize) the duration of the vegetative phase ought to be increased. In that case, a phosphate-deficiency inducible promoter is linked to an antisense sequences to a high-affinity transporter gene of the invention or to any other endogenous regulatory or structural gene as described above and expressed in the plant. Under limiting phosphate conditions, expression of the antisense gene could inhibit transcription or translation of the endogenous high-affinity transporter gene or a regulatory gene, thereby preventing increased uptake of phosphate and increasing the ratio of nitrogen to phosphorus in the plant. The result would be to increase yield of these crops by delaying flowering.

### EXAMPLES

The invention will be further described by reference to the following detailed examples. These examples are provided for purposes of illustration only, and are not intended to be limiting unless otherwise specified. Standard recombinant DNA and molecular cloning techniques used here are well known in the art and are described in detail in Sambrook et al. (Molecular Cloning: A Laboratory Manual, Cold Spring Harbor Laboratory Press (1989)) and by Ausubel et al. (Current Protocols in Molecular Biology, Greene Publishing (1992)).

### Example 1

### Plant Growth Conditions

Oryza sativa cv. Nipponbare plants are grown in sterilized soil in phytochambers with a 12-h/12-h light/dark regime at 28° C., ˜50% relative humidity and a light intensity of 300 μEi. Tissue samples are harvested at various developmental stages. For studies of the mycorrhizal symbiosis, rice seeds are surface sterilized in 3.5% sodium hypochlorite for 10 min, followed by 5 cycles of extensive wash in ddH₂O. Sand is chosen as a low-fertility soil substrate to facilitate root harvest, the assessment of the root system and determination of the degree of mycorrhizal colonization. Plants are watered every second day with half-strength Hoagland solution supplemented with KH₂PO₄ concentrations of 5--500 μM.

### Example 2

### Plant Inoculation

2.1 Inoculation of Plants with Glomus intraradices

Glomus intraradices is grown in aseptic conditions and spores for plant inoculum are prepared according to Becard and Fortin (1987). For plant inoculation, 10 ml of G. intraradices growth medium containing 1000 spores is added to the sand at 1.5 cm depth just before planting. Ten seeds per pot are planted at 0.5 cm depth. Equal volumes of medium without fungal spores are added to the mock-inoculated samples.

2.2 Rice Inoculation with Rhizoctonia solani and Fusarium moniliforme

Sterilized rice seeds are germinated on potato dextrose agar (PDA, Difco, Detroit) for 3 days at 24° C. in the dark and subsequently transferred to Magenta boxes (Polylabo, Strasbourg, France), each containing 150 ml of M medium without sucrose but supplemented with glucose (1 g/l) (Bécard and Fortin (1987). The pH of the medium is adjusted to 5.5 with KOH and the medium is solidified with 0.4% (w/v) phytagel (Sigma, St. Louis, Mo., USA).

Infection with R. solani AG 1-IA and F. moniliforme, which had been maintained on potato dextrose agar medium (PDA, Difco Laboratories), is carried out on 10-day-old rice seedlings. Roots approximately 1 cm long are inoculated with pieces of solidified medium containing mycelium. The plants are grown further in Magenta boxes at 24° C. (12-h day/night cycle) for an additional 8 days and roots are collected for microscopic inspection and RNA extraction.

### Example 3

### Microscopic Examination of Mycorrhiza and Root Pathogens

The degree of mycorrhizal colonization is determined after trypan blue staining of root samples at various times after inoculation. The number of intraradical fungal structures is determined as described in Brundrett at al. (1984) using the gridline intersect procedure according to Giovanetti and Mosse (1980).

To estimate the degree of root infection by R. solani and F. moniliforme, mock- and fungus-inoculated tissue is fixed in 3% glutaraldehyde in 0.1 M sodium cacodylate buffer pH 7.2 for 2 h at room temperature and washed in the same buffer. Small pieces of root (approximately 2 mm³) are dehydrated in a series of ethanol washes and embedded in LR White resin (Jauneau et al., 1997). Sections of approximately 1 μm are cut with a diamond knife on a Reichert Ultracut E microtome (Leica, Germany) and stained with toluidine blue. Root samples are examined under a stereomicroscope (Wild M5A) or an inverted microscope (Leitz DMIRBE, Leica). Images are documented with a CCD camera (Colour Coolview, Photonic Science) and processed by Image Pro Plus (Media Cybernetics Md., USA).

### Example 4

### Identification of Rice Pi Transporters

The amino acid sequence of AtPT1 (Muchhal et al., 1996, AAB17265) is used to search the rice genome by TBLASTN (Altschul et al., 1997). The identified AtPt1 homologues and their neighboring regions are examined by various gene prediction programs (Burge and Karlin, 1997; Lukashin and Borodovsky, 1998; Salamov and Solovyev, 1999). The ClustaW program (Thompson, 1994) is used for multiple sequence alignments. DNA sequences are analyzed for possible motifs by internally developed programs based on standard programs and are also examined for redundancies by comparing to a internal database of rice repeats.

In addition, the identification of the rice genes provides a means to identify the corresponding homologs and orthologs in other plants, including commercially valuable food crops such as wheat, banana, and maize. The rice sequences can be compared to sets of clustered cDNA's from other cereal species such as, for example, wheat, maize and banana using the same software process to retrieve the top hits from each set which revealed the polynucleotide sequences set forth in the Sequence Listing (see Table 1).

The comparison algorithm can then be used such as, for example, a translated BLAST search, tblastx. The BLAST results can be post processed using an appropriate software such as, for example, SCAN software with the default parameters. This processed data may then be parsed to retrieve the top rice hit based on E-value. These rice sequences can then be compared to sets of clustered cDNA's from other cereal species such as, for example, wheat, banana and maize using the same software process to retrieve the top hits from each set.

TABLE 1
Rice Phosphate Transporter Genes (OsPHT) and orthologous
sequences in wheat, banana and maize.

| | Wheat | Banana | Maize |
|---|---|---|---|
| | Ortholog | Ortholog | Ortholog |
| OsPHT | SEQ ID | SEQ ID | SEQ ID |
| (SEQ ID NO) | NO | NO | NO |
| 1; 1 (1) | 98 | 94 | 99 |
| 1; 2 (2) | 95 | 94 | 99 |
| 1; 3 (3) | 97 | 94 | 99 |
| 1; 4 (4) | 98 | 94 | 100 |
| 1; 5 (5) | 98 | 94 | 102 |
| 1; 6 (6) | 98 | 94 | 102 |
| 1; 7 (7) | 98 | 94 | 102 |
| 1; 8 (8) | 97 | 94 | 99 |
| 1; 9 (9) | 98 | 94 | 99 |
| 1; 10 (10) | 98 | 94 | 99 |
| 1; 11 (11) | 95 | 94 | 102 |
| 1; 12 (12) | 98 | 94 | 99 |
| 1; 13 (13) | 95 | 94 | 102 |
| 2; 1 (27) | 96 | | 101 |

### Example 5

### RNA Isolation and Analysis

Total RNA is isolated from 1 g fresh weight of plant tissue using the RNeasy Maxi Kit (Qiagen, Hilden, Germany). DNaseI-treated RNA (10 ng) is used with TaqMan One-Step RT-PCR reagents and quantitatively analyzed in an ABI 7700 (Applied Biosystems, Foster City, Calif., USA). Reactions are carried out as described by the manufacturers. Primers and 6-FAM 5′ end-labeled probes (Applied Biosystems and Genosys, The Woodlands, Tex., USA) are designed for the 3′UTR of all Pi transporter genes and the rice polyubiquitin 1 gene (RubQ1) mRNA as a control (RubQ1, Wang et al., 2000), employing the PRIMER EXPRESS software (Applied Biosystems):

| | |
|---|---|
| forward primer | reverse primer |

| | | | | |
|---|---|---|---|---|
| OsPHT1.1 | CGCTTCCGTACGAGTGGTAGT | (SEQ ID NO: 58) | GGTTCTTTCAAATCCAGGGAAA | (SEQ ID NO: 43) |
| OsPHT1.2 | GACAGACCGCCCAAGAAG | (SEQ ID NO: 59) | TTTTCAGTCACTCACGTCGAGAC | (SEQ ID NO: 44) |
| OsPHT1.3 | TGCGACTGCTGATTCAGTACGT | (SEQ ID NO: 60) | ACAAATGCCATCAAATATGAACAG | (SEQ ID NO: 45) |
| | | | A | |
| OsPHT1.4 | TTCTGCTAGTGTACCAAACAAA | (SEQ ID NO: 61) | GTAAGTGGCATTTATAATATCAACA | (SEQ ID NO: 46) |
| | ATTACA | | GTAACC | |
| OsPHT1.5 | TGCTACTGCCCATGACTAGGATT | (SEQ ID NO: 62) | CCATAGAAGAGATCCAGAGAAGCT | (SEQ ID NO: 47) |
| | | | GTA | |
| OsPHT1.6 | CCGCCCCTGCAAACTGTA | (SEQ ID NO: 63) | GAACTGGCGGTTTCTTCGAT | (SEQ ID NO: 48) |
| OsPHT1.7 | GCAAGTCGCTCGAGGAGATG | (SEQ ID NO: 64) | TGGAATTAACGGGTGGATCAC | (SEQ ID NO: 49) |
| OsPHT1.8 | AGAAGGCAAAAGAAATGTGTGT | (SEQ ID NO: 65) | AAAATGTATTCGTGCCAAATTGCT | (SEQ ID NO: 50) |
| | TAAAT | | | |
| OsPHT1.9 | AGAAAAACATAGGCTTGTCATC | (SEQ ID NO: 66) | AAAACCTAAGAAGCACTGTAAATA | (SEQ ID NO: 51) |
| | CTTT | | AATCC | |
| OsPHT1.10 | ATGTCGCCCATCCTTCCA | (SEQ ID NO: 67) | TCGCTTTCCGACGATGATC | (SEQ ID NO: 52) |
| OsPHT1.11 | GAGAAGTTCCCTGCTTCAAGCA | (SEQ ID NO: 68) | TGCATATCCCAGATGAGCGTATC | (SEQ ID NO: 53) |
| OsPHT1.12 | AACGAGATGACGAACACTTGCA | (SEQ ID NO: 69) | TCCTGTACCTAAAAAGCAAGTACT | (SEQ ID NO: 54) |
| | | | AACATAGT | |
| OsPHT1.13 | TTAGCATAATTCTTTTAGTAGTT | (SEQ ID NO: 70) | TGATTTAAGATAAGGATTGAATGC | (SEQ ID NO: 55) |
| | AAATAGGAGATG | | ACAT | |
| OsPHT2.1 | TGTACAGCCCATTGCACTTCA | (SEQ ID NO: 71) | AATTTTATGGTCGAAACTTTTTGGA | (SEQ ID NO: 56) |
| RubQ1 | GGGTTCACAAGTCTGCCTATTTG | (SEQ ID NO: 72) | ACGGGACACGACCAAGGA | (SEQ ID NO: 57) |

Probe

| | | |
|---|---|---|
| OsPHT1.1 | CACCAATTCTTCAAGAACATATACACACAT | (73) |
| OsPHT1.2 | GATCGCCCAAGCGTGATGTCATAAACAT | (SEQ ID NO: 74) |
| OsPHT1.3 | CATTTGGACACTGCCATTCACATCACACT | (SEQ ID NO: 75) |
| OsPHT1.4 | TACTAGTCGTCAACCCAGGCAACGCA | (SEQ ID NO: 76) |
| OsPHT1.5 | CAACATGGTGAAAATCTGCTGGTTCCCA | (SEQ ID NO: 77) |
| OsPHT1.6 | TAGCGCTGTCGCCGTCTGC | (SEQ ID NO: 78) |
| OsPHT1.7 | CTGGCCGACCTCGTTCTCCCGT | (SEQ ID NO: 79) |
| OsPHT1.8 | TCTGCAAGAATTATTCCTCAAGTGGCCAAA | (SEQ ID NO: 80) |
| OsPHT1.9 | CGTTTCCGCTCCTCCTGCCAATC | (SEQ ID NO: 81) |
| OsPHT1.10 | AGTTCACTCACACGGAGACCCGGG | (SEQ ID NO: 82) |
| OsPHT1.11 | CGAATGGCAGACATCCATGCACG | (SEQ ID NO: 83) |
| OsPHT1.12 | TTCCTATCAGGCTACAAGTACGAGCTTCGC | (SEQ ID NO: 84) |
| OsPHT1.13 | TTGGTGGCAGATTTCTATCCGTGACACTC | (SEQ ID NO: 85) |
| OsPHT2.1 | TCAGCCTATCTCTTTGCCATTGCGGAA | (SEQ ID NO: 86) |
| RubQ1 | CAGACCAGACAACCATAGCTCCATTGGG | (SEQ ID NO: 87) |

Expression levels are calculated using standard curves with a correlation coefficient of ≧0.99. The amount of transcript for each gene is normalized to the expression level of the RubQ1 gene. We examined RubQ1 gene expression in a set of three independent experiments under a broad range of environmental stimuli and various developmental stages using the Affymetrix rice GeneChip. The results of these experiments suggested constitutive RubQ1 expression (data not shown). This is exemplified by the constant levels of RubQ1 mRNA within a time-course experiment, where the relative levels of RubQ1 expression after 2, 4 and 6 weeks in mock-inoculated roots are 2198, 1804 and 2642, respectively, and for the corresponding mycorrhizal roots are 2557, 2612 and 2241.

### Example 6

### Computational Identification of the Complete Set of Rice High Affinity-like Pi Transporters

The Arabidopsis AtPT1 protein (Muchhal et al., 1996; AAB17265, AT5g43360) is used as a query sequence for a TBLASTN search (Altschul et al., 1990) on the rice genome to identify high affinity Pi transporters. A total of thirteen high affinity Pi transporter related full length sequences with scores <E−20 and containing all domains predicted to be required for the function of the phosphate transporter are identified. In order to determine the gene and cDNA structures of each Pi transporter and also to translate their protein sequences, gene prediction programs are applied to the genomic regions containing transporter genes. The resulting identified genes are termed according to the Commission on Plant Gene Nomenclature (http://mbclserver.rutgers.edu/CPGN/Guide.html) ORYsa;Pht1;1 through ORYsa;Pht1;13 but they will be referred to as OsPHT1;1 through OsPHT1;13 and OsPHT2;1 herein. The thirteen high affinity transporter proteins are found to share 44.3% to 74% sequence identity with AtPT1 and 37.9% to 85.9% among each other. The highest degree of sequence identity is found between OsPHT1;1 and OsPHT1;3. In contrast 98.9% sequence identity can be seen among individual members of the Arabidopsis thaliana high affinity transporter genes. Also for Medicago truncatula, sequence identities as high as to 98% have been reported (Liu et al., 1998). Thus, the rice Pi transporter family is more divergent than those of the two dicotyledonous species. However, in analogy to the Pi transporter proteins from dicotyledonous plants the predicted rice proteins contain the conserved sequence motifs for phosphorylation sites for protein kinase C (Thr-Ala-Arg motif, between position 200 and 248 of SEQ ID NOs: 14-26 and 28 and 104) and casein kinase II (Ser-Leu-Glu-Glu(Val;Asp), between postion 456 and 521 of SEQ ID NOs: 14-26 and 28 and 104) as well as for potential N-glycosylation (Asn-Ser(Ala;Thr)-Thr between postion 383 and 433 of SEQ ID NOs: 14-26; reviewed in Smith et al., 2000).

The transgenome comparison revealed that Arabidopsis and rice varied in their total number of high affinity Pi transporter related genes. While Arabidopsis possessed 9 genes (http://www.cbs.umn.edu/Arabidopsis) we identified 13 for rice suggesting that there may be a general difference in how rice and Arabidopsis coordinate Pi acquisition and translocation. A clear grouping of mono- and dicotyledonous Pi transporter-related proteins is also observed upon analyzing the phylogenetical relationship of the rice proteins and dicotyledonous high affinity Pi transporter proteins. While the dicotyledonous proteins clustered with members of the respective same species as well as with members of rather distant other dicotyledonous plants (e.g. AT3g54700 clustered with At2g38940 and the three distant solanaceous LePT1, StPT1 and NtPT1) most of the rice proteins remained clustered with other rice members and not with those of dicotyledonous species. OsPHT1;11 displayed the most extreme divergence and did neither assemble well with any given other plant Pi transporter protein nor any of the known fungal ones.

### Example 7

### Colonization of Oryza saliva cv. Nipponbare by Glomus intraradices

Since there is no information available on mycorrhizal rice, efficient colonization of rice roots by G. intraradices had to be established. Eight weeks old sand grown rice plant displayed a fully developed symbiosis with G. intraradices. The roots contained all typical fungal structures like intercellular hyphae, arbuscules and vesicles and reached colonization levels of 85 (±9%). The plants had been fertilized with a reduced amount of Pi (50 μM KH₂PO₄) promoting efficient colonization of their roots by mycorrhizal fungi. In order to verify whether or not the AM symbiosis had led to enhanced Pi uptake the total P content of roots and shoots of mycorrhizal and non-mycorrhizal control plants is determined (as ppm per gram (g) dryweight (dw) of roots and leaves of plants grown at 50 μM Pi). Roots of mycorrhizal plants contained 2.31 (±0.5) ppm P/g dw while roots of control plants had 1.23 (±0.4) ppm P/g dw. A similar ratio is obtained for the leave tissue, leaves of mycorrhizal plants exhibited a total P content of 2.89 (±0.59) ppm P/g dw and non-mycorrhizal leaves reached 1.84 (±0.28) ppm P/g dw (data obtained from 3 independent experiments). Both types of organs, roots as well as leaves, showed increased levels of total P when the plant had formed the AM symbiosis.

### Example 8

### Expression Analysis of Pi Transporter on Mycorrhizal and Mock Inoculated Rice Roots

In order to identify Pi transporter transcriptionally regulated upon mycorrhizal colonization gene specific primers and probes are designed against the region of the computationally predicted 3′ untranslated region (UTR) and TaqMan real time PCR is used to monitor transcript levels. To ensure specificity the primer and probe sequences are reblasted against the rice genome. The rice ubiquitin 1 gene (RubQ1) is used as a constitutively expressed control

Expression analysis revealed that ten out of the thirteen Pi transporter are expressed in roots. Transcripts of OsPHT1;12 and OsPHT1;13 are not and of OsPHT1;7 only barely detectable. This could be due to mistakes in gene prediction leading to wrongly designed primer and probe sequences or alternatively could reflect that these genes are either pseudogenes or low or not expressed, respectively, in roots. Transcripts of OsPHT1;1, OsPHT1;2, OsPHT1;3, OsPHT1;6, OsPHT1;9 and OsPHT1;10 accumulate in both mock and G. intraradices inoculated roots, but all of them are 1.5-3 fold downregulated upon mycorrhizal colonization. Therefore this group of Pi transporters genes is not a preferred set for involvement in the Pi uptake at the plant-fungus interface. For Pi transporter genes OsPHT1;4, OsPHT1;5 and OsPHT1;8 expression did not change significantly upon the presence of G. intraradices and hence, their possible participation in fungus mediated Pi uptake also less preferred. Preferred (with respect ot AM mediated uptake) OsPHT1;11 appeared transcriptionally silent in mock inoculated roots but is strongly induced upon mycorrhizal colonization. Transcript accumulation occurred only in roots and is not detected in leaves of mycorrhizal plants.

### Example 9

### OsPHT1;2 and OsPHT1;11 Expression at Different Pi Availability

It could be speculated that the enhanced Pi supply provided by the fungus accounted for the activation of OsPHT1;11. For addressing this question expression studies are carried out on rice plants grown for six weeks at increasing levels of Pi. In order to define Pi limiting versus sufficient conditions two approaches are chosen. The first make use of Pi starvation inducible high affinity Pi transporter genes as a molecular marker for limiting Pi availability. The second determines the total Pi content of roots and shoots to examine the correlation between varying Pi application and Pi status of the plants.

Presently known root-expressed high affinity Pi transporter from dicotyledonous plants exhibit transcripts levels strongly elevated upon Pi starvation. For instance the two Pi transporter, MtPT1 and MtPT2, identified from Medicago truncatula have been shown to be not only Pi starvation inducible but also downregulated upon mycorrhizal colonization (Liu et al., 1998). OsPHT1;2 is chosen as a candidate gene to be possibly regulated in a similar fashion and in fact displayed the expected expression pattern with high transcript levels at 5, 25 and 100 μM Pi and lower levels at 500 μM Pi allowing for its application as a molecular indicator of restricted Pi availability.

Assessing the Pi content of roots and leaves results in similarly low levels of total Pi at 5, 25 and 100 μM Pi (1.2, 1.2 and 1.6 ppm Pi/g dw for roots and 2.2, 1.8 and 1.8 ppm Pi/g dw for leaves). The addition of 500 μM Pi increases the Pi values by approximately 3 fold to 4.1 and 6.1 ppm Pi/g dw for roots and leaves, respectively. The comparable values obtained at the lower concentrations of 5, 25 and 100 μM Pi indicate that the plants had grown at restricted Pi availability under the conditions applied while the elevated levels of P content at 500 μM Pi reflect Pi sufficiency. These data match well with those of the OsPHT1;2 expression.

Strikingly, transcripts of OsPHT1;11 are below the limit of detection at all Pi concentrations reflecting that gene activation occurs independently on the availability of Pi and the Pi status of the plant.

### Example 10

### Developmental and Organ Specific Expression of OsPHT1;2 and OsPHT1;11

In order to determine whether or not OsPHT1;2 and OsPHT1;11 expression occurrs in other plant organs at different developmental stages RNA is isolated from various stages of the root, leaf, stem, panicle, seed and of embryo and endosperm tissue (for roots, shoots and stems tissue is harvested at germination, booting and maturation stage and for shoots and stems in addition at senescence stage. Two stages each are collected for flowers, booting and panicle emergence, and also for seed development, milk and dough stage. Last, embryos are separately used from endosperm.). There is no expression of OsPHT1;2 in any aerial tissue, neither leaves, stems, seeds, embryo or endosperm. However, high levels of transcript are observed in roots of different developmental stage being most pronounced in roots of mature plants. This elevated expression in mature roots could be either due to developmental regulation or due to the rapidly formed depletion zone around the roots leading to Pi starvation and hence, induction of this gene.

Expression of OsPHT1;11 can not be monitored in any of the given organs or tissues at any developmental stage.

### Example 11

### Temporal Expression of OsPHT1;11

To further examine the correlation between mycorrhizal colonization and gene activation, induction of the OsPHT1;11 messenger is followed over time intervals of 15, 25, 35, 45 and 55 dpi (days post inoculation). Microscopic determination of the degree of colonization revealed that rare intraradical structures (2±1.4%) could be monitored at 25 dpi (days post inoculation) followed by clearly detectable 25±8.5% at 35 dpi and a rapid increase to 56±7.1% at 45 dpi reaching 83±5.7% at 55 dpi. OsPHT1;11 messenger is not detectable in Mock inoculated roots and also not at 15 dpi (E−03). A moderate signal is monitored at 25 dpi (4.74E−02), which increased slightly at 35 dpi (8.02E−02) and is followed by a strong induction of more than one order of magnitude at 45 dpi (1.50E+00) and a further slower enhancement at 55 dpi (2.68E+00). Therefore the kinetics of OsPHT1;11 gene expression correlated well with the amount of fungus present within the roots.

### Example 12

### Expression of OsPHT1;11 in Interactions with Pathogenic Fungi

Since the obtained data indicate that OsPHT1;11 is specifically regulated in response to colonization by the AM fungus G. intraradices, the plants are challenged with another pathogenic fungus in order to investigate the specificity of the OsPHT1;11 gene activation. For this purpose rice roots are inoculated with Rhizoctonia solani. Necrosis symptoms appeared on stem and leaves 8 days after inoculation (data not shown). Microscopic inspection at infection sites revealed that R. solani had penetrated and grown inside the root tissue and had invaded cortex cells. Following the expression of OsPHT1;11 on pathogen infected rice roots showed that despite of having high amounts of RubQ1 mRNA no OsPHT1;11 transcript accumulation could be detected. Induction of the OsPHT1;11 gene is therefore monitored exclusively in the presence of the AM fungus G. intraradices.

### Example 13

### Cloning and Sequencing of Nucleic Acid Molecules from Rice Tissue

Plant genomic DNA samples can be isolated from frozen tissues, according to one of the three procedures, e.g., standard procedures described by Ausubel et al. (1995), a quick leaf prep described by Klimyuk et al. (1993), or using FTA paper (Life Technologies). For the latter procedure, a piece of leaf is excised from the plant, placed on top of the FTA paper and covered with a small piece of parafilm that serves as a barrier material to prevent contamination of the crushing device. In order to drive the sap and cells from the plant tissue into the FTA paper matrix for effective cell lysis and nucleic acid entrapment, a crushing device is used to mash the tissue into the FTA paper. The FIA paper is air dried for an hour. For analysis of DNA, the samples can be archived on the paper until analysis. Two mm punches are removed from the specimen area on the FTA paper using a 2 mm Harris Micro Punch™ and placed into PCR tubes. Two hundred (200) microliters of FTA purification reagent is added to the tube containing the punch and vortexed at low speed for 2 seconds. The tube is then incubated at room temperature for 5 minutes. The solution is removed with a pipette so as to repeat the wash one more time. Two hundred (200) microliters of TE (10 mM Tris, 0.1 mM EDTA, pH 8.0) is added and the wash is repeated two more times. The PCR mix is added directly to the punch for subsequent PCR reactions.

### Example 14

### Cloning of the OsPHT1;11 cDNA and Genomic DNA

OsPHT1;11 cDNA is amplified from total RNA isolated from mycorrhizal rice roots after reverse transcription using primers designed against the computationally predicted cDNA. Primers designed based on the genomic sequence can be used to PCR amplify the full-length cDNA (start to stop codon) from first strand cDNA prepared from rice cultivar Nipponbare root tissue.

The Qiagen RNeasy kit (Qiagen, Hilden, Germany) is used for extraction of total RNA. The Superscript II kit (Invitrogen, Carlsbad, USA) is used for the reverse transcription reaction. PCR amplification of the OsPHT1;11 cDNA and genomic DNA is carried out using the reverse primer and sequence 5′ATGGGCTTCTTCACCGTCGCCTAC3′ located at the translation start of the gene. This is performed with high-fidelity Taq polymerase (Invitrogen, Carlsbad, USA).

The PCR fragment is then cloned into pCR2.1-TOPO (Invitrogen) or the pGEM-T easy vector (Promega Corporation, Madison, Wis., USA) per the manufacturer's instructions, and several individual clones are subjected to sequencing analysis.

DNA sequencing. DNA preps for 2-4 independent clones are miniprepped following the manufacturer's instructions (Qiagen). DNA is subjected to sequencing analysis using the BigDye⊥ Terminator Kit according to manufacturer's instructions (ABI). Sequencing makes use of primers designed to both strands of the predicted gene of interest. DNA sequencing is performed using standard dye-terminator sequencing procedures and automated sequencers (models 373 and 377; Applied Biosystems, Foster City, Calif.). All sequencing data are analyzed and assembled using the Phred/Phrap/Consed software package (University of Washington) to an error ratio equal to or less than 10⁻⁴ at the consensus sequence level.

The consensus sequence from the sequencing analysis is then to be validated as being intact and the correct gene in several ways. The coding region is checked for being full length (predicted start and stop codons present) and uninterrupted (no internal stop codons). Alignment with the gene prediction and BLAST analysis is used to ascertain that this is in fact the right gene.

The clones are sequenced to verify their correct amplification. While the amplified cDNA is 1668 bp, the genomic clone contained a small intron of 92 bp in length. The corresponding protein is predicted to consist of 555 aa and according to different topology prediction programs between 11 and 13 transmembrane domains (TMHMM: 11, HMMTOP: 13, DAS: 11) According to TMpred (Genetics Computer Group program package; Devreux et al., 1994) the preferred topology for the OsPHT1;11 protein contained 12 membrane spanning domains having an intracellular N- and C-terminus and also an intracellular long central loop. This topology is shared by other membrane transporters belonging to the major facilitator superfamily of proteins (reviewed in Smith et al., 2000). OsPHT1;11 displayed between 37.9%-56.1% with the other rice transporter proteins and between 39.8%-57.4% with the other publicly available Pi transporter proteins. On the basis of sequence comparison the OsPHT1;11 protein is therefore equivalenlty distant to any of the dicotyledonous Pi transporter proteins as it is to the other members of the rice high affinity transporter family This is also reflected by an isolated position in the phylogenetic tree since OsPHT1;11 did not cluster well with any of the presently known high affinity-like Pi transporter proteins as mentioned above.

The sequences are in agreement with gene prediction results. The cDNA of OsPHT1;11 is then inserted into the yeast expression vector p426 Adh (American Type Culture Collection, Manassas, Va., USA). The same primers are employed for the amplification of the genomic clone.

### Example 15

### Functional Analysis in pho84

For the functional characterization of OsPHT1;11 protein the yeast deletion mutant pho84 (Saccharomyces Genome Deletion Project, Stanford, http://sequence-www.stanford.edu/group/yeast_deletion_project/deletions3.html) being impaired in Pi uptake from low Pi media (Bun-ya et al., 1991) is transformed with either the OsPHT1;11 cDNA under the constitutive control of the alcohol dehydrogenase promoter or with the empty vector control. In ³²Pi uptake experiments the rate of transport is linear with time at 200 μM Pi during the first 8 min. The mutant cells expressing the OsPHT1;11 protein displayed 3.25 fold higher Pi acquisition than those cells carrying the empty vector control. The increased Pi uptake indicated that the OsPHT1;11 gene encodes a functional Pi transporter.

### Example 16

### Functional Analysis in Plants

An analogous approach to that described in Example 15 above can be used for the functional characterization of the OsPHTs in plants. Plant deletion mutants that are impaired in Pi uptake from media or soil depleted in Pi can be complemented by the respective rice gene.

Rice and Arabidopsis putative orthologue pairs are identified using BLAST comparisons, TFASTXY comparisons, and Double-Affine Smith-Waterman similarity searches. Constructs containing a rice cDNA or genomic clone inserted between the promoter and terminator of the Arabidopsis orthologue are generated using overlap PCR (Gene 77, 61-68 (1989)) and GATEWAY cloning (Life Technologies Invitrogen). For ease of cloning, rice cDNA clones are preferred to rice genomic clones. A three stage PCR strategy is used to make these constructs.

(1) In the first stage, primers are used to PCR amplify: (i) 2Kb upstream of the translation start site of the Arabidopsis orthologue, (ii) the coding region or cDNA of the rice orthologue, and (iii) the 500 bp immediately downstream of the Arabidopsis orthogue's translation stop site. Primers are designed to incorporate onto their 5′ ends at least 16 bases of the 3′ end of the adjacent fragment, except in the case of the most distal primers which flank the gene construct (the forward primer of the promoter and the reverse primer of the terminator). The forward primer of the promoters contains on their 5′ ends partial AttB1 sites, and the reverse primer of the terminators contains on their 5′ ends partial AttB2 sites, for Gateway cloning.

(2) In the second stage, overlap PCR is used to join either the promoter and the coding region, or the coding region and the terminator.

(3) In the third stage either the promoter-coding region product can be joined to the terminator or the coding region-terminator product can be joined to the promoter, using overlap PCR and amplification with fulll Att site-containing primers, to link all three fragments, and put full Att sites at the construct termini.

The fused three-fragment pieces flanked by Gateway cloning sites are introduced into the LTI donor vector pDONR201 (Invitrogen) using the BP clonase reaction, for confirmation by sequencing. Confirmed sequenced constructs are introduced into a binary vector containing Gateway cloning sites, using the LR clonase reaction such as, for example, pAS200.

The pAS200 vector was created by inserting the Gateway cloning cassette RfA into the Acc65I site of pNOV3510.

pNOV3510 was created by ligation of inverted pNOV2114 VSI binary into pNOV3507, a vector containing a PTX5′ Arab Protox promoter driving the PPO gene with the Nos terminator.

pNOV2114 contains a VS1 origin of replication and a copy of the Agrobacterium virG gene in the backbone and was created by insertion of virGN54D (Pazour et al. 1992, J. Bacteriol. 174:4169-4174) from pAD1289 (Hansen et al. 1994, PNAS 91:7603-7607) into pHiNK085.

pHiNK085 was created by deleting the 35S:PMI cassette and M13 ori in pVictorHiNK.

pPVictorHiNK was created by modifying the T-DNA of pVictor (described in WO 97/04112) to delete M13 derived sequences and to improve its cloning versatility by introducing the BIGLINK polylinker.

The sequence of the pVictor HiNK vector is disclosed in SEQ ID NO: 5 in WO 00/6837, which is incorporated herein by reference. The pVictorHiNK vector contains the following constituents that are of functional importance:
The origin of replication (ORI) functional in Agrobacterium is derived from the Pseudomonas aeruginosa plasmid pVS1 (Itoh et al. 1984. Plasmid 11: 206-220; Itoh and Haas, 1985. Gene 36: 27-36). The pVS1 ORI is only functional in Agrobacterium and can be mobilised by the helper plasmid pRK2013 from E. coli into A. tumefaciens by means of a triparental mating procedure (Ditta et al., 1980. Proc. Natl. Acad. Sci USA 77: 7347-7351).
The ColE1 origin of replication functional in E. coli is derived from pUC19 (Yannisch-Perron et al., 1985. Gene 33: 103-119).
The bacterial resistance to spectinomycin and streptomycin encoded by a 0.93 kb fragment from transposon Tn7 (Fling et al., 1985. Nucl. Acids Res. 13: 7095) functions as selectable marker for maintenance of the vector in E. coli and Agrobacterium The gene is fused to the tac promoter for efficient bacterial expression (Amman et al., 1983. Gene 25: 167-178).
The right and left T-DNA border fragments of 1.9 kb and 0.9 kb that comprise the 24 bp border repeats, have been derived from the Ti-plasmid of the nopaline type Agrobacterium tumefaciens strains pTiT37 (Yadav et al., 1982. Proc. Natl. Acad. Sci. USA. 79: 6322-6326).

The plasmid is introduced into Agrobacterium tumefaciens GV3101pMP90 by electroporation. The positive bacterial transformants are selected on LB medium containing 50 μg/μl kanamycin and 25 μg/μl gentamycin. Plants are transformed by standard methodology (e.g., by dipping flowers into a solution containing the Agrobacterium) except that 0.02% Silwet-77 Lehle Seeds, Round Rock, Tex.) is added to the bacterial suspension and the vacuum step omitted. Five hundred (500) mg of seeds are planted per 2 ft² flat of soil and, and progeny seeds are selected for transformants using PPO selection.

Primary transformants are analyzed for complementation. Primary transformants are genotyped for the Arabidopsis mutation and presence of the transgene. When possible, >50 mutants harboring the transgene should be phenotyped to observe variation due to transgene copy number and expression

### Example 17

### Vector Construction for Overexpression and Gene “Knockout” Experiments

Overexpression

Vectors used for expression of full-length “phosphate transporter candidate genes” of interest in plants (overexpression) are designed to overexpress the protein of interest and are of two general types, biolistic and binary, depending on the plant transformation method to be used.

For biolistic transformation (biolistic vectors), the requirements are as follows:
1. a backbone with a bacterial selectable marker (typically, an antibiotic resistance gene) and origin of replication functional in Escherichia coli (E. coli ; eg. ColE1), and
2. a plant-specific portion consisting of:
   a. a gene expression cassette consisting of a promoter (eg. ZmUBIint MOD), the gene of interest (typically, a full-length cDNA) and a transcriptional terminator (eg. Agrobacterium tumefaciens nos terminator);
   b. a plant selectable marker cassette, consisting of a promoter (eg. rice Act1D-BV MOD), selectable marker gene (eg. phosphomannose isomerase, PMI) and transcriptional terminator (eg. CaMV terminator).
      Vectors designed for transformation by Agrobacterium tumefaciens (A. tumefaciens; binary vectors) consist of:
1. a backbone with a bacterial selectable marker functional in both E. coli and A. tumefaciens (eg. spectinomycin resistance mediated by the aadA gene) and two origins of replication, functional in each of aforementioned bacterial hosts, plus the A. tumefaciens virG gene;
2. a plant-specific portion as described for biolistic vectors above, except in this instance this portion is flanked by A. tumefaciens right and left border sequences which mediate transfer of the DNA flanked by these two sequences to the plant.
   Knock Out Vectors

Vectors designed for reducing or abolishing expression of a single gene or of a family or related genes (knockout vectors) are also of two general types corresponding to the methodology used to downregulate gene expression: antisense or double-stranded RNA interference (dsRNAi).

Anti-Sense

For antisense vectors, a full-length or partial gene fragment (typically, a portion of the CDNA) can be used in the same vectors described for full-length expression, as part of the gene expression cassette. For antisense-mediated down-regulation of gene expression, the coding region of the gene or gene fragment will be in the opposite orientation relative to the promoter; thus, mRNA will be made from the non-coding (antisense) strand in planta.

dsRNAi

For dsRNAi vectors, a partial gene fragment (typically, 300 to 500 basepairs long) is used in the gene expression cassette, and is expressed in both the sense and antisense orientations, separated by a spacer region (typically, a plant intron, eg. the OsSH1 intron 1, or a selectable marker, eg. conferring kanamycin resistance). Vectors of this type are designed to form a double-stranded mRNA stem, resulting from the basepairing of the two complementary gene fragments in planta.

Biolistic or binary vectors designed for overexpression or knockout can vary in a number of different ways, including eg. the selectable markers used in plant and bacteria, the transcriptional terminators used in the gene expression and plant selectable marker cassettes, and the methodologies used for cloning in gene or gene fragments of interest (typically, conventional restriction enzyme-mediated or Gateway™ recombinase-based cloning). An important variant is the nature of the gene expression cassette promoter driving expression of the gene or gene fragment of interest in most tissues of the plants (constitutive, eg. ZmUBIint MOD), in specific plant tissues (eg. maize ADP-gpp for endosperm-specific expression), or in an inducible fashion (eg. GAL4bsBz1 for estradiol-inducible expression in lines constitutively expressing the cognate transcriptional activator for this promoter).

17.1. Insertion of a “Phosphate Transporter Candidate Gene” Into Expression Vector

A validated rice cDNA clone such as the OsPHT1;11 cDNA prepared in Example 14 above, in pCR2.1-TOPO is subcloned using conventional restriction enzyme-based cloning into a vector, downstream of the maize ubiquitin promoter and intron, and upstream of the Agrobacterium tumefaciens nos 3′ end transcriptional terminator. The resultant gene expression cassette (promoter, “phosphate transporter candidate gene” and terminator) is further subcloned, using conventional restriction enzyme-based cloning, into the pNOV2117 binary vector (Negrotto et al (2000) Plant Cell Reports 19,798-803; plasmid pNOV117 discosed in this article corresponds to pNOV2117 described herein; the nucleotide sequence of pNOV2117 is provided in SEQ ID NO: 44 of WO 01/73087), generating pNOVCAND.

The pNOVCAND binary vector is designed for transformation and over-expression of the “phosphate transporter candidate gene” in monocots. It consists of a binary backbone containing the sequences necessary for selection and growth in Escherichia coli DH-5a (Invitrogen) and Agrobacterium tumefaciens LBA4404 (pAL4404; pSB1), including the bacterial spectinomycin antibiotic resistance aada gene from E. coli transposon Tn7, origins of replication for E. coli (ColE1) and A. tumefaciens (VS 1), and the A. tumefaciens virG gene. In addition to the binary backbone, which is identical to that of pNOV2114 described herein previously (see Example 16 above), pNOV2117 contains the T-DNA portion flanked by the right and left border sequences, and including the Positech™ (Syngenta) plant selectable marker and the “phosphate transporter candidate gene” gene expression cassette. The Positech™ plant selectable marker confers resistance to mannose and in this instance consists of the maize ubiquitin promoter driving expression of the PMI (phosphomannose isomerase) gene, followed by the cauliflower mosaic virus transcriptional terminator.

Plasmid pNOV2117 is introduced into Agrobacterium tumefaciens LBA4404 (pAL404; pSB1) by electroporation. Plasmid pAL4404 is a disarmed helper plasmid (Ooms et al (1982) Plasmid 7, 15-29). Plasmid pSB1 is a plasmid with a wide host range that contains a region of homology to pNOV2117 and a 15.2 kb KpnI fragment from the virulence region of pTiBo542 (Ishida et al (1996) Nat Biotechnol 14, 745-750). Introduction of plasmid pNOV2117 into Agrobacterium strain LBA4404 results in a co-integration of pNOV2117 and pSB1.

Alternatively, plasmid pCIB7613, which contains the hygromycin phosphotransferase (hpt) gene (Gritz and Davies, Gene 25, 179-188, 1983) as a selectable marker, may be employed for transformation.

Plasmid pCIB7613 (see WO 98/06860, incorporated herein by reference in its entirety) is selected for rice transformation. In pCIB7613, the transcription of the nucleic acid sequence coding hygromycin-phosphotransferase (HYG gene) is driven by the corn ubiquitin promoter (ZmUbi) and enhanced by corn ubiquitin intron 1. The 3′ polyadenylation signal is provided by NOS 3′ nontranslated region.

Other useful plasmids include pNADII002 (GAL4-ER-VP16) which contains the yeast GAIA DNA Binding domain (Keegan et al., Science, 231:699 (1986)), the mammalian estrogen receptor ligand binding domain (Greene et al., Science, 231 :1150 (1986)) and the transcriptional activation domain of the HSV VP16 protein (Triezenberg et al.,1988). Both hpt and GAL4-ER-VP16 are constitutively expressed using the maize Ubiquitin promoter, and pSGCDL1 (GAL4BS Bz1 Luciferase), which carries the firefly luciferase reporter gene under control of a minimal maize Bronzel (Bz1) promoter with 10 upstream synthetic GAL4 binding sites. All constructs use termination signals from the nopaline synthase gene.

### Example 18

### Plant Transfornzation

18.1 Rice Transformation

pNOVCAND is transformed into a rice cultivar (Kaybonnet) using Agrobacterium-mediated transformation, and mannose-resistant calli are selected and regenerated.

Agrobacterium is grown on YPC solid plates for 2-3 days prior to experiment initiation. Agrobacterial colonies are suspended in liquid MS media to an OD of 0.2 at λ600 nm. Acetosyringone is added to the agrobacterial suspension to a concentration of 200 μM and agro is induced for 30 min.

Three-week-old calli which are induced from the scutellum of mature seeds in the N6 medium (Chu, C. C. et al., Sci, Sin., 18, 659-668(1975)) are incubated in the agrobacterium solution in a 100×25 petri plate for 30 minutes with occasional shaking. The solution is then removed with a pipet and the callus transfered to a MSAs medium which is overlayed with sterile filter paper.

Co-Cultivation is continued for 2 days in the dark at 22° C.

Calli are then placed on MS-Timetin plates for 1 week. After that they are tranfered to PAA+mannose selection media for 3 weeks.

Growing calli (putative events) are picked and transfered to PAA+mannose media and cultivated for 2 weeks in light.

Colonies are tranfered to MS20SorbKinTim regeneration media in plates for 2 weeks in light. Small plantlets are transferred to MS20SorbKinTim regeneration media in GA7 containers. When they reach the lid, they are transfered to soil in the greenhouse.

Expression of the “phosphate transporter candidate gene” in transgenic To plants is analyzed. Additional rice cultivars, such as but not limited to, Nipponbare, Taipei 309 and Fuzisaka 2 are also transformed and assayed for expression of the “phosphate transporter candidate gene” product and enhanced protein expression.

18.2 Maize Transfonnation

Transformation of immature maize embryos is performed essentially as described in Negrotto et al., (2000) Plant Cell Reports 19: 798-803. For this example, all media constituents are as described in Negrotto et al., supra. However, various media constituents described in the literature may be substituted.

1. Transformation Plasmids and Selectable Marker

The genes used for transformation are cloned into a vector suitable for maize transformation as described in Example 17. Vectors used contain the phosphomannose isomerase (PMI) gene (Negrotto et al. (2000) Plant Cell Reports 19: 798-803).

2. Preparation of Agrobacterium tumefaciens

Agrobacterium strain LBA4404 (pSB1) containing the plant transformation plasmid is grown on YEP (yeast extract (5 g/L), peptone (10 g/L), NaCl (5 g/L),15 g/l agar, pH 6.8) solid medium for 2 to 4 days at 28° C. Approximately 0.8×10⁹ Agrobacteria are suspended in LS-inf media supplemented with 100 μM acetosyringone (As) (Negrotto et al.,(2000) Plant Cell Rep 19: 798-803). Bacteria are pre-induced in this medium for 30-60 minutes.

3. Inoculation

Immature embryos from A188 or other suitable maize genotypes are excised from 8 -12 day old ears into liquid LS-inf+100 μM As. Embryos are rinsed once with fresh infection medium. Agrobacterium solution is then added and embryos are vortexed for 30 seconds and allowed to settle with the bacteria for 5 minutes. The embryos are then transferred scutellum side up to LSAs medium and cultured in the dark for two to three days. Subsequently, between 20 and 25 embryos per petri plate are transferred to LSDc medium supplemented with cefotaxime (250 mg/l ) and silver nitrate (1.6 mg/l and cultured in the dark for 28° C. for 10 days.

4. Selection of Transformed Cells and Regeneration of Transformed Plants

Immature embryos producing embryogenic callus are transferred to LSD1M0.5S medium. The cultures are selected on this medium for 6 weeks with a subculture step at 3 weeks. Surviving calli are transferred either to LSD1M0.5S medium to be bulked-up or to Reg1 medium. Following culturing in the light (16 hour light/8 hour dark regiment), green tissues are then transferred to Reg2 medium without growth regulators and incubated for 1-2 weeks. Plantlets are transferred to Magenta GA-7 boxes (Magenta Corp, Chicago Ill.) containing Reg3 medium and grown in the light. Plants that are PCR positive for the promoter-reporter cassette are transferred to soil and grown in the greenhouse.

### Example 19

### Promoter Analysis

The gene chip experiment described above in Example 5 are designed to uncover genes that are expressed in seed tissue during grain filling. Candidate promoters are identified based upon the expression profiles of the associated transcripts.

Candidate promoters are obtained by PCR and fused to a GUS reporter gene containing an intron. Both histochemical and fluormetric GUS assays are carried out on stably transformed rice and maize plants and GUS activity is detected in the transformants.

Further, transient assays with the promoter::GUS constructs are carried out in rice embryogenic callus and GUS activity is detected by histochemical staining according the protocol described below (see Example 20).

Construction of Binary Promoter::Reporter Plasmids

To construct a binary promoter:: reporter plasmid for rice transformation a vector containing a promoter of interest (i.e., the DNA sequence 5′ of the initiation codon for the gene of interest) is used, which results from recombination in a BP reaction between a PCR product using the promoter of interest as a template and pDONR201™, producing an entry vector. The regulatory/promoter sequence is fused to the GUS reporter gene (Jefferson et al, 1987) by recombination using GATEWAY™ Technology according to manufacturers protocol as described in the Instruction Manual (GATEWAY™ Cloning Technology, GIBCO BRL, Rockville, Md. http://www.lifetech.com/).

Briefly, the Gateway Gus-intron-Gus (GIG)/NOS expression cassette is ligated into pNOV2117 binary vector in 5′ to 3′ orientation. The 4.1 kB expression cassette is ligated into the Kpn-I site of pNOV2117, then clones are screened for orientation to obtain pNOV2346, a GATEWAY™ adapted binary destination vector.

For example, the promoter fragment of SEQ ID NO: 42 is provided in the entry vector and recombined via the LR reaction with the binary destination vector containing the GUS coding region with an intron that has an attR site 5′ to the GUS reporter, producing a binary vector with a promoter fused to the GUS reporter (pNOV6070). The orientation of the inserted fragment is maintained by the att sequences and the final construct is verified by sequencing. The construct is then transformed into Agrobacterium tumefaciens strains by electroporation as described herein previously (see Example 17.1).

### Example 20

### Transient Expression Analysis of Candidate Promoters in Rice Embryogenic Callus

Materials:

Embryogenic rice callus (Kaybonett cultivar)
LBA 4404 Agrobacterium strains
KCMS liquid media for re-suspending bacterial pellet
200 mM stock (40 mg/ml) Acetosyringone
Sterile filter paper discs (8.5 mm in diameter)
LB spec liquid culture
MS-CIM media plates
MS-AS plates (co-cultivation plates)
MS-Tim plates (recovery plates)
Gus staining solution
   Methods:
   Induction of Embryogenic Callus:
1. Sterilize mature Kaybonett rice seeds in 40% ultra Clorox, 1 drop Tween 20, for 40 min.
2. Rinse with sterile water and plate on MS-CIM media (12 seeds/plate)
3. Grow in dark for four weeks.
4. Isolate embryogenic calli from scutellum to MS-CIM
5. Let grow in dark 8 days before use for transformation
   Agrobacterium preparation and Induction:
1. Start 6 mL shaking cultures of LBA4404 Agrobacterium strains harboring rice promoter binary plasmids.
2. Grow the cultures at room temperature for 48 hrs in the rotary shaker.
3. Spin down the cultures at 8'000 rpm at 4° C. and re-suspend bacterial pellets in 10 ml of KCMS media supplemented with 100□M Acetosyringone.
4. Place in the shaker at room temp for 1 hr for induction of Agrobacterium virulence genes.
5. In a sterile hood dilute Agrobacterium cultures 1:3 in KSMS media and transfer diluted cultures into deep petri dishes.
   Inoculation of Plant Material and Staining:
6. In a sterile hood transfer embryogenic callus into diluted Agrobacerium solution and incubate for 30 minutes.
7. In a sterile hood blot callus tissue on sterile filter paper and transfer on MS-AS plates.
8. Co-culture plates in 22° C. growth chamber in the dark for two days.
9. In a sterile hood transfer callus tissue to MS-Tim plates for the tissue recovery (the presence of Timentin will prevent Agrobacterium growth).
10. Incubate tissue on MS-Tim media for two days at 22° C. in the dark.
11. Remove callus tissue from the plates and stain for 48 hrs. in GUS staining solution.
12. De-stain tissue in 70% EtOH for 24 hours.
   Recipies:

KCMS Media (Liquid), pH to 5.5
100 ml/l MS Major Salts, 10 ml/l MS Minor Salts, 5 ml/l MS iron stock, 0.5M K₂HPO₄, 0.1 mg Myo-Inositol,
1.3 μg/ml Thiamine, 0.2 g/ml 2,4-D (1 mg/ml), 0.1 g/ml Kinetin, 3% Sucrose, 100□M Acetosyringone

MS-CIM Media, pH 5.8
MS Basal salt (4.3 g/L), B5 Vitamins (200×) (5 m/L), 2% Sucrose (20 g(L), Proline (500 mg/L), Glutamine (500 mg/L), Casein Hydrolysate (300 mg/L), 2□g/ml 2,4-D, Phytagel (3 g/L)

MS-As Medium, pH 5.8
MS Basal salt (4.3 g/L), B5 Vitamins (200×) (5 m/L), 2% Sucrose (20 g/L), Proline (500 mg/L), Glutamine (500 mg/L), Casein Hydrolysate (300 mg/L), 2□g/ml 2,4-D, Phytagel (3 g/L), 200 □M Acetosyringone

MS-Tim Media, pH 5.8
MS Basal salt (4.3 g/L), B5 Vitamins (200×) (5 m/L), 2% Sucrose (20 g/L), Proline (500 mg/L), Glutamine (500 mg/L), Casein Hydrolysate (300 mg/L), 2□g/ml 2,4-D, Phytagel (3 g/L), 400 mg/l Timentin

Gus Staining Solution, pH 7
0.3M Mannitol; 0.02M EDTA, pH=7.0; 0.04 NaH₂PO₄; 1 mM x-gluc

The binary Promoter::Reporter Plasmids described in Example 19 above can also be used for stable transformation of rice and maize plants according to the protocols provided in Examples 18.1 and 18.2, respectively.

Maize plants stably transformed with the pNOV6070 containing the promoter fragement of SEQ ID NO: 42 shows good expression of the GUS gene in leafs and an intermediate expression in roots.

### Example 21

### Method of Modifying the Gene Frequency

The invention further provides a method of modifying the frequency of a phosphate transporter gene in a plant population, including the steps of: identifying an SSR within a coding region of a phosphate transporter gene; screening a plurality of plants using the SSR as a marker to determine the presence or absence of the phosphate transporter gene in an individual plant; selecting at least one individual plant for breeding based on the presence or absence of the phosphate transporter gene; and breeding at least one plant thus selected to produce a population of plants having a modified frequency of the phosphate transporter gene. The identification of the SSR within the coding region of a phosphate transporter gene can be accomplished based on sequence similarity between the nucleic acid molecules of the invention and the region within the gene of interest flanking the SSR.

### Example 22

### Chromosomal Markers to Identify the Location of a Nucleic Acid Sequence

The sequences of the present invention can also be used for SSR mapping. SSR mapping in rice has been described by Miyao et al. (DNA Res 3:233 (1996)) and Yang et al. (Mol Gen Genet 245:187 (1994)), and in maize by Ahn et al. (Mol Gen Genet 241:483 (1993)). SSR mapping can be achieved using various methods. In one instance, polymorphisms are identified when sequence specific probes flanking an SSR contained within a sequence are made and used in polymerase chain reaction (PCR) assays with template DNA from two or more individuals or, in plants, near isogenic lines. A change in the number of tandem repeats between the SSR-flanking sequence produces differently sized fragments (U.S. Pat. No. 5,766,847). Alternatively, polymorphisms can be identified by using the PCR fragment produced from the SSR-flanking sequence specific primer reaction as a probe against Southern blots representing different individuals (Refseth et al., Electrophoresis 18:1519 (1997)). Rice SSRs can be used to map a molecular marker closely linked to functional gene, as described by Akagi et al. (Genome 39:205 (1996)).

The sequences of the present invention can be used to identify and develop a variety of microsatellite markers, including the SSRs described above, as genetic markers for comparative analysis and mapping of genomes.

Many of the polynucleotides listed in Table 1 contain at least 3 consecutive di-, tri- or tetranucleotide repeat units in their coding region that can potentially be developed into SSR markers. Trinucleotide motifs that can be commonly found in the coding regions of said polynucleotides and easily identified by screening the polynucleotides sequences for said motifs are, for example: CGG (see, for example, SEQ ID NO: 31); GCC (see, for example, SEQ ID NOs: 8, 10 and 40), CGC (see, for example, SEQ ID NOs: 1 and 10), GGC (see, for example, SEQ ID NOs: 11 and 35), etc. Once such a repeat unit has been found, primers can be designed which are complementary to the region flanking the repeat unit and used in any of the methods described below.

Sequences of the present invention can also be used in a variation of the SSR technique known as inter-SSR (ISSR), which uses microsatellite oligonucleotides as primers to amplify genomic segments different from the repeat region itself (Zietkiewicz et al., Genomics 20:176 (1994)). ISSR employs oligonucleotides based on a simple sequence repeat anchored or not at their 5′- or 3′-end by two to four arbitrarily chosen nucleotides, which triggers site-specific annealing and initiates PCR amplification of genomic segments which are flanked by inversely orientated and closely spaced repeat sequences. In one embodiment of the present invention, microsatellite markers as disclosed herein, or substantially similar sequences or allelic variants thereof, may be used to detect the appearance or disappearance of markers indicating genomic instability as described by Leroy et al. (Electron. J Biotechnol, 3(2), at http://www.ejb.org (2000)), where alteration of a fingerprinting pattern indicated loss of a marker corresponding to a part of a gene involved in the regulation of cell proliferation. Microsatellite markers are useful for detecting genomic alterations such as the change observed by Leroy et al. (Electron. J Biotechnol, 3(2), supra (2000)) which appeared to be the consequence of microsatellite instability at the primer binding site or modification of the region between the microsatellites, and illustrated somaclonal variation leading to genomic instability. Consequently, sequences of the present invention are useful for detecting genomic alterations involved in somaclonal variation, which is an important source of new phenotypes.

In addition, because the genomes of closely related species are largely syntenic (that is, they display the same ordering of genes within the genome), these maps can be used to isolate novel alleles from wild relatives of crop species by positional cloning strategies. This shared synteny is very powerful for using genetic maps from one species to map genes in another. For example, a gene mapped in rice provides information for the gene location in maize and wheat.

### Example 23

### Quantitative Trait Linked Breeding

Various types of maps can be used with the sequences of the invention to identify Quantitative Trait Loci (QTLs) for a variety of uses, including marker-assisted breeding. Many important crop traits are quantitative traits and result from the combined interactions of several genes. These genes reside at different loci in the genome, often on different chromosomes, and generally exhibit multiple alleles at each locus. Developing markers, tools, and methods to identify and isolate the QTLs involved in a trait, enables marker-assisted breeding to enhance desirable traits or suppress undesirable traits. The sequences disclosed herein can be used as markers for QTLs to assist marker-assisted breeding. The sequences of the invention can be used to identify QTLs and isolate alleles as described by Li et al. in a study of QTLs involved in resistance to a pathogen of rice. (Li et al., Mol Gen Genet 261:58 (1999)). In addition to isolating QTL alleles in rice, other cereals, and other monocot and dicot crop species, the sequences of the invention can also be used to isolate alleles from the corresponding QTL(s) of wild relatives. Transgenic plants having various combinations of QTL alleles can then be created and the effects of the combinations measured. Once an ideal allele combination has been identified, crop improvement can be accomplished either through biotechnological means or by directed conventional breeding programs. (Flowers et al., J Exp Bot 51:99 (2000); Tanksley and McCouch, Science 277:1063 (1997)).

### Example 24

### Marker-Assisted Breeding

Markers or genes associated with specific desirable or undesirable traits are known and used in marker assisted breeding programs. It is particularly beneficial to be able to screen large numbers of markers and large numbers of candidate parental plants or progeny plants. The methods of the invention allow high volume, multiplex screening for numerous markers from numerous individuals simultaneously.

A multiplex assay is designed providing SSRs specific to each of the markers of interest. The SSRs are linked to different classes of beads. All of the relevant markers may be expressed genes, so RNA or cDNA techniques are appropriate. RNA is extracted from root tissue of 1000 different individual plants and hybridized in parallel reactions with the different classes of beads. Each class of beads is analyzed for each sample using a microfluidics analyzer. For the classes of beads corresponding to qualitative traits, qualitative measures of presence or absence of the target gene are recorded. For the classes of beads corresponding to quantitative traits, quantitative measures of gene activity are recorded. Individuals showing activity of all of the qualitative genes and highest expression levels of the quantitative traits are selected for further breeding steps. In procedures wherein no individuals have desirable results for all the measured genes, individuals having the most desirable, and fewest undesirable, results are selected for further breeding steps. In either case, progeny are screened to further select for homozygotes with high quantitative levels of expression of the quantitative traits.

References

Abel et al., Science, 232:738 (1986).
Aharoni et al., Plant Cell, 5:613 (2000).
Altschul et al. Nucleic Acids Res., 25:3389 (1997).
Altschul et al., J. Mol. Biol., 215:403 (1990).
An et al., EMBO J., 4:277 (1985).
Aoyama et al., Plant Journal, 11:605 (1997).
AtMas, et al, Plant Mol. Biol., 2:335 (1983).
Auch & Reth, Nuclein Acids Research, 18:6743 (1990).
Ballas et al., Nucleic Acids Res., 17:7891 (1989).
Bansal et al., Proc. Natl. Acad. Sci. USA, 89:3654 (1992).
Barkai-Golan et al., Arch. Microbiol., 116:119 (1978).
Barton et al., Plant Physiol., 85:1103 (1987).
Batzer et al., Nucleic Acid Res., 19:5081 (1991).
Beals et al., Plant Cell, 9:1527 (1997).
Bécard, G. and Fortin, J A (1987) New Phytol 108, 211-218.
Belanger et al., Genetics, 129:863 (1991).
Bemal-Lugo and Leopold, Plant Physiol., 98:1207 (1992).
Bevan et al., Nucl. Acids Res., 11:369 (1983).
Bevan et al., Nature, 304:184 (1983).
Bevan, Nucl. Acids Res., 12:8711 (1984).
Bird et al., Plant Molecular Biology, 11:651 (1988).
Bisaro, Homologous Recomb. Gene Silencing Plants, pp. 219-70, Paszkowski & Jerzy (eds.) (1994).
Blackman et al., Plant Physiol., 100:225 (1992).
Blochlinger & Diggelmann, Mol Cell Biol, 4:2929 (1984).
Bol et al., Ann. Rev. Phytopath., 28:113 (1990).
Bouchez et al., EMBO J., 8:4197 (1989).
Bouchez et al., EMBO Journal, 8:4197 (1989).
Bourouis et al., EMBO J., 2:1099 (1983).
Bowler et al., Ann. Rev. Plant Physiol., 43:83 (1992).
Bradford, M. M. (1976) Anal Biochem 72, 248-254
Branson and Guss, Proc. North Central Branch Entomological Society of America (1972).
Broakgert et al., Science, 245:110 (1989).
Brown et al., PNAS USA, 97:262 (2000).
Brundrett, M. C., Piché, Y. and Peterson, R. L. (1984) Can. J. Bot. 62, 2128-2134
Bun-Ya, M., Nishimura, M., Harashima, S., Oshima, Y. (1991) Mol Cell Biol. 11, 3229-3238
Burge, C. and Karlin, S. (1997) J. Mol. Biol. 268, 78-94
Burleigh, S. H. and Harrison, M. J. (1997) Plant Mol Biol. 34, 199-208
Byrne et al. Plant Cell Tissue and Organ Culture, 8:3 (1987).
Callis et al., Genes and Develope., 1:1183 (1987).
Callis et al., J. Biol. Chem., 265:12486 (1990).
Campbell and Gowri, Plant Physiol., 92:1 (1990).
Castrsana et al., EMBO J., 7:1929 (1988).
Chandleret al., Plant Cell, 1:1175 (1989).
Chee et al. Plant Physiol., 91:1212 (1989).
Chee et al., Methods Mol. Biol., 44:101 (1995).
Chrispeels, M. J., Crawford, N. M. and Schroeder, J. I. (1999) Plant Cell 11, 661-676
Christou et al. Proc. Natl. Acad. Sci USA, 86:7500 (1989).
Christou et al., Biotechnology, 9:957 (1991).
Christou et al., Plant Physiol., 87:671 (1988).
Cirillo, V. P. (1989) Methods Enzymol. 174, 617-622
Coe et al., In: Corn and Corn Improvement, Sprague et al. (eds.) pp. 81-258 (1988).
Cordero et al., Plant J., 6:141 (1994).
Corpet et al. Nucleic Acids Res., 16:10881 (1988).
Coxson et al., Biotropica, 24:121 (1992).
Crameri et al., Nature Biotech., 15:436 (1997).
Crameri et al., Nature, 391:288 (1998).
Crossway et al., BioTechniques, 4:320 (1986).
Cserzo, M., Wallin, E., Simon, I., von Heijne G. and Elofsson A. (1997) Prot. Eng. 10, 673-676
Cuozzo et al., Bio/Technolog, 6:549 (1988).
Cutler et al., J. Plant Physiol., 135:351 (1989).
Czako et al., Mol. Gen. Genet., 235:33 (1992).
Czapla and Lang, J. Econ. Entomol., 83:2480 (1990).
Daram, P., et al. (1998) Planta 206, 225-233
Datta et al., Bio/Technology, 8:736 (1990).
Davies et al., Plant Physiol., 93:588 (1990).
Dayhoff et al., Atlas of Protein Sequence and Structure, Natl. Biomed. Res. Found., Washington, C. D. (1978).
De Blaere et al., Meth. Enzymol., 143:277 (1987).
De Block et al. Plant Physiol., 91:694 (1989).
De Block et al., EMBO Journal, 6:2513 (1987).
Della-Cioppa et al., Plant Physiology, 84:965-968 (1987).
Dellaporta et al., in Chromosome Structure and Function, Plenum Press, 263-282 (1988).
Dennis et al., Nucleic Acids Res., 12:3983 (1984).
Depicker et al., Plant Cell Reports, 7:63 (1988).
DeRisi et al., Science, 278:680 (1997).
Desprez et al., Plant J., 14:643 (1998).
Diekman & Fischer, EMBO, 7:3315 (1988).
Duggan et al., Nat. Genet., 21:10 (1999).
Dunn et al., Can. J. Plant Sci., 61:583 (1981).
Dure et al., Plant Mol. Biol., 12:475 (1989).
Eisen et al., PNAS USA, 95:14863 (1998).
Elble, R. (1992) Biotechnigues 13, 18-20
Ellis et al., EMBO Journal, 6:3203 (1987).
Elroy-Stein et al., Proc. Natl. Acad. Sci. U.S.A., 86:6126 (1989).
English et al., Plant Cell, 8:179 (1996).
Erdmann et al., J. Gen. Microbiol., 138:363 (1992).
Everett et al., Bio/Technology, 5:1201(1987).
Fitzpatrick, Gen. Engineering News, 22:7 (1993).
Franken et al., EMBO J., 10:2605 (1991).
Fromm et al., Nature (London), 319:791 (1986).
Fromm et al., Bio/Technoloy, 8:833 (1990).
Gallie et al., Nucleic Acids Res., 15:3257 (1987).
Gallie et al., The Plant Cell, 1:301 (1989).
Gan et al., Science, 270:1986 (1995).
Gatehouse et al., J. Sci. Food Agric., 35:373 (1984).
Gatz, Current Opinion in Biotechnology, 7:168 (1996).
Gatz, Annu. Rev. Plant Physiol. Plant Mol. Biol., 48:89 (1997).
Gelfand, eds., PCR Strategies Academic Press, New York (1995).
Gelvin et al., Plant Molecular Biology Manual, (1990).
Giege et al., Plant J., 15:721 (1998).
Giovanetti, M. and Mosse, B. (1980) New Phytol. 84: 489-500
Goff, S. A., et al. (2002) Science . . .
Gordon-Kamm et al., Plant Cell, 2:603 (1990).
Goring et al, PNAS, 88:1770 (1991).
Graham et al., Biochem. Biophys. Res. Comm., 101:1164 (1981).
Graham et al., J. Biol. Chem., 260:6555 (1985).
Graham et al., J. Biol. Chem., 260:6561 (1985).
Gritz et al., Gene, 25:179 (1983).
Gruber, et al., Vectors for Plant Transformation, in: Methods in Plant Molecular Biology & Biotechnology” in Glich et al., (Eds. pp. 89-119, CRC Press, 1993).
Guerineau et al., Mol. Gen. Genet., 262:141 (1991).
Guerrero et al., Plant Mol. Biol., 15:11 (1990).
Gupta et al., PNAS, 90:1629 (1993).
Haines and Higgins (eds.), Nucleic Acid Hybridization, IRL Press, Oxford, U.K.
Hammock et al., Nature, 344:458 (1990).
Harrison, M. J. (1999) Annu. Rev. Plant Physiol. Plant Mol. Biol. 50: 361-389
Hemenway et al., EMBO Journal, 7:1273 (1988).
Henikoff & Henikoff, Proc. Natl. Acad. Sci. USA, 89:10915 (1989).
Hiei et al., Plant J., 6:271 (1994).
Higgins et al., CABIOS, 5:151 (1989).
Higgins et al., Gene, 73:237 (1988).
Hilder et al., Nature, 330:160 (1987).
Hinchee et al. Bio/Technology 6:915 (1988).
Hoekema, In: The Binary Plant Vector System. Offset-drukkerij Kanters B. V.; Alblasserdam (1985).
Hofmann, K. and Stoffel, W. (1993) Biol. Chem. Hoppe-Seyler 374, 166
Huang et al., CABIOS, 8:155 (1992).
Hudspeth & Grula, Plant Molec. Biol., 12, 579 (1989).
Hughes et al., J. Mol. Biol., 296:1205 (2000).
Ikeda et al., J. Bacteriol., 169:5612 (1987).
Icuta et al., Biotech., 8:241 (1990).
Ingelbrecht et al., Plant Cell, 1:671 (1989).
Innis et al., PCR Protocols: A Guide to Methods and Applications, Academic Press, Inc., San Diego, Calif. (1990).
Innis and Gelfand, eds., PCR Methods Manual (Academic Press, New York) (1999).
Innis et al., eds., PCR Protocols: A Guide to Methods and Applications (Academic Press, New York (1995).
Jauneau A., Quentin M. and Driouich A. (1997) Protoplasma 198, 9-19.
Jefferson et al, EMBO J, 6: 3901-3907 (1987).
Jobling et al., Nature, 325:622 (1987).
John et al., Proc. Natl. Acad. Sci. USA, 89:5769 (1992).
Johnson et al., PNAS USA, 86:9871 (1989)
Joshi et al., Nucleic Acid Res., 15:9627 (1987).
Kaasen et al., J. Bacteriol., 174:889 (1992).
Kagaya et al., Nucleic Acids Res., 27:470 (1999).
Karlin and Altschul, Proc. Natl. Acad Sci. USA, 87:2264 (1990).
Karlin and Altschul, Proc. Natl. Acad. Sci. USA, 90:5873 (1993).
Karsten et al., Botanica Marina, 35:11 (1992).
Katz et al., J. Gen. Microbiol., 129:2703 (1983).
Kehoe et al., Trends Plant Sci., 4:38 (1999).
Kelleret al., EMBO Journal, 8:1309 (1989).
Keller et al., Genes Dev., 3:1639 (1989).
Klein et al., Nature, 327:70 (1987).
Klein et al., Bio/Technology, 6:559 (1988).
Klein et al., Plant Physiol., 91:440 (1988).
Klein et al., Proc. Natl. Acad. Sci. USA, 85:4305 (1988).
Knauf, et al., Genetic Analysis of Host Range Expression by Agrobacterium In: Molecular Genetics of the Bacteria-Plant Interaction, Puhler, A. ed., Springer-Verlag, New York, 1983.
Koehl P. and Delarue M., Curr. Opin. Struct. Biol., 6:222 (1996).
Kohler et al., Plant Mol. Biol., 29:1293 (1995).
Koster and Leopold, Plant Physiol., 88:829 (1988).
Koziel et al., Biotechnology, 11:194 (1993).
Kridl et al., Seed Science Research, 1:209 (1991).
Kriz et al., Mol. Gen. Genet., 207:90 (1987).
Krogh, A., et al (2001) J. Mol. Biol. 305, 567-580
Kunkel et al., Methods in Enzymol., 154:367 (1987).
Kunkel, Proc. Natl. Acad. Sci. USA, 82:488 (1985).
Lam etal., Plant Cell, 1:1147 (1989).
Landolt, Biosystematic Investigation on the Family of Duckweeds: The family of Lemnaceae—A Monograph Study. Geobatanischen Institut ETH, Stiftung Rubel, Zurich (1986).
Langridge et al., Proc. Natl Acad. Sci. U.S.A., 86:3219 (1989).
Langridge et al., Cell, 34:1015 (1983).
Lashkari et al., PNAS USA, 94:8945 (1997).
Laufs et al., PNAS, 87:7752 (1990).
Lawton et al., Mol. Cell Biol., 7:335 (1987).
Lee and Saier, J. Bacteriol., 153 (1982).
Leggewie G., et aö (1997) Plant Cell 9:381-92
Lesyng B. and McCammon J A, Pharmocol. Ther., 60:149 (1993).
Levings, Science, 250:942 (1990).
Lindsey et al., Transgenic Research, 2:3347 (1993).
Lindstrom et al., Der. Genet., 11:160 (1990).
Liu H., et al (1998) Mol. Plant Microbe Interact. 11,14-22
Lockhart et al., Nat. Biotechnol, 14:1649 (1996).
Lockhart and Winzeler, Nature, 405:827 (2000).
Lommel et al., Virology, 181:382 (1991).
Loornis et al., J. Expt. Zool., 252:9 (1989).
Lorz et al., Mol. Gen. Genet., 199:178 (1985).
Lukashin, A. V. and Borodovsky, M. (1998) Nucleic Acids Res 26,1107-1115
Lyznik et al., Nucleic Acids Res., 21:969 (1993).
Ma et al., Nature, 334 :631 (1988).
Macejak et al., Nature, 353:90 (1991).
Maki et al., Methods in Plant Molecular Biology & Biotechnology, Glich et al., 67-88 CRC Press, (1993).
Maleck et al., Nat. Genet., 26:403 (2000).
Mansson et al., Gen. Genet., 200:356 (1985).
Mariani et al, Nature, 347:737 (1990).
Martinez et al., J. Mol. Biol., 208:551 (1989).
McBride et al., Plant Molecular Biology, 14:266 (1990).
McBride et al., PNAS USA, 91:7301 (1994).
McCabe et al., Bio/Technology, 6:923 (1988).
McElroy et al., Mol. Gen. Genet., 231:150 (1991).
Meinkoth and Wahl, Anal. Biochem., 138:267 (1984).
Messing and Vierra, Gene, 19:259 (1982).
Michael et al., J. Mol. Biol., 26 :585 (1990).
Mogen et al., Plant Cell, 2:1261 (1990).
Moore et al., J. Mol. Biol., 272:336 (1997).
Muchhal, U. S., et al (1996) Proc Natl Acad Sci 93, 10519-10523
Mundy and Chua, EMBO J., 7:2279 (1988).
Munroe et al., Gene, 91:151 (1990).
Murakami et al., Mol. Gen. Genet., 205:42 (1986).
Murata et al., FEBS Lett., 296:187 (1992).
Murdock et al., Phytocheistry, 29:85 (1990).
Murray et al., Nucleic Acids Res., 17:477 (1989).
Myers and Miller, CABIOS, 4:11 (1988).
Napoli et al., Plant Cell, 2:279 (1990).
Narasimhulu et al, Plant Cell, 8: 873-886, (1996).
Needleman and Wunsch, J. Mol. Biol., 48:443-453 (1970).
Newman et al., Plant Physiol., 106:1241 (1994).
Niedz et al., Plant Cell Reports, 14:403 (1995).
Odell et al., Mol. Gen. Genet., 113:369 (1990).
Odell et al., Homologous Recomb. Gene Silencing Plants, 219-70, Paszkowski & Jerzy (eds) (1994).
Odell et al., Nature, 313:810 (1985).
Ohtsuka et al., J. Biol. Chem., 260:2605 (1985).
Ow et al., Science, 234:856 (1986).
Pacciotti et al., Bio/Technolog, 3:241 (1985).
Park et al., J. Plant Biol., 38:365 (1985).
Paszkowski et al., EMBO J., 3:2717 (1984).
Pear et al., Plant Molecular Biology, 13:639 (1989).
Pearson and Lipman, Proc. Natl. Acad. Sci., 85:2444 (1988).
Pearson et al., Meth. Mol. Biol., 24:307 (1994).
Pearson, J. N. and Jakobsen, I. (1993). New Phytol., 124,489-494
Perlak et al., Proc. Natl. Acad. Sci. USA, 88:3324 (1991).
Phillips et al., In Corn & Corn Improvement, 3rd Edition 10 Sprague et al. (Eds. pp. 345-387)(1988).
Phi-Van et al., Mol. Cell. Biol., 10:2302 (1990).
Piatkowski et al., Plant Physiol., 94:1682 (1990).
Potrykus et al., Mol. Gen. Genet., 199:183 (1985).
Potrykus, Trends Biotech., 7:269 (1989).
Poulsen et al., Mol. Gen. Genet., 205:193 (1986).
Prasher et al., Biochem. Biophys. Res. Comm., 126:1259 (1985).
Proudfoot, Cell, 64:671 (1991).
Quigley et al., J. Mol. Evol., 29:412 (1989).
Ralston et al., Genetics, 119:185 (1988).
Raghothama, K. G. (2000) Curr. Opin. Plant Biol. 3,182-187
Rausch, C., et al. (2001) Nature 414, 462-470
Reed et al., J. Gen. Microbiol., 130:1 (1984).
Reina et al., Nucleic Acids Res., 18:6425 (1990).
Reina et al., Nucleic Acids Res., 18:7449 (1990).
Remy, W., Taylor, T. N., Hass, H. and Kerp, H. (1994) Proc Natl Acad Sci 91, 11841-11843
Reymond et al., Plant Cell, 12:707 (2000).
Richmond et al., Curr Opin Plant Biol., 3:108 (2000).
Riggs et al., Proc. Natl. Acad. Sci. USA, 83:5602 (1986).
Rosewarne, G. M., et al. (1999) New Phytol., 144, 507-516
Rossi et al., Biophys. J., 80:480 (2001).
Rossolini et al., Mol. Cell. Probes, 8:91 (1994).
Rothstein et al., Gene, 53:153 (1987).
Ruiz, Plant Cell, 10:937 (1998).
Safak et al., Mol. Cell Biol., 19:2712 (1999).
Salamov, A. A. and Solovyev, V. V. (1999) Genome Res. 10, 516-22
Sambrook et al., Molecular Cloning: A Laboratory Manual (2d ed., Cold Spring Harbor Laboratory Press, Plainview, N.Y.) (1989).
Sanfacon et al., Genes Dev., 5:141 (1991).
Sanford et al., Particulate Science and Technology, 5:27 (1987).
Schachtman, D. P., Reid, R. J. and Ayling, S. M. (1998) Cell. Plant Physiol. 116,447-453
Schaffer et al., Curr Opin Biotechnol., 11:162 (2000).
Schena et al., Science, 270:467 (1995).
Schenk et al., PNAS USA, 97:11655 (2000).
Schmidhauser and Helinski, J. Bacteriol., 164:446 (1985).
Schwob et al., Plant J., 4:423 (1993).
Shagan et al., Plant Physiol., 101:1397 (1993).
Shapiro, Mobile Genetic Elements, Academic Press, N.Y. (1983).
Shimamoto et al., Nature, 338:274 (1989).
Simon, l. Bousquet, J., Levesque, R. C. and Lalonde, M. (1993) Nature 363, 67-69
Simpson, Plant Mol. Biol., 19:699 (1985).
Skriver and Mundy, Plant Cell, 2:503 (1990).
Skuzeski et al., Plant Molec. Biol. 15: 65-79 (1990).
Slater et al., Plant Mol. Biol., 5:137 (1985).
Smith et al., Adv. Appl. Math., 2:482 (1981).
Smith et al., Mol. Gen. Genet., 224:447 (1990).
Smith et al., Planta, 168:94 (1986).
Smith, F. W., Ealing, P. M., Dong, B. and Delhaize, E. (1997) Plant J. 11, 83-92
Smith, F. W., Rae, A. L. and Hawkesford, M. J. (2000) Biochim. Biophys. Acta 1465, 236-45
Smith, S. E. and Read, D. J., eds. (1997) Mycorrhizal Symbiosis. San Diego, Calif.: Academic
Southern et al., Nature Genet., 21:5-9 (1999).
Spencer et al., Theor. Appl. Genet, 79:625 (1990).
Stalker et al., Science, 242:419 (1988).
Staub et al., EMBO J., 12:601 (1993).
Staub et al., Plant Cell, 4:39 (1992).
Steifel et al., The Plant Cell, 2:785 (1990).
Stemmer, Nature, 370:389 (1994).
Stemmer, Proc. Natl. Acad. Sci. USA, 91:10747 (1994).
Stief et al., Nature, 341:343 (1989).
Stouggard, The Plant Journal, 3:755 (1993).
Sukhapinda et al., Plant Mol. Biol., 8:209 (1987).
Sullivan et al., Mol. Gen. Genet., 215:431 (1989).
Surles et al., Protein Sci., 3:198 (1994).
Sutcliffe, PNAS USA, 75:3737 (1978).
Svab et al., Proc. Natl. Acad. Sci. USA, 87:8526 (1990).
Svab et al., Proc. Natl. Acad. Sci. USA, 90:913 (1993).
Tamayo et al., PNAS USA, 96:2907 (1999).
Tarczynski et al., PNAS USA, 89:2600 (1992).
Thillet et al., J. Biol. Chem., 263:12500 (1988).
Thompson, J. D., Higgins, D. G. and Gibson, T. J. (1994) Nucleic Acids Res. 22:4673-4680
Thompson et al., EMBO J, 6:2519 (1987).
Tijssen, Laboratory Techniques in Biochemistry and Molecular Biology-Hybridization with Nucleic Acid Probes, Elsevier, N.Y. (1993).
Tomes et al., Plant Cell, Tissue and Organ Culture: Fundamental Methods, Springer Verlag, Berlin (1995).
Tomic et al., NAR, 12:1656 (1990).
Tremousaygue et al., Plant J., 20:553 (1999).
Turner et al., Molecular Biotechnology, 3:225 (1995).
Tusnady, G. E. and Simon, I. (1998) J. Mol. Biol. 283, 489-506
Twell et al., Plant Physiol., 91:1270 (1989).
Ugaki et al., Nucl. Acids Res., 19:371 (1991).
Ulmasov et al., Plant Mol. Biol., 35:417 (1997).
Upender et al., Biotechniques, 18:29 (1995).
Vaeck et al., Nature, 328:33 (1989).
van der Krol et al., Plant Cell, 2:291 (1990).
vanTunen et al., EMBO J., 7:1257 (1988).
Vasil et al., Biotechnology, 11:1553 (1993).
Vasil et al., Mol. Microbiol., 3:371 (1989).
Vasil et al., Plant Physiol., 91:1575 (1989).
Vernon and Bohnert, EMBO J., 11:2077 (1992).
Vodkin, Prog. Clin. Biol. Res., 138:87 (1983).
Vogel et al., EMBO J., 11:157 (1992).
Walker and Gaastra, eds., Techniques in Molecular Biology, MacMillan Publishing Company, New York (1983).
Wandelt et al., Nucleic Acids Res., 17:2354 (1989).
Wang et al., Mol. Cell. Biol., 12:3399 (1992).
Wang, J., Jiang, J. and Oard, J. H. (2000) Plant Sci. 156, 201-211
Watennan, M. S. Introduction to Computational Biology: Maps, sequences and genomes. Chapman & Hall. London (1995).
Watson et al., Corn: Chemistry and Technology (1987).
Watrud et al., in Engineered Organisms and the Environment (1985).
Weeks et al., Plant Physiol., 102:1077 (1993).
Weissinger et al., Annual Rev. Genet., 22:421 (1988).
Wenzler et al., Plant Mol. Biol., 13:347 (1989).
White et al, Nucl Acids Res, 18, 1062 (1990).
Wolter et al., EMBO Journal, 11:4685 (1992).
Wyn-Jones and Storey, Physiology and Biochemistry of Drought Resistance in Plants, Paleg et al. (eds.), pp. 171-204 (1981).
Xiang and Guerra, Plant Physiol., 102:287 (1993).
Yamaguchi-Shinozaki et al., Plant Cell Physiol., 33:217 (1992).
Yamamoto et al., Nucleic Acids Res., 18:7449 (1990).
Yanagisawa and Schmidt, Plant J., 17:209 (1999).
Yanagisawa et al., Plant J., 21:281-288 (2000).
Yuan et al., Plant J., 15:821 (1998).
Zhang et al., Proc. Natl. Acad. Sci. USA, 94:4504 (1997).
Zhu et al., Nat. Biotechnol., 18:555-558 (2000).
Zhu et al., Plant Physiol., 124:1472 (2000).
Zhu et al., Proc. Natl. Acad. Sci. USA, 96:8768-8773 (1999).
Zukowsky et al., PNAS USA, 80:1101 (1983).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. An isolated nucleic acid molecule comprising a nucleotide sequence encoding a polypeptide that mediates phosphate uptake into the plant cell in the presence of a mycorrhizal fungus, but not under conditions of phosphate deficiency or phosphate sufficiency, and, optionally, a regulatory region, which induces a mycorrhiza dependent and phosphate independent transcription of the coding region.

2. The nucleic acid molecule of claim 1, wherein the mycorrhizal fungus is an arbusular mycorrhizal fungus.

3. The nucleic acid molecule of claim 1, which comprises a nucleotide sequence substantially similar to a nucleotide sequence encoding a polypeptide as given in SEQ ID NO: 24.

4. The nucleic acid molecule of claim 3 comprising a nucleotide sequence encoding a polypeptide which polypeptide has at least between 70%, and 99% amino acid sequence identity to the polypeptide of SEQ ID NO: 24, or a partial-length polypeptide having substantially the same activity as the full-length polypeptide, e.g., at least 50%, more preferably at least 80%, even more preferably at least 90% to 95% the activity of the full-length polypeptide.

5. The nucleic acid molecule of claim 4 comprising a nucleotide sequence encoding a polypeptide which polypeptide has at least 90% amino acid sequence identity to the polypeptide of SEQ ID NO: 24, or a partial-length polypeptide having substantially the same activity as the full-length polypeptide, e.g., at least 50%, more preferably at least 80%, even more preferably at least 90% to 95% the activity of the full-length polypeptide.

6. The nucleic acid molecule of claim 3 comprising a nucleotide sequence encoding a polypeptide which polypeptide is immunologically reactive with antibodies raised against a polypeptide of SEQ ID NO: 24; or a partial-length polypeptide having substantially the same activity as the full-length polypeptide, e.g., at least 50%, more preferably at least 80%, even more preferably at least 90% to 95% the activity of the full-length polypeptide.

7. The nucleic acid molecule of claim 1 comprising a nucleotide sequence
a) as given in SEQ ID NO: 11, or a part thereof, which encodes a partial-length polypeptide that still has substantially the same activity as the full-length polypeptide, e.g., at least 50%, more preferably at least 80%, even more preferably at least 90% to 95% the activity of the full-length polypeptide;
b) having substantial similarity to (a);
c) capable of hybridizing to (a) or the complement thereof;
d) capable of hybridizing to a nucleic acid comprising 50 to 200 or more consecutive nucleotides of a nucleotide sequence given in SEQ ID NO: 11 or the complement thereof;
e) complementary to (a), (b) or (c); or
f) which is the reverse complement of (a), (b) or (c).

8. The nucleic acid molecule of claim 1 comprising a nucleotide sequence encoding a polypeptide which polypeptide is substantially similar to a polypeptide encoded by a nucleotide sequence comprising a promoter sequence as given in SEQ ID NO: 39.

9. The nucleic acid molecule of claim 1 comprising a nucleotide sequence which is substantially similar to a nucleotide sequence comprising a promoter sequence as given in SEQ ID NO: 39.

10. The nucleic acid molecule of claim 1, wherein the nucleotide sequence encoding a polypeptide which mediates phosphate uptake into the plant cell in the presence of a mycorrhizal fungus, but not under conditions of phosphate deficiency or phosphate sufficiency is preferentially expressed in plant roots.

11. An isolated nucleic acid molecule comprising a nucleotide sequence encoding a polypeptide that mediates phosphate uptake into the plant cell in response to phosphate availability, that is under conditions of
(a) phosphate deficiency, or;
(b) phosphate sufficiency perferentially in a specific plant tissue, i.e., in roots, green tissue- (leaf- and stem-) or flowers, and, optionally, a regulatory region, which induces a phosphate-dependent transcription of the coding region.

12. The isolated nucleic acid molecule of claim 11 comprising a nucleotide sequence which has at least between 70% and 99% nucleotide sequence identity to a nucleotide sequence encoding a polypeptide as given in SEQ ID NO: 14 to 23 and 25 to 26, 28, 104 and 106, or a partial-length polypeptide having substantially the same activity as the full-length polypeptide, e.g., at least 50%, more preferably at least 80%, even more preferably at least 90% to 95% the activity of the full-length polypeptide.

13. The isolated nucleic acid molecule of claim 11 comprising a nucleotide sequence which has at least 90% nucleotide sequence identity to a nucleotide sequence encoding a polypeptide as given in SEQ ID NO: 14 to 23 and 25 to 26, 28, 104 and 106, or a partial-length polypeptide having substantially the same activity as the full-length polypeptide, e.g., at least 50%, more preferably at least 80%, even more preferably at least 90% to 95% the activity of the full-length polypeptide.

14. The isolated nucleic acid molecule of claim 11 comprising a nucleotide sequence encoding a polypeptide which polypeptide has at least between 70% and 99% amino acid sequence identity to a polypeptide encoded by an Oryza gene as given in any one of the sequences selected from the group consisting of SEQ ID NOs: 1 to 10 and 12 to 13, 27, 103 and 105, or a partial-length polypeptide having substantially the same activity as the full-length polypeptide, e.g., at least 50%, more preferably at least 80%, even more preferably at least 90% to 95% the activity of the full-length polypeptide.

15. The nucleic acid molecule of claim 11 comprising a nucleotide sequence encoding a polypeptide which polypeptide specifically binds to an antibody that has been generated against a polypeptide of SEQ ID NO: 14 to 23 and 25 to 26, 28, 104 and 106, or a partial-length polypeptide having substantially the same activity as the full-length polypeptide, e.g., at least 50%, more preferably at least 80%, even more preferably at least 90% to 95% the activity of the full-length polypeptide.

16. The nucleic acid molecule of claim 11 comprising a nucleotide sequence
(a) as given in SEQ ID NO: 1 to 10, 12 to 13, 27, 103 and 105, or a part thereof, which still encodes a partial-length polypeptide having substantially the same activity as the full-length polypeptide, e.g., at least 50%, more preferably at least 80%, even more preferably at least 90% to 95% the activity of the full-length polypeptide;
(b) having substantial similarity to (a);
(c) capable of hybridizing to (a) or the complement thereof;
(d) capable of hybridizing to a nucleic acid comprising 50 to 200 or more consecutive nucleotides of a nucleotide sequence given in SEQ ID NO: 1 to 10, 12 to 13, 27, 103 and 105 or the complement thereof;
(e) complementary to (a), (b) or (c); or
(f) which is the reverse complement of (a), (b) or (c).

17. The nucleic acid molecule of claim 11 comprising a nucleotide sequence encoding a polypeptide which polypeptide is substantially similar to a polypeptide encoded by a nucleotide sequence comprising a promoter sequence as given in SEQ ID NO: 29 to 38 and 40 to 42.

18. An isolated nucleic acid molecule comprising a plant nucleotide sequence that induces transcription of a linked nucleic acid segment in a plant or plant cell, e.g., a linked nucleic acid molecule comprising an open reading frame for or encoding a structural or regulatory gene, in the presence of a mycorrhizal fungus such as, for example, an arbuscular mycorrhizal fungus, but not under conditions of phosphate deficiency or phosphate sufficiency.

19. The nucleic acid molecule of claim 18 comprising a plant nucleotide sequence that induces transcription of a linked nucleic acid segment in a plant or plant cell, which nucleotide sequence is obtained or obtainable from plant genomic DNA comprising a gene having an open reading frame (ORF) encoding a polypeptide which has at least between 70%, and 99% amino acid sequence identity, to a polypeptide encoded by an Oryza, e.g., Oryza sativa, gene comprising a nucleotide sequence as given in SEQ ID NO: 11.

20. The nucleic acid molecule of claim 18 comprising a plant nucleotide sequence which is the promoter sequence for a gene comprising an ORF that has at least between 70% to 99% nucleic acid sequence identity, to an Oryza gene comprising the sequence as given in SEQ ID NO: 11 and a fragment thereof which has substantially the same promoter activity as the corresponding promoter listed in SEQ ID NO: 39.

21. The nucleic acid molecule of claim 18 comprising a nucleotide sequence
a) as given in SEQ ID NO: 39;
b) having substantial similarity to (a);
c) capable of hybridizing to (a) ) or the complement thereof; or
d) capable of hybridizing to a nucleic acid comprising 50 to 200 or more consecutive nucleotides of a nucleotide sequence given in SEQ ID NO: 39 or the complement thereof.

22. An isolated nucleic acid molecule comprising a plant nucleotide sequence that induces transcription of a linked nucleic acid segment in a plant or plant cell, e.g., a linked nucleic acid molecule comprising an open reading frame for or encoding a structural or regulatory gene, in response to phosphate availability, that is under conditions of
a) phosphate deficiency, or;
b) phosphate sufficiency.

23. The nucleic acid molecule of claim 20 comprising a plant nucleotide sequence that induces transcription of a linked nucleic acid segment in a plant or plant cell, which nucleotide sequence is obtained or obtainable from plant genomic DNA comprising a gene having an open reading frame (ORF) encoding a polypeptide which is substantially similar, and preferably has at least between 70%, and 99% amino acid sequence identity, to a polypeptide encoded by an Oryza, e.g., Oryza sativa, gene comprising a nucleotide sequence as given in SEQ ID NOs: 1 to 10, 12 to 13, 27, 103 and 105.

24. The nucleic acid molecule of claim 21 comprising a plant nucleotide sequence which is the promoter sequence for a gene comprising an ORF that is substantially similar, and preferably has at least between 70% to 99% nucleic acid sequence identity, to an Oryza gene comprising the sequence as given in SEQ ID NO: 1 to 10, 12 to 13, 27, 103 and 105 and a fragment thereof which has substantially the same promoter activity as the corresponding promoter listed in SEQ ID NO: 29 to 38 and 40 to 42.

25. The nucleic acid molecule of claim 22 comprising a nucleotide sequence
(a) as given in SEQ ID NO: 29 to 38 and 40 to 42;
(b) having substantial similarity to (a);
(c) capable of hybridizing to (a) or the complement thereof;
(d) capable of hybridizing to a nucleic acid comprising 50 to 200 or more consecutive nucleotides of a nucleotide sequence given in SEQ ID NO: 29 to 38 and 40 to 42 or the complement thereof;
(e) complementary to (a), (b) or (c); or
(f) which is the reverse complement of (a), (b) or (c).

26. A protein which is preferentially expressed in specific plant tissues, i.e., plant root, flower, and green tissue- (leaf- and stem-) tissue and which is substantially similar to the proteins provided in SEQ ID NOs: 14 to 26 and 28 and 104.

27. A protein substantially similar to the proteins provided in SEQ ID NO: 24, which is highly transcribed in plant roots during symbiotic interaction with a mycorrhizal fungus such as, for example, an arbuscular mycorrhizal fungus.

28. A protein substantially similar to the proteins provided in SEQ ID NO: 15, which is expressed preferentially in roots and upregulated by phosphate starvation, but is downregulated by mycorrhizal fungi.

29. A protein substantially similar to the proteins provided in SEQ ID NO: 20, which is expressed preferentially in flowers.

30. A protein substantially similar to the proteins provided in SEQ ID NO: 28, which is expressed preferentially in green tissue- (leaf- and stem).

31. A protein according to claim 26 wherein said protein has at least 90% amino acid sequence identity to the polypeptide of SEQ ID NOs: 14-26 and 28 and 104, respectively.

32. A recombinant vector molecule comprising an isolated nucleic acid molecule comprising a plant nucleotide sequence according to claim 18 operably linked to an expressible gene of interest

33. A recombinant vector molecule that contains an isolated nucleic acid molecule comprising a plant nucleotide sequence according to claim 22 operably linked to an expressible gene of interest.

34. A recombinant vector molecule according to claim 32, wherein the gene of interest is a gene encoding a polypeptide that mediates phosphte transport in a plant.

35. A recombinant vector molecule comprising a nucleic acid molecule according to claim 1.

36. A host cell comprising a vector of claim 32.

37. A host cell of claim 36 wherein said host cell is a bacterial cell, a yeast cell, an animal cell or a plant cell.

38. The host cell of claim 37, wherein said plant cell is from a cereal.

39. A plant comprising a host cell of claim 36.

40. A plant comprising a recombinant nucleic acid construct comprising a polynucleotide of claim 25.

41. The plant of claim 40, wherein said monocot is a cereal.

42. The plant of claim 39, wherein said plant is selected from the group consisting of maize, soybean, barley, alfalfa, sunflower, canola, tomato, banana, cotton, peanut, sorghum, tobacco, sugarbeet, wheat, and rice.

43. A method of modulating phosphate-uptake in plants, comprising functionally integrating an isolated nucleic acid molecule according to claim 1 comprising a nucleic acid fragment encoding a polypeptide that mediates phosphate uptake into the plant cell, under the transcriptional control of regulatory sequences required for expression in plants, into a cell, group of cells, tissue or organ of a plant.

44. A method of modulating phosphate-uptake in plants, comprising:
a) functionally integrating
i. an isolated nucleic acid molecule according to claim 1 comprising a nucleic acid fragment encoding a phosphate transporter protein, or a portion thereof in an anti-sense orientation; or
ii. an dsRNAi construct comprising an isolated nucleic acid molecule according to claim 1, or a portion thereof in both a sense and an anti-sense orientation, which, optionally, may be separated by a spacer region;
under the transcriptional control of regulatory sequences required for expression in plants, into a cell, group of cells, tissue or organ of a plant; and
b) expressing the constructs as provided in a) and b) above in a cell, group of cells, a tissue or organ of a plant to produce a RNA transcript capable of suppressing the expression of protein from an endogenous gene or a transgene encoding a polypeptide or protein that mediates phosphate uptake into the plant cell, thereby downregulating or knocking out the activity of the endogenous polypeptide or protein.

45. A method according to claim 44, wherein the regulatory sequences include a promoter sequence which specifically induce transcription in response to symbiotic interactions with a fungus.

46. A method according to claim 45, wherein the symbiotically regulated promoter sequence is a promoter sequence according to claim 18.

47. A method according to claim 44, wherein the regulatory sequences include a promoter sequence which specifically induce transcription in response to phosphate availability that is
(a) phosphate deficiency, or;
(b) phosphate sufficiency

48. A method according to claim 47, wherein the phosphate regulated promoter sequence is a promoter sequence according to claim 22.

49. A method according to claim 44, wherein the regulatory sequences include a promoter sequence which promoter sequence is from a constitutive promoter.

50. A method according to claim 44, wherein the cell, group of cells, tissue or organ is part of a plant root.

51. A method according to claim 50, wherein the cell, group of cells, tissue or organ is part of a plant root, flower, and green tissue- (leaf- and stem-) tissue, respectively.

52. A method according to claim 44, wherein the nucleic acid fragment encoding a polypeptide that mediates phosphate uptake into the plant cell is from a nucleic acid molecule according to any one of claim 1.

53. A method according to claim 44, wherein the nucleic acid fragment encoding a polypeptide that mediates phosphate uptake into the plant cell is from a nucleic acid molecule according to any one of claim 11.

54. A method of identifying or isolating polynucleotide sequences that are orthologous to a nucleic acid molecule according to claim 1 comprising a nucleic acid fragment encoding a polypeptide that mediates phosphate uptake into the plant cell, from the genome of another plant, wherein all or a portion of a particular nucleic acid sequence according to claim 1 is used as a probe that selectively hybridizes to gene sequences present in a population of cloned genomic DNA fragments or cDNA fragments from a chosen source organism.

55. A method according to claim 58, wherein the polynucleotide sequences are identified by hybridization screening of plated DNA libraries and amplification by PCR using oligonucleotide primers.

56. A method to identify a nucleic acid molecule encoding a polypeptide that mediates phosphate uptake into the plant cell, the expression of which is altered in root comprising
a) contacting a plurality of isolated nucleic acid samples comprising all or a portion of a particular nucleic acid sequence according to anyone claim 1 on a solid substrate with a probe comprising plant nucleic acid corresponding to RNA isolated from root so as to form a complex, wherein each sample comprises a plurality of oligonucleotides corresponding to at least a portion of one plant gene; and
b) contacting a second plurality of isolated nucleic acid samples on a solid substrate with a second probe comprising plant nucleic acid corresponding to RNA that is not from root;
c) comparing complex formation in a) with complex formation in b) so as to identify which samples correspond to genes that are expressed in root.

57. A method for detecting the presence of a polynucleotide according to claim 1 encoding a polypeptide that mediates phosphate uptake into the plant cell and/or a polynucleotide according to any one of claim 18 representing promoter sequences, or a fragment or a variant thereof, or a complementary sequence thereto in a sample, the method including the following steps of:
(a) bringing into contact a nucleotide probe or a plurality of nucleotide probes which can hybridize with polynucleotide according to any one of claim 1 encoding a polypeptide that mediates phosphate uptake into the plant cell and/or a polynucleotide according to any one of claim 18 representing promoter sequences, or a fragment or a variant thereof, or a complementary sequence thereto and the sample to be assayed.
(b) detecting the hybrid complex formed between the probe and a nucleotide in the sample.

58. A kit for detecting the presence of a polynucleotide according to claim 1 encoding a polypeptide that mediates phosphate uptake into the plant cell and/or a polynucleotide according to claim 18 representing promoter sequences, or a fragment or a variant thereof, or a complementary sequence thereto in a sample, the kit including a nucleotide probe or a plurality of nucleotide probes which can hybridize with a nucleotide sequence comprised within a polynucleotide according to any one of claim 1 encoding a polypeptide that mediates phosphate uptake into the plant cell and/or a polynucleotide according to claim 18 representing promoter sequences, or a fragment or a variant thereof, or a complementary sequence thereto and, optionally, the reagents necessary for performing the hybridization reaction.

59. A method of modifying the frequency of a phophate transporter gene in a plant population, comprising the steps of:
a) screening a plurality of plants using an oligonucleotide as a marker to determine the presence or absence of a phophate transporter gene in an individual plant, the oligonucleotide consisting of not more than 300 bases of a nucleotide sequence according to claim 1,
b) selecting at least one individual plant for breeding based on the presence or absence of the phophate transporter gene; and
c) breeding at least one plant thus selected to produce a population of plants having a modified frequency of the phophate transporter gene.

60. A method according to claim 59, wherein the oligonucleotide comprises a simple sequence repeat (SSR) sequence comprising at least two consecutive repeat units of an SSR, and a flanking sequence of at least about 14 nucleic acids immediately adjacent to said at least two consecutive repeat units.

61. A method of plant breeding to select for or against a trait of interest, which is associated with the efficiacy of phosphate transport in the plant comprising the steps of:
a. identifying the trait of interest; identifying at least one oligonucleotide that can be used as a marker for the trait, the oligonucleotide consisting of not more than 300 bases of a nucleotide sequence according to claim 1,
b. screening at least one plant for the presence of the at least one oligonucleotide;
c. selecting at least one plant based on presence or absence of the at least one oligonucleotide;
d. breeding at least one plant thus selected to produce a population of plants having a modified frequency of the at least one oligonucleotide; and
e. screening at least one plant of the population for the presence or absence of the trait.

62. A method according to claim 61, wherein the oligonucleotide comprises a simple sequence repeat (SSR) sequence comprising at least two consecutive repeat units of an SSR, and a flanking sequence of at least about 14 nucleic acids immediately adjacent to said at least two consecutive repeat units.

63. A method of determining a varietal identity of a plant, comprising:
a. obtaining a nucleic acid sample from a plant;
b. identifying at least one oligonucleotide to obtain an oligonucleotide profile for the plant, wherein the oligonucleotide consists of not more than 300 bases of a nucleotide sequence according to claim 1, the oligonucleotide comprising a simple sequence repeat (SSR) sequence comprising at least two consecutive repeat units of an SSR, and a flanking sequence of at least about 14 nucleic acids immediately adjacent to said at least two consecutive repeat units in the sample; and
c. comparing the SSR profile to at least one known SSR profile corresponding to at least one known variety to determine the varietal identity of the plant.

64. An oligonucleotide primer consisting of between 8 and 150 bases which comprises at least 14 bases selected from the group of flanking sequences obtainable from a nucleotide sequence according to claim 1, which at least 14 bases are immediately adjacent to at least two consecutive repeat units of an SSR.

65. A computer-readable medium having stored thereon a data structure comprising:
a) a nucleic acid molecule that has at least 70% nucleic acid sequence identity to a nucleotide molecule selected from the group consisting of SEQ ID NOs: 1-13, 27, 103 and 105, and the complement thereof; and
b) a module receiving the nucleic acid molecule which compares the nucleic acid sequence of the molecule to at least one other nucleic acid sequence.

66. A computer-readable medium having stored thereon a data structure comprising:
a) nucleic acid molecule that has at least 70% nucleic acid sequence identity to a nucleotide molecule selected from the group consisting of SEQ ID NOs: 29-42, and the complement thereof; and
b) a module receiving the nucleic acid molecule which compares the nucleic acid sequence of the molecule to at least one other nucleic acid sequence.
